(19) United States
(12) Patent Application Publication
(10) Pub. No.: US 2023/0321190 A1
(43) Pub. Date: Oct. 12, 2023

(54) PHARMACEUTICAL AND COSMETIC COMPOSITIONS COMPRISING SECRETOMES

(71) Applicant: ACCELERATED BIOSCIENCES CORP., Carlsbad, CA (US)

(72) Inventors: Jau-Nan LEE, Kaohsiung (TW); Tony Tung-Yin LEE, Yakima, WA (US); Yuta LEE, Kaohsiung (TW)

(21) Appl. No.: 17/969,163

(22) Filed: Oct. 19, 2022

Related U.S. Application Data
(63) Continuation of application No. PCT/US2021/030681, filed on May 4, 2021.
(60) Provisional application No. 63/020,250, filed on May 5, 2020.

(51) Int. Cl. A61K 38/19; A61K 38/20; A61K 38/18; A61K 9/127; A61P 17/00; A61K 45/06; A61K 38/39; A61K 31/201; A61K 31/728

(52) U.S. Cl. CPC A61K 38/195; A61K 9/127; A61K 31/201; A61K 31/728; A61K 38/1866; A61K 38/204; A61K 38/2053; A61K 38/39; A61K 45/06; A61P 17/00

(57) ABSTRACT

Disclosed are pharmaceutical or cosmetic compositions comprising secretomes, for example secreted proteins from stem cells, and uses thereof. A composition that contains a secretome and an acceptable excipient may be free of a cell. The compositions are useful for inducing an immune response, treating an inflammatory response, treating a microbial infection, differentiating cells, wound healing, embryonic development, placental development, central nervous system development, or morphogenesis.

### CROSS-REFERENCE

This application is a continuation of International Patent Application No. PCT/US2021/30681, filed May 4, 2021, which claims the benefit of U.S. Provisional Application No. 63/020,250, filed May 5, 2020, which application is incorporated herein by reference in its entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY OF THE INVENTION

The inventive embodiments provided in this Brief Summary of the Invention are meant to be illustrative only and to provide an overview of selective embodiments disclosed herein. The Brief Summary of the Invention, being illustrative and selective, does not limit the scope of any claim, does not provide the entire scope of inventive embodiments disclosed or contemplated herein, and should not be construed as limiting or constraining the scope of this disclosure or any claimed inventive embodiment.

A secretome disclosed herein can comprise a chemokine, an interleukin, a growth factor, or any combination thereof. A secretome disclosed herein can comprise micro-vesicles, exosomes, or a combination thereof. In some of many aspects, disclosed herein is a composition, comprising 1) about 0.1% or more w/w of secretome and 2) a pharmaceutically or cosmetically acceptable excipient, wherein the secretome comprise monocyte chemoattractant protein-1 (MCP-1; CCL2), and wherein the composition is free from a cell. In some instances, the secretome comprises MCP-1 and one or more of Chemokine (C-X-C motif) ligand 2 (CXCL2; GRO), interleukin 6 (IL-6), IL-8, and vascular endothelial growth factor (VEGF) proteins. In some instances, the secretome comprises MCP-1 and two or more of CXCL2 (GRO), IL-6, IL-8, and VEGF proteins. In some instances, the secretome comprises MCP-1 and three or more of CXCL2 (GRO), IL-6, IL-8, and VEGF proteins. In some instances, the secretome comprises MCP-1 and all of CXCL2 (GRO), IL-6, IL-8, and VEGF proteins.

The secretome can comprise at least: 0.6%, 1%, 1.25%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% of the composition. In some instances, the secretome comprises about 0.6%, about 1%, about 1.25%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of the composition. In some instances, the secretome comprises from about 0.6% to about 25% of the composition, or from about 2.5% to about 10% of the composition. In some instances, the composition is a fluid or gel that comprises from about 100 ng/ml to about 200 ng/ml of MCP-1.

In some aspects, disclosed herein is a composition that comprises MCP-1 and one or more additional proteins of IL-6, VEGF, platelet-derived growth factor AA (PDGF-AA), IL-8, or CXCL2 (GRO); and a pharmaceutically or cosmetically acceptable excipient, wherein a ratio of the MCP-1 and the additional protein is in a range of from about 30: 1 to about 60: 1.

In some aspects, disclosed herein is a composition that comprises a secretome and a pharmaceutically or cosmetically acceptable excipient, wherein the secretome comprises MCP-1 and one or more additional proteins of IL-6, VEGF, PDGF-AA, IL-8, or CXCL2 (GRO). Altematively, disclosed herein is a composition that comprises a secretome and a pharmaceutically or cosmetically acceptable excipient, wherein the secretome comprises MCP-1, CXCL2 (GRO), and one or more additional proteins of IL-8, MCP-3, IL-6, G-CSF, or VEGF., In one instance, a ratio of MCP-1 and IL-8 is in a range from about 10: 1 to about 7:1, the ratio of MCP-1 and MCP-3 is in a range from about 10: 1 to about 30: 1, the ratio of MCP-1 and IL-6 is in a range from about 30: 1 to about 50: 1, the ratio of MCP-1 and G-CSF is in a range from about 30: 1 to about 50: 1, the ratio of MCP-1 and VEGF is in a range from about 30: 1 to about 50: 1, the ratio of CXCL2 and IL-8 is in a range from about 3: 1 to about 4:1, the ratio of CXCL2 and MCP-3 is in a range from about 5: 1 to about 15:1, the ratio of CXCL2 and IL-6 is in a range from about 10: 1 to about 20:1, and/or the ratio of CXCL2 and G-CSF is in a range from about 10: 1 to about 20:1, the ratio of CXCL2 and VEGF is in a range from about 10: 1 to about 20:1, or any combination thereof.

In some aspects, disclosed herein is a composition, wherein the composition comprises a liposome and a pharmaceutically or cosmetically acceptable excipient, wherein the liposome comprises a phospholipid and secretome, and wherein the composition is free from a cell. A secretome can be encapsulated in the liposome. Altematively, the liposome can be in a form of nanoparticles. In some instances, the nanoparticles have an average particle size of from about 10 to about 400 nanometers. In some instances, the nanoparticles have an average particle size of from about 50 to about 300 nanometers. In some instances, the nanoparticles have an average particle size of from about 100 to about 200 nanometers.

In some instances, an exosome carries a chemokine that comprises CXCL2 (GRO), MCP-1, Fractalkine, Interferon gamma-induced protein 10 (IP-10), MCP-3, Eotaxin, Macrophage inflammatory protein-1β (MIP-1β), or any combination thereof. In some instances, the exosome carries an interleukin that comprises IL-6, IL-8, IL-4, IL-1RA, IL-10, IL-12P40, IL-15, IL-1α, IL-17A, or any combination thereof. In some instances, the exosome carries a growth factor that comprises PDGF-AA, VEGF, bFGF, G-CSF, Flt-3L, GM-CSF, or any combination thereof. In some instances, the secretome comprise MCP-1 and one, two, three, or all of CXCL2 (GRO), IL-6, IL-8, and VEGF proteins. In some instances, the secretome comprises MCP-1 and CXCL2 in a weight ratio of from about 1:1 to about 2:1. In some instances, the secretome comprise MCP-1 and CXCL2 in a weight ratio of from about 3:1 to about 4:1. In some instances, the secretome comprise MCP-1 and IL-6 in a weight ratio of from about 2: 1 to about 3:1. In some instances, the secretome comprises MCP-1 and IL-6 in a weight ratio of from about 3: 1 to about 4:1. In some instances, the secretome comprises MCP-1 and IL-8 in a weight ratio of from about 4: 1 to about 6:1. In some instances, the secretome comprises MCP-1 and VEGF in a weight ratio of from about 4: 1 to about 6:1. In some instances, the secretome comprises MCP-1 and VEGF in a weight ratio of from about 7: 1 to about 9:1. In some instances, a secretome further comprises PDGF-AA, and MCP-1 and PDGF-AA are present in the secretome in a weight ratio of from about 3:1 to about 5:1. In some instances, the secretome further comprises PDGF-AA, and MCP-1 and PDGF-AA are present in the secretome in a weight ratio of from about 6:1 to about 9:1. In some instances, the secretome further comprises PDGF-AA, and MCP-1 and PDGF-AA are present in the secretome in a weight ratio of from about 30:1 to about 60:1. In some instances, the ratio of the MCP-1 and any one of the CXCL2, IL-6, IL-8, and VEGF proteins in the secretome is in a range from about 30: 1 to about 60:1. In some instances, the secretome comprise MCP-1, CXCL2, IL-6, IL-8, and VEGF proteins. In some instances, the secretome comprise MCP-1, CXCL2 (GRO), and one, two, three, four or all proteins of IL-8, MCP-3, IL-6, G-CSF, and VEGF. In some instances, the secretome comprises MCP-1 and CXCL2 in a weight ratio of from about 2:1 to about 3:1. In some instances, the secretome further comprises IL-8, and wherein the ratio of MCP-1 and IL-8 is in a range from about 10: 1 to about 6:1 and/or the ratio of CXCL2 and IL-8 is in a range of from about 3: 1 to about 4:1. In some instances, the secretome further comprise MCP-3, and wherein the ratio of MCP-1 and MCP-3 is in a range from about 10: 1 to about 30:1 and/or the ratio of CXCL2 and MCP-3 is in a range from about 5: 1 to about 15:1. In some instances, the secretome further comprise IL-6, and wherein the ratio of MCP-1 and IL-6 is in a range from about 30: 1 to about 50:1 and/or the ratio of CXCL2 and IL-6 is in a range from about 10: 1 to about 20:1. In some instances, the secretome further comprise G-CSF, and wherein the ratio of MCP-1 and G-CSF is in a range from about 30: 1 to about 50:1 and/or the ratio of CXCL2 and G-CSF is in a range from about 10: 1 to about 20:1. In some instances, the secretome further comprise VEGF, the ratio of MCP-1 and VEGF is in a range from about 30: 1 to about 50: 1, and the ratio of CXCL2 and VEGF is in a range from about 10: 1 to about 20:1. In some instances, the composition further comprises one or more proteins of IP-10, Eotaxin, Flt-3L, GM-CSF, MIP-1a, MIP-1b, IL-1a, IL-1RA, IL-4, IL-7, IL-10, IL-12P40, IL-13, IL-15, IL-17A, CCL5 (RANTES), MDC, MCP-3, IL-12P70, IFN-alpha, IFNR, PDGF-AB/BB, or EGF.

Altematively, or in addition, the composition disclosed herein comprises a hydrophilic active agent. Altematively, or in addition, the composition comprises a vitamin. Altematively, or in addition, the composition comprises a hydrophobic active agent. Altematively, or in addition, the composition comprises a fatty acid molecule. Altematively, or in addition, the composition comprises linoleic acid. Alternatively, or in addition, the composition comprises collagen. Altematively, or in addition, the composition comprises hyaluronic acid. Alternatively, or in addition, the composition is free from a serum, antibiotic, or a combination thereof. Altematively, or in addition, the composition is free from steroid, cholesterol, choline chloride, hypoxanthine-sodium salt, thymidine, putrescine dihydrochloride, ferric nitrate, L-glutamine, or any combination thereof. Altematively, or in addition, the composition is free from a color additive.

A composition described herein can be suitable for administration as a cosmeceutical composition or a pharmaceutical composition. In some instances, the composition is a dosage form of a lotion, cream, liquid, gel, emulsion, suspension, paste, stick, aerosol, foam, patch, powder, ointment, bead, mask, pad, sheet, wound dressing, bandage, or any combination thereof. In some instances, the pharmaceutically or cosmetically acceptable excipient comprises sterile water, phosphate buffered saline, a surfactant, glycerol, a seed oil, a fruit oil, a flower extract, a mineral oil, a synthetic oil, a saccharide, a silicate, a calcium salt, a magnesium salt, sodium chloride, sodium hydroxide, potassium chloride, lactose, lactic acid, a starch, a sugar alcohol, a cellulose, an activated charcoal, an amino acid, a paraffin, honey, a wax, beeswax, an agar, calcium carbonate, a citric acid, tartaric acid, a steric acid, xanthan gum, benzoic acid or salt thereof, a polyethylene glycol, a silicon, or any combination thereof.

In some aspects, disclosed herein is a method, comprising contacting a composition disclosed herein with a subject in need thereof. In some instances, the method treats a disease in the subject. In some instances, the method ameliorates a condition of the subject’ skin. In some instances, the disease or condition is eczema, rash, psoriasis, acne, rosacea, ichthyosis, vitiligo, hive, seborrheic dermatitis, shingles, burn, sunburn, contact dermatitis, wrinkled skin, scarred skin, sagging skin, loss of skin elasticity, skin dryness, skin dullness, or any combination thereof.

In one aspect, provided herein is a method of producing one or more proteins of interest from a trophoblastic cell line, the method comprising: culturing human trophoblastic stem cells with a nutritional media until confluency is reached; inducing hypoxia; and isolating the one or more proteins of interest from the media. In some instances, hypoxia is induced for approximately 12-48 hours and, in some instances, is induced for about 24 hours. Confluency can vary depending upon the culture dish being utilized. Non-limiting examples of confluency can comprise from about 3,000 cells/cm² to about 9,000 cells/cm², from about 4,000 cells/cm² to about 8,000 cells/cm², from about 5,000 cells/cm² to about 7,000 cells/cm², or about 6,000 cells/cm². In some instances, the isolated one or more proteins can, optionally, be further mixed with one or more pharmaceutically acceptable excipients in order to prepare a composition.

The described method can produce one or more proteins that can comprise a cytokine, a growth factor, a membrane-bound signaling molecule, a cell adhesion molecule, a defense protein, an immunity protein, and extracellular matrix protein, an intracellular signal molecule, a metabolite interconversion enzyme, a protein modifying enzyme /protease, a protein-binding modulator / protease inhibitor, a scaffold/adaptor protein, a structural protein, a transfer protein or a carrier protein, a transmembrane signal receptor, or any combination thereof. A secretome produced from any one of the described methods is useful in one or more of the following processes: biological adhesion/cell adhesion, biological regulation, cell proliferation, cellular component organization or biogenesis, a cellular process (e.g., cell activation, cell communication, cell cycle process, cell death, cell growth, cellular component organization, cellular developmental process, cell differentiation, cellular component morphogenesis, cellular metabolic process, cellular response to stimulus, export from cell, microtubule-based process, cell activation, cell communication, cell cycle process, cell death, cell growth, cellular component organization, cellular developmental process (e.g., cell differentiation, or cellular component morphogenesis), cellular metabolic process, cellular response to stimulus, export from cell, microtubule-based process, movement of cell or subcellular component, cell motility, neuron projection guidance, myelination, signal transduction, etc.), developmental process (e.g., anatomical structure development, anatomical structure formation involved in morphogenesis, anatomical structure morphogenesis, cellular developmental process, developmental growth, etc.), growth, an immune system process (e.g., immune effector process, immune response, immune system development, leukocyte activation, leukocyte migration, etc.), localization (e.g., cellular localization, establishment of localization, localization of cell, macromolecule localization, etc.), a locomotion (e.g., cell motility, taxis, etc.) a metabolic processes (e.g., biosynthetic process, catabolic process, cellular metabolic process, hormone metabolic process, nitrogen compound metabolic process, etc.), a multi-organism process / response to other organism, a multicellular organism process (e.g., coagulation, cytokine production, digestion, multicellular organism development, system process, etc.), a response to stimulus (e.g., cellular response to stimulus, immune response, etc.), signaling (e.g., Cell-cell signaling, Signal transduction, etc.).

A secretome produced from any one of the described methods is useful in one or more of the following pathways: Alzheimer disease-amyloid secretase pathway; Alzheimer disease-presenilin pathway; angiogenesis; apoptosis signaling pathway; axon guidance mediated by Slit/Robo; Axon guidance mediated by netrin; blood coagulation; CCKR signaling map; cadherin signaling pathway; endothelin signaling pathway; FAS signaling pathway; gonadotropin-releasing hormone receptor pathway; inflammation mediated by chemokine and cytokine signaling pathway; insulin/IGF pathway — MAPKK/MAPK cascade; insulin/IGF pathway— PKB signaling cascade; interleukin signaling pathway; PDGF signaling pathway; plasminogen activating cascade; T cell activation; TGF-beta signaling pathway; toll receptor signaling pathway; Wnt signaling pathway; P53 pathway, etc.

Provided herein is a method, comprising administering to a subject in need thereof any of any of such compositions. Provided herein is a use of any of any of such compositions for treating a subject in need thereof, or for the manufacture of a medicament for treating a subject in need thereof. Provided herein is a use of any of any of such compositions, for use in an in vitro culture or assay.

In any of such compositions, the composition can be substantially free from a cell. Altematively, the composition can be free of cells.

The secretome can be present in the composition in an amount of from about 0.1% to about 75% by weight, from about 0.1% to about 65% by weight, from about 0.1% to about 50% by weight, from about 0.1% to about 40% by weight, from about 0.1% to about 30% by weight, from about 0.1% to about 20% by weight, from about 0.1% to about 15% by weight, from about 0.1% to about 10% by weight, or from about 0.1% to about 5% by weight.

In any of such embodiments, the secretome can comprise at least: 0.6%, 1%, 1.25%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% of the composition. In some instances, the secretome comprises about 0.6%, about 1%, about 1.25%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of the composition. In some instances, the secretome comprises from about 0.6% to about 25% of the composition, or from about 2.5% to about 10% of the composition.

When a secretome contains more than one protein, each protein can be present in a ratio of from about 1:1 to about 20:1. For example, each protein can be present in a ratio of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1.

In some cases, a composition disclosed herein can be aseptic. In some cases, the composition can comprise one or more resident microbes or cells. The one or more microbes or cells can be viruses, bacteria, eukaryotic cells, or any combination thereof. In some instances, the one or more microbes or cells may not be pathogenic. In some instances, the composition can comprise a bacterium or bacteria at a concentration of less than 10 colony forming units (CFU)/gram (g), 50 CFU/g, 100 CFU/g, 150 CFU/g, 200 CFU/g, 300 CFU/g, 400 CFU/g, 500 CFU/g, 600 CFU/g, 700 CFU/g, 800 CFU/g, 900 CFU/g, or 1000 CFU/g. In some cases, the composition can comprise bacteria at a concentration of from about 10 CFU/g to about 1000 CFU/g, from about 10 CFU/g to about 50 CFU/g, from about 20 CFU/g to about 100 CFU/g, from about 50 CFU/g to about 200 CFU/g, from about 100 CFU/g to about 250 CFU/g, from about 200 CFU/g to about 500 CFU/g, from about 500 CFU/g to about 700CFU/g, or from about 600 CFU/g to about 1000 CFU/g. In some instances, the composition may be substantially free of, or free of, Staphylococcus aureus, Streptococcus pyogenes, Pseudomonas aeruginosa, Pseudomonas species, Klebsiella pneumoniae, or any combination thereof.

In some cases, a composition disclosed herein may not contain a heavy metal such as, for example, lead, bithionol, chlorofluorocarbon propellants, nitrosamines, chloroform, halogenated salicylanilides, hexachlorophene, mercury compounds, 1,4-dioxane, methylene chloride, prohibited cattle materials, sunscreen compounds, vinyl chloride, zirconium-containing complexes, or any combination thereof. In some instances, the prohibited cattle materials can comprise the brain, skull, eyes, trigeminal ganglia, spinal cord, vertebral column, dorsal root ganglia, tonsils, distal ileum of the small intestine or any combination thereof. In some instances, the composition may comprise lead at levels of 10 (parts per million) ppm or less.

In some cases, a composition herein does not comprise a color additive. In some cases, the composition can comprise a color additive. In some cases, the composition may contain an incidental ingredient such as a color additive in an insignificant level in the composition, for example less than: 5%, 4%, 3%, 2%, 1%, 0.5%, or 0.1%. In some cases, the incidental ingredient may have no technical/structural, functional or any combination thereof effect in the composition, e.g., an incidental ingredient is not an active ingredient.

In some instances, the composition can comprise a protein at a concentration of less than 1 nanogram/milliliter (ng/ml), 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 30 ng/ml, 35 ng/ml, 40 ng/ml, 45 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, or 10000 ng/ml. In some instances, the composition can comprise a protein at a concentration of: from about 1 ng/ml to about 100 ng/ml, from about 10 ng/ml to about 200 ng/ml, from about 10 ng/ml to about 400 ng/ml, from about 50 ng/ml to 300 ng/ml, from about 100 ng/ml to about 200 ng/ml, from about 150 ng/ml to about 400 ng/ml, from about 200 ng/ml to about 600 ng/ml, from about 400 ng/ml to about 700 ng/ml, from about 500 ng/ml to about 900 ng/ml, from about 600 ng/ml to about 1000 ng/ml, from about 900 ng/ml to about 1500 ng/ml, or from about 1000 ng/ml to about 10000 ng/ml.

In some instances, a secretome can comprise at least 0.01%, 0.1%. 1%, 1.25%, 2%, 3%, 4%, 5%, 6%, 7%, 8%, 9%, 10%, 20%, 30%, 40%, 50%, 60%, 70%, 80%, 90%, 95%, or 99% of the composition. In some instances, the secretome can comprise from about: 0.01% to about 0.1% of the composition, from about 0.01% to about 1% of the composition, from about 1% to about 2% of the composition, from about 1% to about 5% of the composition, from about 3% to about 8% of the composition, from about 5% to about 10% of the composition, from about 10% to about 20% of the composition, from about 20% to about 40% of the composition, from about 30% to about 50% of the composition, from about 50% to about 75% of the composition, from about 60% to about 90% of the composition, from about 75% to about 95% of the composition, or from about 80% to about 99% of the composition.

In some cases, a composition described herein can comprise an exosome, a liposome, a nanoparticle, or any combination thereof. In some instances, a liposome can be in a form of a nanoparticle. In some instances, a nanoparticle can comprise a liposome. In some cases, the exosome, the liposome, the nanoparticle, or any combination thereof, can comprise the secretome, a phospholipid, a protein, a hydrophilic active agent, a hydrophilic active agent, a vitamin, an inactive ingredient, or any combination thereof. The liposome can include, but may not be limited to, a unilamellar liposome, a multilamellar liposome, an archaeosome, a noisome, a novasome, a cryptosome, an emulsome, a vesosome, a nanoliposome, a nanoemulsion, or a derivative of any of these, or any combination thereof. The nanoparticle can include, but may not be limited to, a biopolymeric nanoparticle, an alginate nanoparticle, a xanthan gum nanoparticle, a cellulose nanoparticle, a lipid nanoparticle, a dendrimer, a polymeric micelle, a polyplexed, an inorganic nanoparticle, a nanocrystal, a metallic nanoparticle, a quantum dot, a protein nanoparticle, a polysaccharide nanoparticle, a derivative of any of these, or any combination thereof.

In some instances, a nanoparticle can be less than 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm. In some instances, the nanoparticle can be more than 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm. In some instances, the nanoparticles can have an average particle size of from about 1 nm to about 100 nm, from about 10 nm to about 200 nm, from about 10 nm to about 400 nm, from about 50 nm to 300 nm, from about 100 nm to about 200 nm, from about 150 nm to about 400 nm, from about 200 nm to about 600 nm, from about 400 nm to about 700 nm, from about 500 nm to about 900 nm, from about 600 nm to about 1000 nm, or from about 700 nm to about 1500 nm.

In any of such aspects, embodiments, and/or instances, the inventors have demonstrated stem cells are immune-privileged, chromosomally stable (not tumorigenic), pathogen free, and pluripotent. The inventors have also demonstrated efficient differentiation of its stem cells with remarkable doubling times and growth characteristics to programmed natural killer (NK), cartilage, bone, fat, neuron, pancreas, liver, and secretome cells.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D show secretome composition profiles. FIG. 1A represents hTSC secretome secretion based on the average of 2 cell lines minus the negative control, which is medium only. The graph illustrates the highest concentration of proteins and is organized in descending order.

FIG. 1B represents hTSC-derived prCTBs and pancreatic progenitor cells (24h with bFGF). The prCTB/PPC secretion based on the average of 2, cell lines (1808 and 1808-3E2 monoclone) minus the negative control, which is medium only. The graph illustrates the highest concentration of proteins and is organized in descending order. FIG. 1C represents hTSC-derived neural progenitor cells (24h with RA). The NSC secretion is based on the average of 2 cell lines (1808 and 1808-3E2 monoclone) minus the negative control, which is medium only. The graph illustrates the highest concentration of proteins and is organized in descending order.

FIG. 1D represents hTSC-derived hepatocyte-like cells (8h with bFGF + 5-7 d with Dexa, OSM, BMP4, HGF). The HPC secretion is based on the average of 2 cell lines (1808 and 1808-3E2 monoclone) minus the negative control, which is medium only. The graph illustrates the highest concentration of proteins and is organized in descending order.

FIGS. 2A-2C show additional secretome composition profiles. FIG. 2A represents hTSC-derived neural progenitor cells (24 h with RA). The NSC secretion data is the same as from FIG. 1C and is in more detail and sub-grouped by Chemokines, Cytokines, and Growth Factors. FIG. 2B represents hTSC-derived prCTBs and pancreatic progenitor cells (24 h with bFGF). The prCTB/PPC secretion data is the same as from FIG. 1B and is in more detail and sub-grouped by Chemokines, Cytokines, and Growth Factors. FIG. 2C represents hTSC-derived hepatocyte-like cells (8 h with bFGF + 5-7 d with Dexa, OSM, BMP4, HGF). The HPC secretion data is the same as from FIG. 1D and is in more detail and sub-grouped by Chemokines, Cytokines, and Growth Factors.

FIG. 3 shows results of an MTT assay of skin cell viability in the presence of an exemplary secretome formulation herein. Cell viability is illustrated at 48 (solid bar), 72 (hatch bar), or 96 hours (diagonal bar) at various concentrations of the secretome formulation compared to control. * = statistical significance compared to control.

FIGS. 4A and 4B show results of a skin cell transwell migration assay in the presence of MCP-1 compared to control at 4, 6, and 8 hours of culture, presented in a bar chart (FIG. 4A) and electron microscopy images of control versus MCP-1 at 4, 6, and 8 hours of culture. (FIG. 4B).

### DETAILED DESCRIPTION

The details of one or more inventive embodiments are set forth in the accompanying drawings, the claims, and the description herein. The features, compound, compositions, methods, and advantages of the inventive disclosure herein can be combined with any other feature, compound, composition, method, advantages disclosed herein unless explicitly excluded.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range, e.g., ± 15% of a referenced numeral value. About also includes the exact amount, for example “about 5 µL” means “about 5 µL” and also “5 µL.” Generally, the term “about” includes an amount that would be expected to be within experimental error.

The terms “treating,” “treatment,” and the like are used herein to mean obtaining a desired pharmacologic and/or physiologic effect. In some instances, an individual is treated therapeutically (e.g., when an individual is suffering from a liver-associated disease or disorder), such therapeutic treatment causes a partial or complete cure or treatment for the disease or disorder, and/or reverses an adverse effect attributable to the disease or disorder, and/or stabilizes the disease or disorder, and/or delays progression of the disease or disorder, and/or causes regression of the disease or disorder. In some instances, a subject is prophylactically treated (e.g., an individual suspected to be suffering from and/or genetically pre-disposed to a liver-associated disease or disorder is treated prophylactically with a preparation of cells described herein and such prophylactic treatment completely or partially prevents a liver-associated disease or disorder or sign or symptom thereof.

Administration disclosed herein to an area in need of treatment is achieved by, for example and not by way of limitation, local infusion (e.g., during surgery), by injection, by means of a catheter, or by means of an implant. An implant can be of a porous, non-porous, or gelatinous material including, but not limited to, membranes such as silastic membranes or fibers.

An “effective amount” is an amount of a therapeutic agent sufficient to achieve the intended purpose. An effective amount of a composition to treat or ameliorate a disease or disorder is an amount of the composition sufficient to reduce or remove the symptoms of the disease or disorder.

The section headings used herein are for organizational purposes only and are not to be construed as limiting the subject matter described.

### Compositions

In some aspects, disclosed herein is a composition that comprises a secretome, e.g., a protein, an exosome, a microvesicle, secreted from a cell or a population of cells (e.g., example stem cells). In some instances, the stem cells can be pluripotent. In some instances, the stem cells can be mortal. In some instances, the stem cells may not be embryonic stem cells. In some instances, the stem cells may be derived from trophoblast tissue. In some instances, the stem cells may be mortal pluripotent stem cells or cells differentiated therefrom. In some instances, the secretome is isolated or purified and is not present in a host organism or stem cell, from which the secretome may be derived. In some instances, the secretome is purified or extracted from a stem cell culture or medium. In some instances, the secretome may be one or more proteins comprising a cytokine, a chemokine, a growth factor, a soluble molecule, or any combination thereof. In some instances, the one or more proteins can be separate from exosomes or microparticles. In some instances, the one or more proteins can be on the surface of exosomes or microparticles. In some instances, the one or more proteins can be encapsulated by exosomes or microparticles. In some instances, the exosomes have an average particle diameter of about 500 nm or less, e.g., about 250 nm or less or, for example, from about 50 to about 150 nm. In some cases, the composition comprises one or more pharmaceutically and/or cosmetically acceptable excipients.

Disclosed herein is a composition that comprises 1) about 0.1% or more w/w of secretome and 2) a pharmaceutically or cosmetically acceptable excipient, wherein the secretome comprises MCP-1, and wherein the composition is free from a cell. Disclosed herein is a cosmeceutical composition that comprises 1) about 0.1% or more w/w of secretome and 2) one or more cosmetically acceptable excipients, wherein the secretome comprises MCP-1, and wherein the composition is free from a cell. Disclosed herein is a pharmaceutical composition that comprises 1) about 0.1% or more w/w of secretome and 2) one or more pharmaceutically acceptable excipients, wherein the secretome comprises MCP-1, and wherein the composition is free from a cell. In some instances, the secretome comprises MCP-1 and one of CXCL2 (GRO), IL-6, IL-8, and VEGF proteins. In some instances, the secretome comprises MCP-1 and two of CXCL2 (GRO), IL-6, IL-8, and VEGF proteins. In some instances, the secretome comprises MCP-1 and three of CXCL2 (GRO), IL-6, IL-8, and VEGF proteins. In some instances, the secretome comprises MCP-1 and all of CXCL2 (GRO), IL-6, IL-8, and VEGF proteins. In some instances, the secretome comprises at least: 0.6%, 1%, 1.25%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% of the composition. In some instances, the secretome comprises about 0.6%, about 1%, about 1.25%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of the composition. In some instances, the secretome comprises from about 0.6% to about 25% of the composition. In some instances, the secretome comprises from about 2.5% to about 10% of the composition. In some instances, the composition is a fluid or gel and comprises from about 100 ng/ml to about 200 ng/ml of a secretome protein such as, for example, MCP-1. In some instances, a secretome protein can be present in the composition in an amount of about 0.1-1 ng/ml, about 1-100 ng/ml, about 20-100 ng/ml, about 200-500 ng/ml, or about 500-1000 ng/ml. Where more than one protein is present in a composition, each protein can be present in the composition in an amount of about 0.1-1 ng/ml, 1-100 ng/ml, 20-100 ng/ml, 200-500 ng/ml, or 500-1000 ng/ml in the composition. Altematively, where more than one protein is present in a composition, all of the proteins in combination can be present in the composition in an amount of about 0.1-1 ng/ml, 1-100 ng/ml, 20-100 ng/ml, 200-500 ng/ml, or 500-1000 ng/ml in the composition.

In some aspects, disclosed herein is a composition that comprises MCP-1 and one or more additional proteins of IL-6, VEGF, PDGF-AA, IL-8, or CXCL2 (GRO); and a pharmaceutically or cosmetically acceptable excipient, wherein a weight ratio of MCP-1 and each of the one or more additional proteins is in a range from about 30: 1 to about 60:1, for example from about 30:1 to about 50:1, from about 30:1 to about 40:1, or from about 40:1 to about 50:1. In some instances, MCP-1 is present in the composition in the amount of about 1-20 ng/ml, for example, 1-10 ng/ml, e.g., about 6-7 ng/ml in the composition.

Disclosed herein is a composition that comprises MCP-1 and one or more additional proteins of IL-6, VEGF, PDGF-AA, IL-8, or CXCL2 (GRO); and a pharmaceutically or cosmetically acceptable excipient composition. Altematively, disclosed herein is a composition that comprises MCP-1, and CXCL2 (GRO), and one or more additional proteins of IL-8, MCP-3, IL-6, G-CSF, or VEGF; and a pharmaceutically or cosmetically acceptable excipient. In any of such compositions, a ratio of MCP-1 and IL-8 in the secretome or in the composition is in a range from about 10: 1 to about 7:1, a ratio of MCP-1 and MCP-3 in the secretome or in the composition is in a range from about 10: 1 to about 30:1, a ratio of MCP-1 and IL-6 in the secretome or in the composition is in a range from about 30: 1 to about 50:1, a ratio of MCP-1 and G-CSF in the secretome or in the composition is in a range from about 30: 1 to about 50:1, a ratio of MCP-1 and VEGF in the secretome or in the composition is in a range from about 30: 1 to about 50:1, a ratio of CXCL2 and IL-8 in the secretome or in the composition is in a range from about 3: 1 to about 4:1, a ratio of CXCL2 and MCP-3 in the secretome or in the composition is in a range from about 5: 1 to about 15:1, a ratio of CXCL2 and IL-6 in the secretome or in the composition is in a range from about 10: 1 to about 20:1, a ratio of CXCL2 and G-CSF in the secretome or in the composition is in a range from about 10: 1 to about 20:1, a ratio of CXCL2 and VEGF in the secretome or in the composition is in a range from about 10: 1 to about 20:1, or any combination thereof. In some instances, MCP-1 is present in the secretome or in the composition in an amount of about 1-20 ng/ml, f about 1-10 ng/ml, or about 6-7 ng/ml.

In some instances, a composition herein comprising secretome protein such as, for example, MCP-1, CXCL2, and IL-8 can facilitate an immune response including, but not limited to wound healing and/or angiogenesis.

In some instances, a composition herein comprising secretome protein such as, for example, IL-6, can stimulate energy mobilization that leads to increased circulation in muscle and/or fatty tissue.

In some instances, a composition herein comprising secretome protein such as, for example, PDGF, can regulate cell growth and division in blood vessel, the growth of blood vessels from already-existing blood vessel tissue, mitogenesis, e.g., proliferation, of mesenchymal cells such as fibroblasts, osteoblasts, tenocytes, vascular smooth muscle cells and mesenchymal stem cells as well as chemotaxis, the directed migration, of mesenchymal cells.

In some instances, a composition herein comprising a secretome protein such as, for example, VEGF, can induce blood vessel formation, facilitating in vasculogenesis (the de novo formation of the embryonic circulatory system) and angiogenesis (the growth of blood vessels from pre-existing vasculature) and after injury, or restoring the oxygen supply to tissues when blood circulation is inadequate such as in hypoxic conditions.

In some instances, a composition herein can facilitate one or more stages of wound healing including, but not limited to, inflammation (via, for example, ROS mediation), granulation ECM formation, wound closure, and/or remodeling ECM reorganization strengthening.

In some aspects, disclosed herein is a composition, that comprises a liposome and a pharmaceutically or a cosmetically acceptable excipient, wherein the liposome comprises a phospholipid and secretome, and the composition is free from cells or substantially free from a cell. In some instances, the secretome is encapsulated in the liposome. In some instances, the liposome is in a form of nanoparticles. In some instances, the nanoparticles have an average particle size of from about 10 to about 400 nanometers. In some instances, the nanoparticles have an average particle size of from about 50 to about 300 nanometers. In some instances, the nanoparticles have an average particle size of from about 100 to about 200 nanometers.

In some instances, a secretome comprises a chemokine, an interleukin, a growth factor, or any combination thereof. In some instances, a secretome comprise micro-vesicles, exosomes, or a combination thereof. In some instances, an exosome comprises a chemokine that comprises CXCL2, MCP-1, Fractalkine, IP-10, MCP-3, Eotaxin, MIP-1β, or any combination thereof. In some instances, an exosome comprises an interleukin that comprises IL-6, IL-8, IL-4, IL-1RA, IL-10, IL-12P40, IL-15, IL-1α, IL-17A, or any combination thereof. In some instances, an exosome comprises a growth factor that comprises PDGF-AA, VEGF, bFGF (FGF-2), G-CSF, Flt-3L, GM-CSF, or any combination thereof. In some instances, a secretome comprises MCP-1 and one, two, three, or all of CXCL2 (GRO), IL-6, IL-8, and VEGF proteins. In some instances, a secretome comprises MCP-1 and CXCL2 in a weight ratio of from about 1:1 to about 2:1. Altematively, or in addition, a secretome comprises MCP-1 and CXCL2 in a weight ratio of from about 3:1 to about 4:1. Altematively, or in addition, a secretome comprises MCP-1 and IL-6 in a weight ratio of from about 2: 1 to about 3:1. Altematively, or in addition, a secretome comprises MCP-1 and IL-6 in a weight ratio of from about 3: 1 to about 4:1. Altematively, or in addition, a secretome comprises MCP-1 and IL-8 in a weight ratio of from about 4: 1 to about 6:1. Alternatively, or in addition, a secretome comprises MCP-1 and VEGF in a weight ratio of from about 4: 1 to about 6:1. Alternatively, or in addition, a secretome comprises MCP-1 and VEGF in a weight ratio of from about 7: 1 to about 9:1. Altematively, or in addition, a secretome comprises MCP-1 and PDGF-AA, wherein MCP-1 and PDGF-AA are present in a weight ratio of from about 3:1 to about 5:1. Altematively, or in addition, a secretome comprises MCP-1 and PDGF-AA, wherein MCP-1 and PDGF-AA are present in a weight ratio of from about 6:1 to about 9:1. Altematively, or in addition, a secretome comprises MCP-1 and PDGF-AA, wherein MCP-1 and PDGF-AA are present in a weight ratio of from about 30:1 to about 60:1. In some instances, the ratio of the MCP-1 and any one of the CXCL2, IL-6, IL-8, and VEGF proteins is in a range from about 30: 1 to about 60:1. Altematively, or in addition, a secretome comprises MCP-1, CXCL2, IL-6, IL-8, and VEGF proteins. In some instances, the secretome comprise MCP-1, CXCL2 (GRO), and one, two, three, four, or all proteins of IL-8, MCP-3, IL-6, G-CSF, and VEGF.

Provided herein is a secretome that comprises MCP-1 and CXCL2 in a weight ratio of from about 2:1 to about 3:1. In some instances, the secretome further comprises IL-8, and a weight ratio of MCP-1 and IL-8 in the secretome or in the composition is in a range of from about 10: 1 to about 6:1 and/or a weight ratio of CXCL2 and IL-8 in the secretome or in the composition is in a range of from about 3: 1 to about 4:1. In some instances, the secretome further comprises MCP-3and/or CXCL2, and a weight ratio of MCP-1 and MCP-3 in the secretome or in the composition is in a range from about 10: 1 to about 30:1 and/or a weight ratio of CXCL2 and MCP-3 in the secretome or in the composition is in a range from about 5: 1 to about 15:1. In some instances, the secretome further comprises IL-6 and/or CXCL2, and a weight ratio of MCP-1 and IL-6 in the secretome or in the composition is in a range from about 30: 1 to about 50:1 and/or a weight ratio of CXCL2 and IL-6 in the secretome or in the composition is in a range from about 10: 1 to about 20:1. In some instances, the secretome furthers comprise G-CSF and CXCL2, and a weight ratio of MCP-1 and G-CSF in the secretome or in the composition is in a range from about 30: 1 to about 50:1 and/or a weight ratio of CXCL2 and G-CSF in the secretome or in the composition is in a range from about 10: 1 to about 20:1. In some instances, the secretome further comprise CXCL2 and VEGF, and a weight ratio of MCP-1 and VEGF in the secretome or in the composition is in a range from about 30: 1 to about 50:1, and a weight ratio of CXCL2 and VEGF in the secretome or in the composition is in a range from about 10: 1 to about 20:1. In some instances, the composition further comprises one or more proteins of IP-10, Eotaxin, Flt-3L, GM-CSF, MIP-1a, MIP-1b, IL-1a, IL-1RA, IL-4, IL-7, IL-10, IL-12P40, IL-13, IL-15, IL-17A, CCL5 (RANTES), MDC, MCP-3, IL-12P70, IFN alpha (IFN-α), interferon receptor (IFNR), PDGF-AB/BB, or EGF.

In some instances, the composition comprises two or more proteins of CXCL2 (GRO) CCL5 (RANTES), MCP-1, MCP-3, MDC, Fractalkine, IL-6, IL-8, PGDF-AA, PDGF-AB/BB, VEGF, EGF, and G-CSF. In some instances, the composition comprises CXCL2 (GRO) and CCL5 (RANTES) in a weight ratio of from about 1:1 to about 1:2, or from about 1:1 to about 1:4. In some instances, the composition comprises CXCL2 and MCP-1 in a weight ratio of from about 3:1 to about 1:3, from about 3:1 to about 1:2, or from about 1:1 to about 1:3. In some instances, the composition comprises CXCL2 and PDGF-AA in a weight ratio of from about 2:1 to about 5:1. In some instances, the composition comprises CXCL2 and PDGF-AB/BB in a weight ratio of from about 3:1 to about 4:1, or from about 40:1 to about 60:1. In some instances, the composition comprises CXCL2 and IL-6 in a weight ratio of from about 8:1 to about 10:1, or from about 20:1 to about 30:1. In some instances, the composition comprises CXCL2 and IL-8 in a weight ratio of from about 10:1 to about 15:1, or from about 4:1 to about 5:1. In some instances, the composition comprises CXCL2 and MDC in a weight ratio of from about 15:1 to about 50:1. In some instances, the composition comprises CXCL2 and Fractalkine in a weight ratio of from about 20:1 to about 50:1. In some instances, the composition comprises CXCL2 and MCP-3 in a weight ratio of from about 10:1 to about 30:1, or from about 3:1 to about 5:1. In some instances, the composition comprises CXCL2 and VEGF in a weight ratio of from about 10:1 to about 20:1, from about 10:1 to about 40:1, or from about 20:1 to about 30:1. In some instances, the composition comprises CXCL2 and EGF in a weight ratio of from about 30:1 to about 60:1. In some instances, the composition comprises CXCL2 and G-CSF in a weight ratio of from about 10:1 to about 40:1, or from about 20:1 to about 30:1. In some instances, the composition further comprises IL-10, MCP-3, Exotaxin, MIP-1a, MIP-1b, IL-4, IL-1RA, IL-10, IL-12P40, IL-15, IL-1a, IL-17A, FGF-2 (bFGF), Flt-3L, G-CSF, GM-CSF.

In some instances, the composition disclosed herein comprises a hydrophilic active agent. In some instances, the composition comprises a vitamin. In some instances, the composition comprises a hydrophobic active agent. In some instances, the composition comprises a fatty acid molecule. In some instances, the composition comprises linoleic acid. In some instances, the composition comprises collagen. In some instances, the composition comprises hyaluronic acid.

In some instances, the composition is free from a serum, antibiotic, or a combination thereof. In some instances, the composition is free from steroid, cholesterol, choline chloride, hypoxanthine-sodium salt, thymidine, putrescine dihydrochloride, ferric nitrate, L-glutamine, or any combination thereof. In some instances, the composition is free from a color additive.

In some cases, a composition disclosed herein can be aseptic. In some cases, the composition can comprise one or more resident microbes or cells. The one or more microbes or cells can be viruses, bacteria, eukaryotic cells, or any combination thereof. In some instances, the one or more microbes or cells may not be pathogenic. In some instances, the composition can comprise a bacterium or bacteria at a concentration of less than 10 colony forming units (CFU)/gram (g), 50 CFU/g, 100 CFU/g, 150 CFU/g, 200 CFU/g, 300 CFU/g, 400 CFU/g, 500 CFU/g, 600 CFU/g, 700 CFU/g, 800 CFU/g, 900 CFU/g, or 1000 CFU/g. In some cases, the composition can comprise bacteria at a concentration of from about 10 CFU/g to about 1000 CFU/g, from about 10 CFU/g to about 50 CFU/g, from about 20 CFU/g to about 100 CFU/g, from about 50 CFU/g to about 200 CFU/g, from about 100 CFU/g to about 250 CFU/g, from about 200 CFU/g to about 500 CFU/g, from about 500 CFU/g to about 700CFU/g, or from about 600 CFU/g to about 1000 CFU/g. In some instances, the composition may be substantially free of, or free of, Staphylococcus aureus, Streptococcus pyogenes, Pseudomonas aeruginosa, Pseudomonas species, Klebsiella pneumoniae, or any combination thereof.

In some cases, a composition disclosed herein may not contain a heavy metal such as, for example, lead, bithionol, chlorofluorocarbon propellants, nitrosamines, chloroform, halogenated salicylanilides, hexachlorophene, mercury compounds, 1,4-dioxane, methylene chloride, prohibited cattle materials, sunscreen compounds, vinyl chloride, zirconium-containing complexes, or any combination thereof. In some instances, the prohibited cattle materials can comprise the brain, skull, eyes, trigeminal ganglia, spinal cord, vertebral column, dorsal root ganglia, tonsils, distal ileum of the small intestine or any combination thereof. In some instances, the composition may comprise lead at levels of 10 (parts per million) ppm or less.

In some cases, a composition herein does not comprise a color additive. In some cases, the composition can comprise a color additive. In some cases, the composition may contain an incidental ingredient such as a color additive in an insignificant level in the composition, for example less than: 5%, 4%, 3%, 2%, 1%, 0.5%, or 0.1%. In some cases, the incidental ingredient may have no technical/structural, functional or any combination thereof effect in the composition, e.g., an incidental ingredient is not an active ingredient.

In some cases, a composition disclosed herein can include an alpha hydroxy acid, a beta hydroxyl acid, diethanolamine (DEA), a talc or any combination thereof. In some instances, a beta hydroxyl acid can be salicylic acid, beta hydroxybutanoic acid, tropic acid, trethocanic acid, a salt thereof or any combination thereof. In some instances, the diethanolamine can comprise cocamide DEA, cocamide monoethanolamine (MEA), DEA-cetyl phosphate, DEA Oleth-3 phosphate, lauramide DEA, linoleamide MEA, myristamide DEA, oleamide DEA, stearamide MEA, TEA-lauryl sulfate, triethanolamine, a salt thereof, or any combination thereof. In some instances, the composition comprises a fragrance, a paraben, a phthalate, an alcohol, or any combination thereof, for example less than: 5%, 4%, 3%, 2%, 1%, 0.5%, or 0.1%. In some instances, the fragrance can be a perfume, cologne, an aftershave, an essential oil, or any combination thereof. In some instances, the composition is free from a fragrance, a paraben, a phthalate, an alcohol, or any combination thereof.

In some cases, an excipient disclosed herein can comprise water, glycerol, saline, a vegetable oil (e.g., seed oil), a fruit oil, a flower extract, a mineral oil, a synthetic oil, a sugar compound, a silicate, a calcium salt, a magnesium salt, sodium chloride, potassium chloride, lactic acid, a starch, a sugar alcohol, a cellulose, an activated charcoal, a glycerin, a butter, an amino acid, a paraffin, honey, a wax, beeswax, an agar, calcium carbonate, a citric acid, tartaric acid, a steric acid, xanthan gum, benzoic acid, a polyethylene glycol, a silicon, derivatives thereof, salts thereof, or any combination thereof. In some cases, a composition disclosed herein can comprise a filler, a binder, a disintegrant, a coating, a sorbent, an anti-adherent, a lubricant, a glidant, an antioxidant, a surfactant, a flavoring agent, a solvent, a buffering agent, a chelating agent, a viscosity imparting agent, a surface active agent a humectant or any combination thereof.

In some cases, a composition disclosed herein can comprise a dermal filler. In some instances, a dermal filler can be hyaluronic acid, calcium hydroxylapatite, poly-L-lactic acid, polymethylmethacrylate, autologous fat, BOTOX®, or any combination thereof.

In some cases, a composition disclosed herein can comprise a preservative. In some instances, the preservative can be an organic/natural compound, a synthetic compound or any combination thereof. In some instances, the preservative can be an antimicrobial, an antibacterial, an antifungal, an antiviral, an antiseptic, a detergent, or any combination thereof. In some cases, the preservative can be a paraben, a formaldehyde releaser, an isothiazolinone, a phenoxyethanol, an organic acid, a quaternary ammonium compound, or any combination thereof.

In some cases, a composition disclosed herein can comprise a cosmetically appropriate ingredient. In some instances, the composition may be safe under labeled or customary conditions of use. In some instances, a packaged product comprising the composition can be properly labeled, and the use of the ingredient does not otherwise cause the cosmetic to be adulterated or misbranded. In some instances, the adulterated condition can include: any poisonous or deleterious substance that can injure a user; a filthy, putrid, or decomposed substance; a cosmetic that may have been prepared, packed or held under insanitary conditions; a container that can comprise poisonous or deleterious substance which may render the contents injurious to health; or any combination thereof.

In some cases, a secretome disclosed herein can come from a stem cell. In some instances, the stem cell is not an embryonic stem cell, a mesenchymal stem cell, an adult stem cell, an induced pluripotent stem cell, a fetal cell, or any combination thereof. In some instances, the stem cell can be from an animal, such as a human. In some cases, stem cells can be grown in in vitro, such as in cell culture. In some instances, the secretome can comprise proteins that are free from an intact cell or a fragment thereof.

In some cases, a secretome can comprise monocyte chemoattractant protein (MCP-1), MCP-3, granulocyte-colony stimulating factor (G-CSF), C-X-C motif chemokine ligand 2 (CXCL2), CXCL2 (GRO), interleukin 6 (IL-6), interleukin 8 (IL-8), vascular endothelial growth factor (VEGF), platelet-derived growth factor (PDGF), PDGF-AA, PDGF-BB, PDGF-AB, a derivative of any thereof, a biologically active fragment of any thereof, or any combination thereof.

In some instances, the composition can comprise a protein (e.g., MCP-1) at a concentration of less than 1 nanogram/milliliter (ng/ml), 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 30 ng/ml, 35 ng/ml, 40 ng/ml, 45 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, or 10000 ng/ml. In some instances, the composition can comprise MCP-1 at a concentration of from about 1 ng/ml to about 100 ng/ml, from about 10 ng/ml to about 200 ng/ml, from about 10 ng/ml to about 400 ng/ml, from about 50 ng/ml to 300 ng/ml, from about 100 ng/ml to about 200 ng/ml, from about 150 ng/ml to about 400 ng/ml, from about 200 ng/ml to about 600 ng/ml, from about 400 ng/ml to about 700 ng/ml, from about 500 ng/ml to about 900 ng/ml, from about 600 ng/ml to about 1000 ng/ml, from about 900 ng/ml to about 1500 ng/ml, or from about 1000 ng/ml to about 10000 ng/ml.

In some instances, a secretome can comprise at least 0.01%, 0.1%. 1%, 1.25%, 2%, 3%, 4%, 5%, 6%, 7%, 8%, 9%, 10%, 20%, 30%, 40%, 50%, 60%, 70%, 80%, 90%, 95%, or 99% of the composition. In some instances, the secretome can comprise from about: 0.01% to about 0.1% of the composition, from about 0.01% to about 1% of the composition, from about 1% to about 2% of the composition, from about 1% to about 5% of the composition, from about 3% to about 8% of the composition, from about 5% to about 10% of the composition, from about 10% to about 20% of the composition, from about 20% to about 40% of the composition, from about 30% to about 50% of the composition, from about 50% to about 75% of the composition, from about 60% to about 90% of the composition, from about 75% to about 95% of the composition, or from about 80% to about 99% of the composition.

In some cases, a composition described herein can comprise an exosome, a liposome, a nanoparticle, or any combination thereof. In some instances, a liposome can be in a form of a nanoparticle. In some instances, a nanoparticle can comprise a liposome. In some cases, the exosome, the liposome, the nanoparticle, or any combination thereof, can comprise the secretome, a phospholipid, a protein, a hydrophilic active agent, a hydrophilic active agent, a vitamin, an inactive ingredient, or any combination thereof. The liposome can include, but may not be limited to, a unilamellar liposome, a multilamellar liposome, an archaeosome, a noisome, a novasome, a cryptosome, an emulsome, a vesosome, a nanoliposome, a nanoemulsion, or a derivative of any of these, or any combination thereof. The nanoparticle can include, but may not be limited to, a biopolymeric nanoparticle, an alginate nanoparticle, a xanthan gum nanoparticle, a cellulose nanoparticle, a lipid nanoparticle, a dendrimer, a polymeric micelle, a polyplexed, an inorganic nanoparticle, a nanocrystal, a metallic nanoparticle, a quantum dot, a protein nanoparticle, a polysaccharide nanoparticle, a derivative of any of these, or any combination thereof.

In some instances, a nanoparticle can be less than 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm in diameter. In some instances, the nanoparticle can be more than 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm in diameter. In some instances, the nanoparticles can have an average particle size of from about 1 nm to about 100 nm, from about 10 nm to about 200 nm, from about 10 nm to about 400 nm, from about 50 nm to 300 nm, from about 100 nm to about 200 nm, from about 150 nm to about 400 nm, from about 200 nm to about 600 nm, from about 400 nm to about 700 nm, from about 500 nm to about 900 nm, from about 600 nm to about 1000 nm, or from about 700 nm to about 1500 nm in diameter.

In some cases, a composition described herein can comprise a hydrophobic active agent, a fatty acid molecule, a ceramide, a phospholipid, linoleic acid, or any combination thereof. In some instances, a fatty acid can be an omega-6 polyunsaturated fatty acid, an omega-3 polyunsaturated fatty acid, linoleic acid, stearic acid, oleic acid, lauric acid, myristic acid, palmitic acid, α-linolenic acid, γ-dihomo-γ-linolenic acid, arachidonic acid, eicosatetraenoic acid, eicosapentaenoic acid, docosahexaenoic acid, a hydroxy fatty acid, a prostaglandin, a derivative or any of these, or any combination thereof. In some instances, the fatty acid, the phospholipid or any combination thereof can be comprised in an oil. In some instances, an oil can be sunflower seed oil, safflower oil, evening primrose oil, borage oil, olive oil, argan oil, jojoba oil, tea tree oil, rosemary oil, castor oil, peppermint oil, flaxseed oil, menhaden fish oil, hemp oil, shea butter, grapeseed oil, poppy seed oil, almond oil, apricot kernel oil, sesame oil, wheat germ oil, avocado oil, turtle oil, mink oil, animal oil, vegetable oil, coconut oil, an essential oil, or any combination thereof. In some instances, the phospholipid can be a saturated phospholipid, an unsaturated phospholipid, a monoacylphospholipid or any combination thereof. In some instances, the phospholipid can comprise a liposome, an exosome or any combination thereof. In some instances, the phospholipid can be phosphatidic acid (phosphatidate), phosphatidylethanolamine (cephalin), phosphatidylcholine (lecithin), phosphatidylserine, phosphoinositides, phosphatidylinositol, phosphatidylinositol phosphate, phosphatidylinositol bisphosphate, phosphatidylinositol trisphosphate, ceramide phosphorylcholine (sphingomyelin), ceramide phosphorylethanolamine (sphingomyelin), ceramide phosphoryl lipid, a derivative or any of these, or any combination thereof. In some cases, a composition can be substantially free of a steroid. In some cases, a composition can be substantially free of cholesterol. For example, the steroid can be alclometasone, amcinonide, beclomethasone, betamethasone, clobetasol, clocortolone, desonide, diflorasone, fluocinolone, hydrocortisone, halcinonide, mometasone, triamcinolone, a derivative thereof, or any combination thereof.

In some cases, a composition described herein can comprise a hydrophilic active agent, a vitamin, or any combination thereof. In some instances, a vitamin can comprise vitamin B5, provitamin B5, vitamin A, vitamin B3, vitamin C, vitamin E, a derivative thereof, a salt thereof, or any combination thereof. In some cases, the composition can comprise collagen. In some instances, collagen can comprise type I, type II, type III, type IV, type V, type VI, type VII a derivative thereof, or any combination thereof. In some cases, the composition may be substantially free from serum, an antibiotic, or a combination thereof.

### Methods of Formulation Compositions

In some aspects, a composition disclosed herein is formulated as a pharmaceutical composition. In some aspects, a composition disclosed herein is formulated as a cosmeceutical composition. In some instances, the compositions can be made by mixing secretomes and optionally one more active agents, and a pharmaceutically acceptable excipient. In some instances, the excipient comprising one or more of cellulose, disodium hydrogen phosphate, hydroxypropyl cellulose, hypromellose, lactose, mannitol, or sodium lauryl sulfate. In some instances, the compositions further comprise glyceryl monostearate 40-50, hydroxypropyl cellulose, hypromellose, magnesium stearate, methacrylic acid copolymer type C, polysorbate 80, sugar spheres, talc, or triethyl citrate. In some instances, the compositions further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, or yellow ferric oxide. In some instances, the compositions further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate. Examples of carriers for the composition include any degradable, partially degradable or non-degradable and generally biocompatible polymer, e.g., polystirex, polypropylene, polyethylene, polacrilex, poly-lactic acid (PLA), polyglycolic acid (PGA) and/or poly-lactic polyglycolic acid (PGLA), e.g., in the form or a liquid, matrix, or bead.

In some instances, a pH value of a liquid composition disclosed herein is from about 2.5 to about 5.0, 6.0 to about 8.0, from about 5.0 to about 9.0, from about 4.0 to about 10.0, from about 7.0 to about 8.0, from about 7.0 to about 9.0, from about 7.0 to about 10.0, from about 6.0 to about 7.0, from about 5.0 to about 7.0, or from about 4.0 to about 7.0. In some instances, a pH of a liquid composition disclosed herein is about 2, about 3, about 4, about 5, about 6, about 7, about 8, about 9, about 10, about 11, or about 12. In some instances, a composition disclosed herein comprises a buffer for example a phosphate buffer.

In some instances, a method for preparing a liquid composition includes blending a mixture comprising one or more active agents under conditions that minimize the introduction of air. The conditions that minimize, reduce and/or eliminate the introduction of air and/or air bubbles include one or more of the following steps used alone, in combination and/or in any order: using a diaphragm pump to combine, e.g., the water and the thixotropic agent and one or more preservatives, colorants and flavorants; placing the recirculating tube below the surface of the liquid; adding liquids along the side of a vessel holding the liquid; optionally sprinkling beads (e.g., one or more beads that includes one or more active agents) onto the surface of the liquid; mixing the solution in the absence of one or more paddles that scrape the vessel; mixing the solution with a propeller mixer; or mixing the solution with a propeller mixer at a speed that reduces or minimizes cavitation, or combinations of two or more of these steps. In another aspect, a method for preparing a liquid composition includes blending a mixture of one or more controlled-release beads/particles with one or more active agents on a carrier in a solution having a low ionic concentration and a thixotropic agent, under conditions that minimize the introduction of air bubbles.

In some instances, a liquid composition disclosed herein is a suspension comprising beads (e.g., microbeads), wherein a portion of the one or more beads have an immediate release profile and another portion have a controlled release profile. In some instances, one or more beads include an enteric coat, a resin coat, a lacquer coat, a pH-sensitive coating, a biodegradable polymer matrix, a water-soluble matrix, an ionic matrix, or any combination thereof. In some instances, one or more beads include one or more polymers selected from cellulose, ethylcellulose, methylcellulose, propylcellulose, methoxypropylcellulose, cellulose nitrate, poly(vinyl alcohol), poly(vinyl chloride), polystyrene, polyethylene, polypropylene, poly(ethylene-co-vinyl acetate), poly(hydroxybutyric acid), poly(hydroxyvalerianic acid-co-hydroxybutyric acid), poly(lactic acid), poly(glycolic acid), poly(lactic acid-co-glycolic acid), poly(epsilon(-caprolactones), poly(epsilon-caprolactone-co-DL-lactic acid), poly(maleic anhydride), polyamides, gelatin, chitosan, collagen, poly(hydroxyalkyl)-L-glutamines, poly(gamma-ethyl-L-glutaminate-co-glutamic acid), poly(L-leucine-co-L-aspartic acid), poly(proline-co-glutamic acid), poly(alkyl 2-cyanoacrylates), polyurethanes, poly(methyl methacrylate), poly(methyl methacrylate-co-methacrylic acid) and poly(methacrylate-co-hydroxypropyl methacrylate), polystyrene, polistirex, polacrilex, salts thereof, and any combination thereof.

In some instances, compositions disclosed herein are in unit-dosage forms or multiple-dosage forms. Unit-dosage forms, as used herein, refer to physically discrete units suitable for administration to human or non-human animal subjects and packaged individually. Each unit-dose contains a predetermined quantity of an active ingredient(s) sufficient to produce the desired therapeutic effect, in association with the required pharmaceutical carriers or excipients. Examples of unit-dosage forms include, but are not limited to, ampules, syringes, and individually packaged tablets and capsules. In some instances, unit-dosage forms are administered in fractions or multiples thereof. A multiple-dosage form is a plurality of identical unit-dosage forms packaged in a single container, which is administered in segregated unit-dosage form. Examples of multiple-dosage forms include, but are not limited to, vials, bottles of tablets or capsules, or bottles of pints or gallons. In some instances, the multiple dosage forms comprise different pharmaceutically active agents.

In some aspects, a composition disclosed herein can have a liposome, which can be prepared in variety of methods that are acceptable for the composition. In some cases, the liposome preparation can include drying the lipids, dispersing the lipids, purification of liposomes and analysis of the final product. In some instances, liposomes can be prepared through methods comprising: sonication, ultrasonication, a French pressure cell, extrusion, membrane extrusion, lipid film hydration or any combination thereof. In some instances, nanoliposomes can be prepared from liposomes by reducing the particle size using high pressure homogenization, ultrasound, or membrane extrusion. In some instances, the nanoemulsion can be formed by mixing oil, an emulsifier and water. In some instances, an oil-in-water or a water-in-oil nanoemulsion can be formed.

In some aspects, secretome proteins, for example, cytokines, chemokines, or any combination thereof, can be present in a free form (soluble), on surface of exosomes (surface-bound), encapsulated within an exosome, or any combination thereof. Exosome may be contained in the composition in the form of a liposome encapsulating the exosome by encapsulating the exosome into the liposome. In some cases, the exosome can be in any form as long as it is suitable for use as a composition. In some instances, the exosome may be used without being encapsulated into a liposome. In some instances, when the exosome is used in the form of liposome encapsulation, the exosome may be contained in an amount of about 0.1% to about 10.0% by weight, or in an amount of about 0.1% to about 1.0% by weight based on the total weight of the liposome. In some instances, the liposome encapsulating the exosome may be contained in an amount of about 0.001% to about 10.0% by weight, about 0.001% to about 1.0% by weight, about 0.01% to about 1.0% by weight, or about 0.01% to about 0.1% by weight based on the total weight of the entire composition.

In some cases, about 3% by weight of lecithin can be dispersed in an aqueous phase containing about 0.01% by weight of the exosomes derived from stem cells (e.g., about 15° C.), and then a reverse micelle emulsion (water and low temperature process carbon dioxide) can be formed using supercritical carbon dioxide. In some instances, the reaction can be terminated, and the supercritical carbon dioxide can be vaporized under reduced pressure to remove the supercritical carbon dioxide phase, thereby obtaining a low-temperature process liposome suspension in which the exosomes can be encapsulated. In some instances, the composition can be prepared such that the liposome encapsulating the exosomes can be contained in an amount of about 5% by weight based on the total weight of the entire composition.

In some cases, nanoliposomes can be made from a precursor solution. In some instances, a precursor solution may be made by solubilizing an amphipathic material in a first quantity of a non-aqueous solvent appropriate to solubilize the amphipathic material to form a first mixture. The amphipathic material can comprise phospholipids (PL). A PL can comprise one or more of the following phosphatides: phospatidylcholine (PC), phospatidylethanolamine (PE), phosphatidic acid (PA) and phosphatidylinositol (PI). In some cases, PC, PE, PA and PI are combined. In some instances, a ratio of PLs useful may be PC:PE:PA:PI of about 6.5:2.5:0.7:0.3 in ethanol. In some instances, one gram of PL is solubilized in 5.0 ml to 7.5 ml of ethanol solvent. In some instances, after dissolution of the amphipathic material, a quantity of water can be added to form a turbid suspension. In some instances, the amount of water to add can be from about 9 kg to about 31 kg of dissolved amphipathic material but can be varied to result in the desired turbid suspension. In some instances, a second quantity of non-aqueous solvent, such as ethanol, can be added until the turbid suspension is monophasic and has optical clarity at room temperature. In some instances, the resulting product can be a precursor solution which may be shelf-stable over time. In some cases, precursor solutions made by this method can be stable for at least about: 1 year, 2 years, 3 years, 4 years, 5 years, 6 years, 8 years, or 9 years, independent of manufacturing, location, season, year, and/or lot. In some instances, a precursor solution can be used as a starting material to make nanoliposome and nanoliposome assemblies. In some cases, the precursor solution can be useful for making an amphipathic carrier structure denoted as a Solvent Dilution Microcarrier (SDMC). In some instances, the SDMC can have a diameter of from about 230 to about 412 nm. In some instances, nanoliposomes can have a mean diameter of from about 1 nm to about 20 nm and nanoliposome assemblies can have a mean diameter from about 30 nm to about 200 nm.

In some cases, to make carriers for passenger molecules, such as nanoliposome populations, nanoliposome assemblies, or mixed population lysosomes, a precursor solution can be diluted with a suitable solvent or mixed solvent system which can be compatible with the solvent system used in the precursor solution. In some instances, this dilution can be performed either before or after addition of the passenger molecule. The solvent can be selected for biocompatibility if the end use of the carriers may require that characteristic. In some instances, the solvent or mixed solvent system used for dilution may be miscible with the solvents in the precursor solution and can be effective to disperse the carriers. In some instances, the solvent used for dilution can be ethanol. In some instances, the dilution can be conducted in a sequential or serial manner. For example, a first dilution of about 1:10 provides a population of carriers, and a further serial dilution to about 1:0.5 provides a series of populations of carriers. The size of the carriers in each dilution can be determined by laser light scattering. In some instances, mixed populations of nanoliposomes and larger vesicles may be created at lower dilutions with the non-aqueous solvent. An appropriate instrument for this purpose can be the ZETASIZER® 1000 manufactured by Malvern Instruments, (Worcestershire United Kingdom). Diameters of particles reported herein were determining using the Multimodal Analysis Mode of the ZETASIZER® 1000 to determine particle size by peak intensities. In some instances, other techniques may be used to analyze particle size, which results can be correlated to the numerical values obtained with the light scattering technique. In some instances, addition of the desired passenger molecule can occur prior to dilution with the solvent if the passenger molecule is lipophilic or amphipathic. In some instances, addition occurs after dilution if the passenger molecule is water soluble. In some instances, in the case of a lipophilic or amphipathic passenger molecule, the nanoliposome loaded populations can form upon dilution with the solvent. In some instances, nanoliposome assembly populations or mixed population liposomes can be formed by dilution of the nanoliposome loaded population into water.

### Methods of Use

In some aspects, disclosed herein is a method, comprising administering a composition disclosed herein to a subject (e.g., a human) in need thereof. In some instances, the method treats a disease in the subject. In some instances, the method ameliorates a condition of the subject’ skin.

In some cases, a composition herein can be used to treat a disease. In some instances, the composition herein can be used to facilitate autocrine, juxtacrine, and paracrine effects. In some instances, the administration comprises injecting the composition, e.g., intravenously, intramuscularly, or subcutaneously. In some instances, a composition can be applied directly to the treatment site, for example, a topical treatment applied to a wound. In some instances, a composition herein can be used as a preventative medicine, e.g., from aging of a tissue or an organ. In some instances, a composition herein can be used as a regenerative medicine. In some instances, a composition herein can be used for tissue repair to treat various conditions rising from injury or damage of tissue, organ, or any combination thereof. In some instances, a composition herein treats or prevents stroke, alopecia, baldness, cartilage defect, myocardial infarct, hindlimb ischemia, spinal cord injury, nerve injury, lung injury, bone defect, intrabony periodontal defect, periodontal disease, skin wound, cerebral injury, traumatic brain injury, liver failure, graft versus host disease (GVHD), or any combination thereof. In some instances, a composition herein can be used to treat a chronic disease. In some instances, the chronic disease can be a kidney disease, liver disease, or any combination thereof.

In some aspects, disclosed herein is a method for modulating a skin condition. In some instances, the methods can be used to treat a skin disease or condition. In some instances, the disease or condition is eczema, rash, psoriasis, acne, rosacea, ichthyosis, vitiligo, hive, seborrheic dermatitis, shingles, burn, sunburn, contact dermatitis, wrinkled skin, scarred skin, sagging skin, loss of skin elasticity, skin dryness, skin dullness, or any combination thereof. In some instances, the method can reduce the appearance of skin aging, photoaging, or any combination thereof. In some instances, the method can reduce the appearance of a scar. In some instances, the method can improve wound healing. In some instances, the method can prevent, reduce or eliminate bruising, benign growths, age spots, cancerous growths, ulcers, infections, or any combination thereof. In some instances, the method can prevent, reduce, or eliminate lines, wrinkles, or any combination thereof of skin. In some instances, the lines or wrinkles can be crow’s feet, smile lines, frown lines, forehead furrows, tear troughs, bunny lines, nasolabial folds, marionette lines, mental crease, necklines, age-related wrinkles, crinkle lines, elastotic creases, expression lines, gravitational folds, dynamic wrinkles, static wrinkles, atrophic wrinkles, atrophic crinkling rhytids, or any combination thereof. In some instances, the method can prevent, reduce or eliminate loss of volume, elasticity, or any combination thereof of skin. In some instances, the method can prevent, reduce or eliminate, sagging skin, dull skin tone, mottled discoloration, rough skin, dry skin, itchy skin, thin skin, or any combination thereof.

In some cases, the method can improve or ameliorate a skin condition, skin disease or any combination thereof. In some instances, the method can moisturize, tighten, lift, or rejuvenate skin. In some instances, the method can restore or sustain a healthy, smooth, blemish-free, translucent, resilient, or any combination thereof skin. In some cases, the method can heal, treat, remedy or any combination thereof the glycosaminoglycan, the dermis, the collagen and the elastin of skin. In some cases, the improved health of skin can be measured by a wrinkle severity rating scale, a trans-epidermal water loss measurement, a skin color measurement, a skin surface topography measurement, a viscoelastic measurement by a CUTOMETER®, a histological examination, or any combination thereof. In some cases, improved skin health can be measured by a diagnostic image, such as magnetic resonance imaging (MRI). In some cases, measurements can be compared before and after administration of the composition. In some instances, measurements can be compared to a standard.

In some cases, administration of the composition can include topical or dermal administration. In some instances, a topical dosage form can be a lotion, a solution, an emulsion, a paste, a suspension, a tablet, a stick, an aerosol (e.g., spray, puff, or foam), a butter, an oil, a cream, a patch, a gel (e.g., a hydrogel), a milk or milky form, a spray, a drip, a liquid, a powder, a solid, an ointment, a bead, a mask, a pad (e.g., an impregnated pad), a sheet, dispersion, microemulsion, or any combination thereof. In some instances, the composition can be applied by pouring, sprinkling, spraying, rubbing, introduced onto, or otherwise. In some instances, topical administration can be administered directly to the site of the condition. In some instances, a patch can comprise a membrane, a microneedle patch, a single-layer cosmetic in adhesive, a multi-layer cosmetic in adhesive, a reservoir system, a matrix system, or any combination thereof. In some instances, the composition herein can be a dosage form such as wound dressing, bandage (e.g., coated bandage or other polymer covering), ointment, cream, lotion, pasts, jelly, spray, or aerosol.

In some cases, administration of the composition can include an injection. In some instances, an injection can comprise administering an injector, subcutaneous injection, intradermal injection, a dermal injection, intravenous injection, intraarterial injection, intramuscular injection, intraorbital injection, intraperitoneal injection, intravenous injection, intraventricular injection, stereotactic injection, or any combination thereof. In some instances, an injection can be administered directly to the site of the condition.

In some instances, a subject can administer the composition in the absence of supervision. In some instances, the cosmetic can be administered by another person, a nurse, a clinician, a physician, a cosmetic professional, a beautician, a medical professional, or any combination thereof.

### Methods of Making Therapeutic Compositions Under Hypoxic Conditions

In one aspect, provided herein is a method of producing one or more proteins of interest from a trophoblastic cell line, the method comprising: culturing human trophoblastic stem cells with a nutritional media until confluency is reached; inducing hypoxia; and isolating the one or more proteins of interest from the media. In some instances, hypoxia is induced for approximately 12-48 hours and, in some instances, is induced for about 24 hours. Confluency can vary depending upon the culture dish being utilized. Non-limiting examples of confluency can comprise from about 3,000 cells/cm² to about 9,000 cells/cm², from about 4,000 cells/cm² to about 8,000 cells/cm², from about 5,000 cells/cm² to about 7,000 cells/cm², or about 6,000 cells/cm². The method of any one of claims 70-72, wherein the isolated one or more proteins can, optionally, be further mixed with one or more pharmaceutically acceptable excipients in order to prepare a composition.

The described method can produce one or more proteins that can comprise a cytokine, a growth factor, a membrane-bound signaling molecule, a cell adhesion molecule, a defense protein, an immunity protein, and extracellular matrix protein, an intracellular signal molecule, a metabolite interconversion enzyme, a protein modifying enzyme /protease, a protein-binding modulator/protease inhibitor, a scaffold/adaptor protein, a structural protein, a transfer protein or a carrier protein, a transmembrane signal receptor, or any combination thereof. A secretome produced from any one of the described methods is useful in one or more of the following processes: biological adhesion/cell adhesion, biological regulation, cell proliferation, cellular component organization or biogenesis, a cellular process (e.g., cell activation, cell communication, cell cycle process, cell death, cell growth, cellular component organization, cellular developmental process, cell differentiation, cellular component morphogenesis, cellular metabolic process, cellular response to stimulus, export from cell, microtubule-based process, cell activation, cell communication, cell cycle process, cell death, cell growth, cellular component organization, cellular developmental process (e.g., cell differentiation, or cellular component morphogenesis), cellular metabolic process, cellular response to stimulus, export from cell, microtubule-based process, movement of cell or subcellular component, cell motility, neuron projection guidance, myelination, signal transduction, etc.), developmental process (e.g., anatomical structure development, anatomical structure formation involved in morphogenesis, anatomical structure morphogenesis, cellular developmental process, developmental growth, etc.), growth, an immune system process (e.g., immune effector process, immune response, immune system development, leukocyte activation, leukocyte migration, etc.), localization (e.g., cellular localization, establishment of localization, localization of cell, macromolecule localization, etc.), a locomotion (e.g., cell motility, taxis, etc.) a metabolic processes (e.g., biosynthetic process, catabolic process, cellular metabolic process, hormone metabolic process, nitrogen compound metabolic process, etc.), a multi-organism process / response to other organism, a multicellular organism process (e.g., coagulation, cytokine production, digestion, multicellular organism development, system process, etc.), a response to stimulus (e.g., cellular response to stimulus, immune response, etc.), signaling (e.g., cell-cell signaling, signal transduction, etc.).

A secretome produced from any one of the described methods is useful in one or more of the following pathways: Alzheimer disease-amyloid secretase pathway; Alzheimer disease-presenilin pathway; angiogenesis; apoptosis signaling pathway; axon guidance mediated by Slit/Robo; Axon guidance mediated by netrin; blood coagulation; CCKR signaling map; cadherin signaling pathway; endothelin signaling pathway; FAS signaling pathway; gonadotropin-releasing hormone receptor pathway; inflammation mediated by chemokine and cytokine signaling pathway; insulin/IGF pathway - MAPKK/MAPK cascade; insulin/IGF pathway - PKB signaling cascade; interleukin signaling pathway; PDGF signaling pathway; plasminogen activating cascade; T cell activation; TGF-beta signaling pathway; toll receptor signaling pathway; Wnt signaling pathway; P53 pathway, etc.

In one instance, the method produces one or more proteins that comprise Chemokine (C-C motif) ligand 13 (CCL13), CCL20, CCL25, CCL26, CCL28, CCL4, CCL5, CCL7, CCL8, C-X-C Motif Chemokine Ligand 1 (CXCL1), CXCL11, CXCL12, CXCL14, CXCL15, Pf4, or any combination thereof. In another instance, the method produces one or more proteins that comprise Secreted Phosphoprotein 1 (SPP1), DKK1, Serpin Family E Member 1 (SERPINE1), FLT1, Follistatin Like 3 (FSTL3), Matrilin 3 (MATN3), Pregnancy-associated plasma protein A (PAPPA), Growth Differentiation Factor 15 (GDF15), human growth factor (HGF), Insulin Like Growth Factor Binding Protein 3 (IGFBP3), or any combination thereof. In another instance, the method produces one or more proteins that comprise FST, Nidogen 1 (NID1), MET (MET Proto-Oncogene, Receptor Tyrosine Kinase), TGFβI, Follistatin Like 1 (FSTL1), Nidogen 2 (NID2), Cysteine-rich motor neuron 1 (CRIM1), Platelet Derived Growth Factor Subunit B (PDGFB), or any combination thereof. In another instance, the method produces one or more proteins that comprise CXCL12, Galectin 1 (LGALS1), ADAMTS Like 1 (ADAMTSL1), or any combination thereof. In another instance, the method produces one or more proteins that comprise FAP, IGFBP3, or a combination thereof. In another instance, the method produces one or more proteins that comprise CCL13, CCL20, CCL25, CCL26, CCL28, CCL4, CCL5, CCL7, CCL8, CXCL1, CXCL11, CXCL12, CXCL14, CXCL5, Platelet Factor 4 (PF4), or a combination thereof. In another instance, the method produces one or more proteins that comprise Colony Stimulating Factor 1 (CSF1), Growth Differentiation Factor 15 (GDF15), Interferon Lambda 1 (IFNL1), Interferon Lambda 2 (IFNL2), interleukin 21 (IL21), IL6, macrophage inhibitory factor (MIF), Nicotinamide phosphoribosyltransferase (NAMPT), SPP1, TGFβ1, TIMP Metallopeptidase Inhibitor 1 (TIMP1), or a combination thereof. In another instance, the method produces one or more proteins that comprise CSF1, CXCL12, DKK1, GDF15, HGF, IL6, PDGFB, TGFB1, TIMP1, or a combination thereof. In another instance, the method produces one or more proteins that comprise ANG, CSTB, NAP1L4, toll-like receptor 3 (TLR3), or a combination thereof. In another instance, the method produces one or more proteins that comprise ADAMTSL1, DCN, FURIN, Lumican (LUM), Matrilin 3 (MATN3), matrix metalloproteinase 1 (MMP1), NID1, NID2, PDGFB, Periostin (POSTN), Pentraxin 3 (PTX3), SERPINE, SPP1, TGFβI, Thrombospondin 1 (THBS1), TIMP1, TIMP2, CCN1, LUM, MATN3, NID1, NID2, POSTN, or a combination thereof.

In another instance, the method produces one or more proteins that comprise ANG, Beta-2-Microglobulin (B2M), BCL10, CCL13, CCL20, CCL25, CCL28, CCL4, CD99, CLU, CSF1, CXCL1, CXCL1, CXCL11, CXCL12, CXCL14, CXCL5, F11 Receptor (F11R), Fas Cell Surface Death Receptor (FAS), IFNL1, IFNL2, IL21, IL6, IL7R, LGALS3, MIF, Osteoclast Associated Ig-Like Receptor (OSCAR), PF4, PTX3, SERPINE1, Sialic Acid Binding Ig Like Lectin 9 (SIGLEC9), THBS1, TLR3, TNF Receptor Superfamily Member 21 (TNFRSF21), or a combination thereof. In another instance, the method produces one or more proteins that comprise CCL13, CCL13, CCL20, CCL25, CCL4, CCL5, CCL7, CCL8, CSF1, CXCL1, CXCL12, F11R, IGFBP4, IL6, MIF, nucleoporin 85 (NUP85), PF4, PTX3, Semaphorin 7A (SEMA7A), SPP1, THBS1, TNFRSF1A, or a combination thereof. The method of any one of claims 70-73, wherein the one or more proteins comprise ANG, CCL13, CCL20, CCL25, CCL26, CCL8, CLU, CXCL1, CXCL11, CXCL12, CXCL14, CXCL5, LGALS3, PF4, ANG, B2M, CCL20, KLK3, TLR3, TNFRSF1A, CCL4, IFNL1, IFNL2, IL21, IL6, TLR3, or a combination thereof. In another instance, the method produces one or more proteins that comprise ANG, B2M, CCL20, KLK3, TLR3, TNFRSF1A, or any combination thereof. In another instance, the method produces one or more proteins that comprise CCL4, IFNL1, IFNL2, IL21, IL6, TLR3, or any combination thereof. In another instance, the method produces one or more proteins that comprise DCN, POSTN, Syndecan-4 (SDC4), granulin (GRN), PAPPA, TIMP1, or a combination thereof. In another instance, the method produces one or more proteins that comprise Angiopoietin 1 (ANGPT1), Fms Related Receptor Tyrosine Kinase 1 (FLT1), MET, Cystatin C (CST3), Dickkopf-related protein 3 (DKK3), Retinol Binding Protein 4 (RBP4), Basigin (BSG), or a combination thereof. In another instance, the method produces one or more proteins that comprise ANG, Decorin (DCN), Baculoviral IAP Repeat Containing 2 (BIRC2), Platelet Derived Growth Factor Subunit B (PDGFB), or a combination thereof. In another instance, the method produces one or more proteins that comprise tissue inhibitor of metalloproteinases 4 (TIMP4), CRIM1, Unc-5 Netrin Receptor C (UNC5C), tissue inhibitor of metalloproteinases 2 (TIMP2), or a combination thereof. In another instance, the method produces one or more proteins that comprise Dickkopf-related protein 1 (DKK1), Follistatin (FST), Transforming growth factor β (TGFβ1), FLT1, DKK3, Cellular Communication Network Factor 1 (CCN1), or a combination thereof.

In another instance, the method produces one or more proteins that comprise NAP1L4, SPP1, ANGPT1, FST, MET, CTSB, FSTL1, LGALS1, Tripeptidyl Peptidase 1 (TPP1), OSCAR, CCN1, IGFBP3, TGFB1, B2M, IL7R, carboxylesterase 1 (CES1), Colony Stimulating Factor 1 (CSF1), or any combination thereof. In another instance, the method produces one or more proteins that comprise SPP1, OSCAR, CCN1, IGFBP3, or any combination thereof. In another instance, the method produces one or more proteins that comprise CSF1, MET, CCN1, TGFβ1, or LGALS 1. In another instance, the method produces one or more proteins that comprise B2M, IL7R, or a combination thereof. In another instance, the method produces one or more proteins that comprise CTSB, TPP1, CES1, or any combination thereof. In another instance, the method produces one or more proteins that comprise SIGLEC9, POSTN, TGFβI, or a combination thereof. In another instance, the method produces one or more proteins that comprise OSCAR, B2M, or a combination thereof. In another instance, the method produces one or more proteins that comprise LGALS3, LGALS1, or a combination thereof. In another instance, the method produces one or more proteins that comprise X-C Motif Chemokine Ligand 1 (XCL1), TGFB1, CCL5, CCL20, CCL28, CCL4, CXCL5, ANGPTL4, PDGFB, CCL13, CCL8, ANGPTI, CCL25, CXCL11, CCL7, PF4, GDF15, CCL26, SEMA7A, SPP1, CXCL1, or a combination thereof. In another instance, the method produces one or more proteins that comprise XCL1, CCL5, CCL20, CCL28, CCL4, CXCL5, CCL13, CCL8, CCL25, CXCL11, CCL7, PF4, CCL26, SPP1, CXCL1, or a combination thereof. In another instance, the method produces one or more proteins that comprise TGFβI, PDGFB, GDF15, or a combination thereof. In another instance, the method produces one or more proteins that comprise SEMA7A.

In another instance, the method produces one or more proteins that comprise CES1, FAS, MIF, NAMPT, SPP1, or a combination thereof. In another instance, the method produces one or more proteins that comprise Fibroblast Activation Protein Alpha (FAP), Legumain (LGMN), HGF, Cathepsin B (CTSB), TPP1, Kallikrein Related Peptidase 3 (KLK3), FURIN, MMP1, or a combination thereof. In another instance, the method produces one or more proteins that comprise IGFBP3, TIMP4, FSTL1, BIRC2, FST, SERPINE1, TIMP1, IGFBP2, FSTL3, IGFBP4, TIMP2, or a combination thereof. In another instance, the method produces one or more proteins that comprise BSG, NUP85, or low-density lipoprotein receptor (LDLR). In another instance, the method produces one or more proteins that comprise albumin (ALB), Tripeptidyl Peptidase 1 (TPP1), LDLR, Retinol Binding Protein 4 (RBP4), transferrin (TF), or a combination thereof. In another instance, the method produces one or more proteins that comprise UNC5C, TLR3, Plasminogen Activator, Urokinase Receptor (PLAUR), Glycoprotein Ib Platelet Subunit Alpha (GP1BA), SDC4, Thrombomodulin (THBD), IL7R, TF, or a combination thereof. In another instance, the method produces one or more proteins that comprise SIGLEC9, CD99, TNF Receptor Superfamily Member 21 (TNFRSF21), GP1BA, BSG, POSTN, TGFβI, or a combination thereof. In another instance, the method produces one or more proteins that comprise XCL1, IGFBP3, TGFβ1, CCL5, TIMP4, CCL20, FSTL1, CCL4, BIRC2, BCL10, CD99, LGALS3, CXCL5, TNFRSF21, FST, SERPINE1, GP1BA, PDGFB, F11R, CCL13, TIMP1, NID1, CCL8, NUP85, IGFBP6, THBS1, CCL25, IGFBP2, FSTL3, IGFBP4, KLK3, CXCL11, GPC1, CCL7, DKK3, PF4, GDF15, TF, CCL26, TIMP2, SEMA7A, FURIN, DKK1, INFRSF10C, CXCL1, or a combination thereof. In another instance, the method produces one or more proteins that comprise TGFβ1, TNFRSF21, PDGFB, or a combination thereof.

In another instance, the method produces one or more proteins that comprise UNC5C, BIRC2, LGALS3, BSG, POSTN, TGFβI, SEMA7A, MMP1, or a combination thereof. In another instance, the method produces one or more proteins that comprise TNFRSF21, GP1BA, or a combination thereof. In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise BIRC2, BCL10, LGALS3, TNFRSF21, INFRSF10C, or a combination thereof. In another instance, the method produces one or more proteins that comprise SEMA7A. In another instance, the method produces one or more proteins that comprise UNC5C, BIRC2, LGALS3, BSG, POSTN, TGFβI, SEMA7A, MMP1, or a combination thereof. In another instance, the method produces one or more proteins that comprise UNC5C, FSTL1, FST, MET, F11R, BSG, FLT1, FSTL3, SEMA7A, or a combination thereof. In another instance, the method produces one or more proteins that comprise UNC5C, FSTL1, FST, MET, F11R, BSG, FLT1, FSTL3, SEMA7A, or a combination thereof. In another instance, the method produces one or more proteins that comprise UNC5C, BSG, SEMA7A, or a combination thereof. In another instance, the method produces one or more proteins that comprise XCL1, TGFβ1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, CCL26, TIMP2, SEMA7A, FURIN, or a combination thereof.

In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise TNFRSF21. In another instance, the method produces one or more proteins that comprise TNFRSF21, GP1BA, or a combination thereof. In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise BIRC2, BCL10, LGALS3, TNFRSF21, INFRSF10C, or a combination thereof. In another instance, the method produces one or more proteins that comprise UNC5C, BIRC2, LGALS3, BSG, POSTN, TGFβI, SEMA7A, MMP1, or a combination thereof. In another instance, the method produces one or more proteins that comprise UNC5C, FSTL1, FST, MET, F11R, BSG, FLT1, FSTL3, SEMA7A, or a combination thereof. In another instance, the method produces one or more proteins that comprise UNC5C, FSTL1, FST, MET, F11R, BSG, FLT1, FSTL3, SEMA7A, or a combination thereof. In another instance, the method produces one or more proteins that comprise UNC5C, BSG, SEMAT7A, or a combination thereof. In another instance, the method produces one or more proteins that comprise XCL1, TGFβ1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, CCL26, TIMP2, SEMA7A, FURIN, or a combination thereof. In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise XCL1, CCL5, CCL20, CCL4, CD99, LGALS3, CXCL5, MET, PDGFB, CCL13, SDC4, CCL8, CCL25, CXCL11, GPC1, CCL7, PF4, CCL26, SEMA7A, CXCL1, or a combination thereof.

In another instance, the method produces one or more proteins that comprise UNC5C, BSG, SEMAT7A, or a combination thereof. In another instance, the method produces one or more proteins that comprise TNFRSF21. In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise UNC5C, FSTL1, PTX3, TNFRSF21, FST, MET, FUR, BSG, THBS1, FLT1, FSTL3, SEMA7A, or a combination thereof. In another instance, the method produces one or more proteins that comprise UNC5C, FSTL1, PTX3, TNFRSF21, FST, MET, FUR, BSG, THBS1, FLT1, FSTL3, SEMA7A, or a combination thereof. In another instance, the method produces one or more proteins that comprise THBS1.

In another instance, the method produces one or more proteins that comprise UNC5C, PTX3, BSG, THBS1, SEMA7A, or a combination thereof. In another instance, the method produces one or more proteins that comprise UNC5C, FSTL1, FST, MET, F11R, BSG, FLT1, FSTL3, SEMAT7A, or a combination thereof. In another instance, the method produces one or more proteins that comprise XCL1, CCL5, CCL20, CCL4, BCL10, CD99, LGALS3, CXCL5, TNFRSF21, PTX3, CCL13, IFNL1, CCL8, FLT1, CCL25, CXCL11, CCL7, PF4, CCL26, SEMA7A, CXCL1, or a combination thereof. In another instance, the method produces one or more proteins that comprise IFNL1, SEMA7A, or a combination thereof. In another instance, the method produces one or more proteins that comprise XCL1, CCL5, CCL20, CCL4, BCL10, CXCL5, TNFRSF21, PTX3, CCL13, IFNL1, CCL8, FLT1, CCL25, CXCL11, CCL7, PF4, CCL26, SEMAT7A, CXCL1, or a combination thereof. In another instance, the method produces one or more proteins that comprise FLT1. In another instance, the method produces one or more proteins that comprise TNFRSF21. In another instance, the method produces one or more proteins that comprise XCL1, CCL5, CCL20, CCL4, BCL10, CD99, LGALS3, CXCL5, CCL13, CCL8, CXCL11, CCL7, PF4, CCL26, CXCL1, or a combination thereof. In another instance, the method produces one or more proteins that comprise NUP85, GPC1, or a combination thereof. In another instance, the method produces one or more proteins that comprise ALB, LGALS3, TNFRSF21, MET, F11R, NUP85, or a combination thereof. In another instance, the method produces one or more proteins that comprise XCL1, CCL5, CCL20, CCL4, CD99, LGALS3, CXCL5, MET, PDGFB, CCL13, SDC4, CCL8, CCL25, CXCL11, GPC1, CCL7, PF4, CCL26, SEMA7A, CXCL1, or a combination thereof. In another instance, the method produces one or more proteins that comprise TNFRSF21, NUP85, GPC1, or a combination thereof.

In another instance, the method produces one or more proteins that comprise XCL1, CCL5, CCL20, CCL4, CD99, LGALS3, CXCL5, MET, PDGFB, CCL13, SDC4, CCL8, or a combination thereof. In another instance, the method produces one or more proteins that comprise CCL25, CXCL11, GPC1, CCL7, PF4, CCL26, SEMA7A, CXCL1, or a combination thereof. In another instance, the method produces one or more proteins that comprise XCL1, UNC5C, CCL5, CCL20, CCL4, LGALS3, CXCL5,, CCL13, BSG, CCL8, CCL25, CXCL11, CCL7, PF4, CCL26, SEMA7A, CXCL1, or a combination thereof. In another instance, the method produces one or more proteins that comprise TGFβ1, FSTL1, BCL10, FST, NUP85, FSTL3, GDF15, or a combination thereof. In another instance, the method produces one or more proteins that comprise TIMP4, LGMN, CED1, TIMP1, CTSB, TIMP2, MMP1, or a combination thereof. In another instance, the method produces one or more proteins that comprise XCL1, TGFβ1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, CCL26, TIMP2, SEMA7A, FURIN, or a combination thereof. In another instance, the method produces one or more proteins that comprise FURIN. In another instance, the method produces one or more proteins that comprise XCL1, TGFβ1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, or a combination thereof. In another instance, the method produces one or more proteins that comprise CCL26, TIMP2, SEMA7A, FURIN, or a combination thereof. In another instance, the method produces one or more proteins that comprise XCL1, CCL5, CCL20, CCL4, CXCL5, PTX3, CCL13, IFNL1, CCL8, CCL25, CXCL11, CCL7, PF4, CCL26, CXCL1, or a combination thereof.

In another instance, the method produces one or more proteins that comprise GP1BA. In another instance, the method produces one or more proteins that comprise TNFRSF21, SEMA7A, or a combination thereof. In another instance, the method produces one or more proteins that comprise UNC5C, FSTL1, TNFRSF21, FST, MET, BSG, THBS1, FLT1, FSTL3, SEMAT7A, or a combination thereof. In another instance, the method produces one or more proteins that comprise TNFRSF21, FUR, KLK3, or a combination thereof. In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise XCL1, CCL5, CCL20, CCL4, BCL10, CXCL5, TNFRSF21, PTX3, CCL13, IFNL1, CCL8, CCL25, CXCL11, CCL7, PF4, CCL26, SEMA7A, CXCL1, or a combination thereof. In another instance, the method produces one or more proteins that comprise NID1, DKK1, DKK3, or a combination thereof. In another instance, the method produces one or more proteins that comprise XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof. In another instance, the method produces one or more proteins that comprise FSTL1, FURIN, MMP1, or a combination thereof. In another instance, the method produces one or more proteins that comprise PDGFB, ANGPT1, TF, or a combination thereof. In another instance, the method produces one or more proteins that comprise BIRC2, FAS, TNFRSF1A, INFRSF10C, or a combination thereof.

In another instance, the method produces one or more proteins that comprise CXCL12. In another instance, the method produces one or more proteins that comprise UNC5C. In another instance, the method produces one or more proteins that comprise SERPINE1, PLAUR, GP1BA, THBD, KLK3, TF, or a combination thereof. In another instance, the method produces one or more proteins that comprise BIRC2, SERPINE1, CLU, CXCL1, or a combination thereof. In another instance, the method produces one or more proteins that comprise FSTL1. In another instance, the method produces one or more proteins that comprise FAS. In another instance, the method produces one or more proteins that comprise TGFβ1, Macrophage Migration Inhibitory Factor (MIF), Follistatin (FST), insulin (INS), or a combination thereof. In another instance, the method produces one or more proteins that comprise IL6, CCL5, CCL20, CCL4, CCL13, CCL8, CCL7, PF4, CCL26, or a combination thereof. In another instance, the method produces one or more proteins that comprise insulin (INS). In another instance, the method produces one or more proteins that comprise IL6, IL21, or a combination thereof. In another instance, the method produces one or more proteins that comprise platelet derived growth factor beta (PDGFβ). In another instance, the method produces one or more proteins that comprise SEREPIN1, PLAUR, MMP1, or a combination thereof. In another instance, the method produces one or more proteins that comprise β2 microglobulin (B2M).

In another instance, the method produces one or more proteins that comprise TGFβ1, GDF15, or a combination thereof. In another instance, the method produces one or more proteins that comprise toll-like receptor 3 (TLR3). In another instance, the method produces one or more proteins that comprise Follistatin Like 1 (FSTL1). In another instance, the method produces one or more proteins that comprise IGFBP3, SEREPINE1, FAS, THBS1, or a combination thereof. In another instance, the method produces one or more proteins that comprise f11R.

### Exemplary Compositions and Therapeutic Uses Thereof or In Vitro/Ex Vivo Use

Provided herein is a composition that comprises a) about 0.1% or more w/w of a secretome and b) a pharmaceutically acceptable excipient, wherein the secretome comprises one or more of CCL13, CCL20, CCL25, CCL26, CCL28, CCL4, CCL5, CCL7, CCL8, CXCL1, CXCL11, CXCL12, CXCL14, CXCL15, PF4, or any combination thereof. In one instance, the secretome comprises one, two, three, four, five, six, seven, eight, nine, ten, eleven, twelve, thirteen, or fourteen of CCL13, CCL20, CCL25, CCL26, CCL28, CCL4, CCL5, CCL7, CCL8, CXCL1, CXCL11, CXCL12, CXCL14, CXCL15, and PF4. In another instance, the secretome comprises CCL13, CCL20, CCL25, CCL26, CCL28, CCL4, CCL5, CCL7, CCL8, CXCL1, CXCL11, CXCL12, CXCL14, CXCL15, and PF4. Provided herein is a method of inducing chemokine activity in a subject, comprising administering to a subject in need thereof any of such compositions, wherein the composition induces chemokine activity in the subject. Provided herein is a use of any of such compositions, for treating a subject in need thereof, wherein the composition induces chemokine activity in the subject. Provided herein is a use of any of such compositions, for the manufacture of a medicament for treating a subject in need thereof, wherein the composition induces chemokine activity in the subject.

Provided herein is a composition that comprises a) about 0.1% or more w/w of a secretome and b) a pharmaceutically acceptable excipient, wherein the secretome comprises one or more of SPP1, DKK1, SERPINE1, FLT1, FSTL3, MATN3, PAPPA, GDF15, HGF, IGFBP3, or any combination thereof. In one instance, the secretome comprises one, two, three, four, five, six, seven, eight, or nine of SPP1, DKK1, SERPINE1, FLT1, FSTL3, MATN3, PAPPA, GDF15, HGF, and IGFBP3. In another instance, the secretome comprises SPP1, DKK1, SERPINE1, FLT1, FSTL3, MATN3, PAPPA, GDF15, HGF, and IGFBP3. Any of such compositions are useful for inducing a biased expression in placenta. Provided herein is a method, comprising administering to a subject in need thereof any of such compositions, wherein the composition induces chemokine activity in the subject. Provided herein is a use of any of such compositions, for treating a subject in need thereof. Provided herein is a use of any of such compositions, for the manufacture of a medicament for treating a subject in need thereof.

Provided herein is a composition that comprises a) about 0.1% or more w/w of a secretome and b) a pharmaceutically acceptable excipient, wherein the secretome comprises one or more of FST, NID1, MET, TGFBI, FSTL1, NID2, CRIM1, PDGFB, or any combination thereof. In one instance, the secretome comprises one, two, three, four, five, six, or seven of FST, NID1, MET, TGFBI, FSTL1, NID2, CRIM1, and PDGFB. In another instance, the secretome comprises FST, NID1, MET, TGFBI, FSTL1, NID2, CRIM1, and PDGFB. Any of such compositions are useful for inducing a broad expression in placenta. Provided herein is a method, comprising administering to a subject in need thereof any of such compositions. Provided herein is a use of any of such compositions, for treating a subject in need thereof. Provided herein is a use of any of such compositions, for the manufacture of a medicament for treating a subject in need thereof.

Provided herein is a composition that comprises a) about 0.1% or more w/w of a secretome and b) a pharmaceutically acceptable excipient, wherein the secretome comprises one or more of CXCL12, LGALS1, ADAMTSL1, or any combination thereof. In one instance, the secretome comprises one or two of CXCL12, LGALS1, and ADAMTSL1. In another instance, the secretome comprises CXCL12, LGALS1, and ADAMTSL1. Any of such compositions are useful for inducing a broad endometrium expression. Provided herein is a method, comprising administering to a subject in need thereof any of such compositions. Provided herein is a use of any of such compositions, for treating a subject in need thereof. Provided herein is a use of any of such compositions, for the manufacture of a medicament for treating a subject in need thereof.

Provided herein is a composition that comprises a) about 0.1% or more w/w of a secretome and b) a pharmaceutically acceptable excipient, wherein the secretome comprises one or more of FAP, IGFBP3, or a combination thereof. In one instance, the secretome comprises FAP or IGFBP3. In another instance, the secretome comprises FAP and IGFBP3. Any of such compositions are useful for inducing a biased endometrium expression. Provided herein is a method, comprising administering to a subject in need thereof any of such compositions. Provided herein is a use of any of such compositions, for treating a subject in need thereof. Provided herein is a use of any of such compositions, for the manufacture of a medicament for treating a subject in need thereof.

Provided herein is a composition that comprises a) about 0.1% or more w/w of a secretome and b) a pharmaceutically acceptable excipient, wherein the secretome comprises one or more of CCL13, CCL20, CCL25, CCL26, CCL28, CCL4, CCL5, CCL7, CCL8, CXCL1, CXCL11, CXCL12, CXCL14, CXCL5, PF4, or a combination thereof. In one instance, the secretome comprises one, two, three, four, five, six, seven, eight, nine, ten, eleven, twelve, thirteen, or fourteen of CCL13, CCL20, CCL25, CCL26, CCL28, CCL4, CCL5, CCL7, CCL8, CXCL1, CXCL11, CXCL12, CXCL14, CXCL5, and PF4. In another instance, the secretome comprises CCL13, CCL20, CCL25, CCL26, CCL28, CCL4, CCL5, CCL7, CCL8, CXCL1, CXCL11, CXCL12, CXCL14, CXCL5, and PF4. Any of such compositions are useful for inducing chemokine activity. Provided herein is a method, comprising administering to a subject in need thereof any of such compositions. Provided herein is a use of any of such compositions, for treating a subject in need thereof that has a disease or condition that is treatable with chemokines. Provided herein is a use of any of such compositions, for the manufacture of a medicament for treating a subject in need thereof that has a disease or condition that is treatable with chemokines.

Provided herein is a composition that comprises a) about 0.1% or more w/w of a secretome and b) a pharmaceutically acceptable excipient, wherein the secretome comprises one or more of CSF1, GDF15, IFNL1, IFNL2, IL21, IL6, MIF, NAMPT, SPP1, TGFB1, TIMP1, or a combination thereof. In one instance, the secretome comprises one, two, three, four, five, six, seven, eight, nine, or ten of CSF1, GDF15, IFNL1, IFNL2, IL21, IL6, MIF, NAMPT, SPP1, TGFB1, and TIMP1. In another instance, the secretome comprises CSF1, GDF15, IFNL1, IFNL2, IL21, IL6, MIF, NAMPT, SPP1, TGFB1, and TIMP1. Any of such compositions are useful for inducing cytokine activity. Provided herein is a method, comprising administering to a subject in need thereof any of such compositions. Provided herein is a use of any of such compositions, for treating a subject in need thereof that has a disease or condition that is treatable with cytokines. Provided herein is a use of any of such compositions, for the manufacture of a medicament for treating a subject in need thereof that has a disease or condition that is treatable with cytokines.

Provided herein is a composition that comprises a) about 0.1% or more w/w of a secretome and b) a pharmaceutically acceptable excipient, wherein the secretome comprises one or more of CSF1, CXCL12, DKK1, GDF15, HGF, IL6, PDGFB, TGFB1, TIMP1, or a combination thereof. In one instance, the secretome comprises one, two, three, four, five, six, seven, or eight of CSF1, CXCL12, DKK1, GDF15, HGF, IL6, PDGFB, TGFB1, and TIMP1. In another instance, the secretome comprises CSF1, CXCL12, DKK1, GDF15, HGF, IL6, PDGFB, TGFB1, and TIMP1. Any of such compositions are useful for inducing growth. Provided herein is a method, comprising administering to a subject in need thereof any of such compositions. Provided herein is a use of any of such compositions, for treating a subject in need thereof that has a disease or condition that is treatable with growth factors. Provided herein is a use of any of such compositions, for the manufacture of a medicament for treating a subject in need thereof that has a disease or condition that is treatable with growth factors.

Provided herein is a composition that comprises a) about 0.1% or more w/w of a secretome and b) a pharmaceutically acceptable excipient, wherein the secretome comprises one or more of ANG, CSTB, NAP1L4, TLR3, or a combination thereof. In one instance, the secretome comprises one, two, or three of ANG, CSTB, NAP1L4, and TLR3. In another instance, the secretome comprises ANG, CSTB, NAP1L4, and TLR3. Any of such compositions are useful for inducing RNA binding activity. Provided herein is a method of inducing RNA binding activity in a subject in need thereof, comprising administering to the subject any of such compositions. Provided herein is a use of any of such compositions, for inducing RNA binding activity in a subject in need thereof. Provided herein is a use of any of such compositions, for the manufacture of a medicament for inducing RNA binding activity in a subject in need thereof.

Provided herein is a composition that comprises a) about 0.1% or more w/w of a secretome and b) a pharmaceutically acceptable excipient, wherein the secretome comprises one or more of ADAMTSL1, DCN, FURIN, LUM, MATN3, MMP1, NID1, NID2, PDGFB, POSTN, PTX3, SERPINE, SPP1, TGFβI, THBS1, TIMP1, TIMP2, CCN1, LUM, MATN3, NID1, NID2, POSTN, or a combination thereof. In one instance, the secretome comprises one, two, three, four, five, six, seven, eight, nine, ten, eleven, twelve, thirteen, fourteen, fifteen, sixteen, seventeen, eighteen, nineteen, twenty, or twenty-one of ADAMTSL1, DCN, FURIN, LUM, MATN3, MMP1, NID1, NID2, PDGFB, POSTN, PTX3, SERPINE, SPP1, TGFβI, THBS1, TIMP1, TIMP2, CCN1, LUM, MATN3, NID1, NID2, and POSTN. In another instance, the secretome comprises ADAMTSL1, DCN, FURIN, LUM, MATN3, MMP1, NID1, NID2, PDGFB, POSTN, PTX3, SERPINE, SPP1, TGFβI, THBS1, TIMP1, TIMP2, CCN1, LUM, MATN3, NID1, NID2, and POSTN. Any of such compositions are useful for inducing extracellular matrix organization. Provided herein is a method of inducing extracellular matrix organization in a subject in need thereof, comprising administering to a subject in need thereof any of such compositions. Provided herein is a use of any of such compositions, for inducing extracellular matrix organization in a subject in need thereof. Provided herein is a use of any of such compositions, for the manufacture of a medicament for inducing extracellular matrix organization in a subject in need thereof.

Provided herein is a composition that comprises a) about 0.1% or more w/w of a secretome and b) a pharmaceutically acceptable excipient, wherein the secretome comprises one or more of ANG, B2M, BCL10, CCL13, CCL20, CCL25, CCL28, CCL4, CD99, CLU, CSF1, CXCL1, CXCL1, CXCL11, CXCL12, CXCL14, CXCL5, F11R, FAS, IFNL1, IFNL2, IL21, IL6, IL7R, LGALS3, MIF, OSCAR, PF4, PTX3, SERPINE1, SIGLEC9, THBS1, TLR3, TNFRSF21, or a combination thereof. In one instance, the secretome comprises one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, or 33 of ANG, B2M, BCL10, CCL13, CCL20, CCL25, CCL28, CCL4, CD99, CLU, CSF1, CXCL1, CXCL1, CXCL11, CXCL12, CXCL14, CXCL5, F11R, FAS, IFNL1, IFNL2, IL21, IL6, IL7R, LGALS3, MIF, OSCAR, PF4, PTX3, SERPINE1, SIGLEC9, THBS1, TLR3, and TNFRSF21. In another instance, the secretome comprises ANG, B2M, BCL10, CCL13, CCL20, CCL25, CCL28, CCL4, CD99, CLU, CSF1, CXCL1, CXCL1, CXCL11, CXCL12, CXCL14, CXCL5, F11R, FAS, IFNL1, IFNL2, IL21, IL6, IL7R, LGALS3, MIF, OSCAR, PF4, PTX3, SERPINE1, SIGLEC9, THBS1, TLR3, and TNFRSF21. Any of such compositions are useful for inducing an immune response. Provided herein is a method of inducing an immune response in a subject in need thereof, comprising administering to a subject in need thereof any of such compositions. Provided herein is a use of any of such compositions, for inducing an immune response in a subject in need thereof. Provided herein is a use of any of such compositions, for the manufacture of a medicament for inducing an immune response in a subject in need thereof.

Provided herein is a composition that comprises a) about 0.1% or more w/w of a secretome and b) a pharmaceutically acceptable excipient, wherein the secretome comprises one or more of CCL13, CCL13, CCL20, CCL25, CCL4, CCL5, CCL7, CCL8, CSF1, CXCL1, CXCL12, F11R, IGFBP4, IL6, MIF, NUP85, PF4, PTX3, SEMA7A, SPP1, THBS1, TNFRSF1A, or a combination thereof. In one instance, the secretome comprises one, two, three, four, five, six, seven, eight, nine, ten, eleven, twelve, thirteen, fourteen, fifteen, sixteen, seventeen, eighteen, nineteen, twenty, or twenty-one, of CCL13, CCL13, CCL20, CCL25, CCL4, CCL5, CCL7, CCL8, CSF1, CXCL1, CXCL12, F11R, IGFBP4, IL6, MIF, NUP85, PF4, PTX3, SEMA7A, SPP1, THBS1, and TNFRSF1A. In another instance, the secretome comprises CCL13, CCL13, CCL20, CCL25, CCL4, CCL5, CCL7, CCL8, CSF1, CXCL1, CXCL12, F11R, IGFBP4, IL6, MIF, NUP85, PF4, PTX3, SEMA7A, SPP1, THBS1, and TNFRSF1A. Any of such compositions are for use in treating inflammation. Provided herein is a method of treating inflammation in a subject in need thereof, comprising administering to a subject any of such compositions. Provided herein is a use of any of such compositions, for treating inflammation in a subject in need thereof. Provided herein is a use of any of such compositions, for the manufacture of a medicament for treating inflammation in a subject in need thereof.

Provided herein is a composition that comprises a) about 0.1% or more w/w of a secretome and b) a pharmaceutically acceptable excipient, wherein the secretome comprises one or more of ANG, CCL13, CCL20, CCL25, CCL26, CCL8, CLU, CXCL1, CXCL11, CXCL12, CXCL14, CXCL5, LGALS3, PF4, ANG, B2M, CCL20, KLK3, TLR3, TNFRSF1A, CCL4, IFNL1, IFNL2, IL21, IL6, TLR3, or a combination thereof. In one instance, the secretome comprises one, two, three, four, five, six, seven, eight, nine, ten, eleven, twelve, thirteen, fourteen, fifteen, sixteen, seventeen, eighteen, nineteen, twenty, 21, 22, 23, 24, or 25 of ANG, CCL13, CCL20, CCL25, CCL26, CCL8, CLU, CXCL1, CXCL11, CXCL12, CXCL14, CXCL5, LGALS3, PF4, ANG, B2M, CCL20, KLK3, TLR3, TNFRSF1A, CCL4, IFNL1, IFNL2, IL21, IL6, and TLR3. In another instance, the secretome comprises ANG, CCL13, CCL20, CCL25, CCL26, CCL8, CLU, CXCL1, CXCL11, CXCL12, CXCL14, CXCL5, LGALS3, PF4, ANG, B2M, CCL20, KLK3, TLR3, TNFRSF1A, CCL4, IFNL1, IFNL2, IL21, IL6, and TLR3. Any of such compositions can exhibit anti-microbial activity. In one instance, secretome comprises one or more of ANG, B2M, CCL20, KLK3, TLR3, TNFRSF1A, or any combination thereof. In another instance, the secretome comprises ANG, B2M, CCL20, KLK3, TLR3, and TNFRSF1A. Any of such compositions can exhibit anti-bacterial activity. In one instance, the secretome comprises one or more of CCL4, IFNL1, IFNL2, IL21, IL6, TLR3, or any combination thereof. In another instance, the secretome comprises CCL4, IFNL1, IFNL2, IL21, IL6, and TLR3. Any of such compositions can exhibit anti-viral activity. Any of such compositions can be for use in treating a microbial infection. Provided herein is a method of treating a microbial infection in a subject in need thereof, comprising administering to a subject in need thereof any of such compositions. Provided herein is a use of any of such compositions, for treating a microbial infection in a subject in need thereof. Provided herein is a use of any of such compositions, for the manufacture of a medicament for treating a microbial infection in a subject in need thereof.

Provided herein is a composition that comprises a) about 0.1% or more w/w of a secretome and b) a pharmaceutically acceptable excipient, wherein the secretome comprises one or more of DCN, POSTN, SDC4, GRN, PAPPA, TIMP1, or a combination thereof. In one instance, the secretome comprises one, two, three, four, or five of DCN, POSTN, SDC4, GRN, PAPPA, and TIMP1. In another instance, the secretome comprises DCN, POSTN, SDC4, GRN, PAPPA, and TIMP1. Such secretomes are useful for wound healing. Provided herein is a method of wound healing in a subject in need thereof, comprising administering to a subject in need thereof any of such compositions. Provided herein is a use of any of such compositions, for wound healing in a subject in need thereof. Provided herein is a use of any of such compositions, for the manufacture of a medicament for wound healing in a subject in need thereof.

Provided herein is a composition that comprises a) about 0.1% or more w/w of a secretome and b) a pharmaceutically acceptable excipient, wherein the secretome comprises one or more of ANGPT1, FLT1, MET, CST3, DKK3, RBP4, BSG, or a combination thereof. In one instance, the secretome comprises one, two, three, four, five, or six of ANGPT1, FLT1, MET, CST3, DKK3, RBP4, and BSG. In another instance, the secretome comprises ANGPT1, FLT1, MET, CST3, DKK3, RBP4, and BSG. Such secretomes are useful for inducing embryonic development. Provided herein is a method of inducing embryonic development in a subject in need thereof, comprising administering to a subject in need thereof any of such compositions. Provided herein is a use of any of such compositions, for inducing embryonic development in a subject in need thereof. Provided herein is a use of any of such compositions, for the manufacture of a medicament for inducing embryonic development in a subject in need thereof.

Provided herein is a composition that comprises a) about 0.1% or more w/w of a secretome and b) a pharmaceutically acceptable excipient, wherein the secretome comprises one or more of ANG, DCN, BIRC2, PDGFB, or a combination thereof. In one instance, the secretome comprises one, two, three, four, five, six, seven, eight, nine, ten, eleven, twelve, thirteen, or fourteen of ANG, DCN, BIRC2, and PDGFB. In another instance, the secretome comprises ANG, DCN, BIRC2, and PDGFB. Such secretomes are useful for inducing placental development. Provided herein is a method of inducing placental development in a subject in need thereof, comprising administering to a subject in need thereof any of such compositions. Provided herein is a use of any of such compositions, for inducing placental development in a subject in need thereof. Provided herein is a use of any of such compositions, for the manufacture of a medicament for inducing placental development in a subject in need thereof.

Provided herein is a composition that comprises a) about 0.1% or more w/w of a secretome and b) a pharmaceutically acceptable excipient, wherein the secretome comprises one or more of TIMP4, CRIM1, UNC5C, TIMP2, or a combination thereof. In one instance, the secretome comprises one, two, or three of TIMP4, CRIM1, UNC5C, and TIMP2. In another instance, the secretome comprises TIMP4, CRIM1, UNC5C, and TIMP2. Such secretomes are useful for inducing central nervous system (CNS) development. Provided herein is a method of inducing CNS development in a subject in need thereof, comprising administering to a subject in need thereof any of such compositions. Provided herein is a use of any of such compositions, for inducing CNS development in a subject in need thereof. Provided herein is a use of any of such compositions, for the manufacture of a medicament for inducing CNS development in a subject in need thereof.

Provided herein is a composition that comprises a) about 0.1% or more w/w of a secretome and b) a pharmaceutically acceptable excipient, wherein the secretome comprises one or more of DKK1, FST, TGFB1, FLT1, DKK3, CCN1, or a combination thereof. In one instance, the secretome comprises one, two, three, four, or five of DKK1, FST, TGFB1, FLT1, DKK3, and CCN1. In another instance, the secretome comprises DKK1, FST, TGFB1, FLT1, DKK3, and CCN1. Such secretomes are useful for inducing morphogenesis. Provided herein is a method of inducing morphogenesis in a subject in need thereof, comprising administering to a subject in need thereof any of such compositions. Provided herein is a use of any of such compositions, for inducing morphogenesis in a subject in need thereof. Provided herein is a use of any of such compositions, for the manufacture of a medicament for inducing morphogenesis in a subject in need thereof.

Provided herein is a composition that comprises a) about 0.1% or more w/w of a secretome and b) a pharmaceutically acceptable excipient, wherein the secretome comprises one or more of NAP1L4, SPP1, ANGPT1, FST, MET, CTSB, FSTL1, LGALS1, TPP1, OSCAR, CCN1, IGFBP3, TGFB1, B2M, IL7R, CES1, CSF1, or any combination thereof. In one instance, the secretome comprises one, two, three, four, five, six, seven, eight, nine, ten, eleven, twelve, thirteen, fourteen, fifteen, or sixteen of NAP1L4, SPP1, ANGPT1, FST, MET, CTSB, FSTL1, LGALS1, TPP1, OSCAR, CCN1, IGFBP3, TGFB1, B2M, IL7R, CES1, and CSF1. In another instance, the secretome comprises NAP1L4, SPP1, ANGPT1, FST, MET, CTSB, FSTL1, LGALS1, TPP1, OSCAR, CCN1, IGFBP3, TGFB1, B2M, IL7R, CES1, and CSF1. Such secretomes are useful for inducing differentiation of a stem cell. In one instance, the secretome comprises one or more of SPP1, OSCAR, CCN1, IGFBP3, or any combination thereof. In another instance, the secretome comprises SPP1, OSCAR, CCN1, and IGFBP3. Such secretomes are useful for inducing differentiation of a stem cell into an osteoblast. The In one instance, the secretome comprises CSF1. Such secretomes are useful for inducing differentiation of a stem cell into an osteoclast or a macrophage. In one instance, the secretome comprises B2M, IL7R, or a combination thereof. In another instance, the secretome comprises B2M and IL7R. Such secretomes are useful for inducing differentiation of a stem cell into a T cell. In one instance, the secretome comprises one or more of CTSB, TPP1, CES1, or any combination thereof. In another instance, the secretome comprises CTSB, TPP1, and CES1. Such secretomes are useful for inducing differentiation of a stem cell into an epithelial cell.

In one instance, the secretome comprises MET. Such secretomes are useful for inducing differentiation of a stem cell into a neuron. In one instance, the secretome comprises CCN1. Such secretomes are useful for inducing differentiation of a stem cell into a chondroblast. In one instance, the secretome comprises TGFβ1. Such secretomes are useful for inducing differentiation of a stem cell into a chondrocyte. In one instance, the secretome comprises LGALS1. Such secretomes are useful for inducing differentiation of a stem cell into a myoblast. Provided herein is a method of inducing cellular differentiation in vitro, ex vivo, or in vivo in a subject in need thereof, comprising administering any of such compositions. Provided herein is a u se of any of such compositions, for inducing cellular differentiation in vitro, ex vivo, or in vivo in a subject in need thereof. Provided herein is a use of any of such compositions, for the manufacture of a medicament for inducing cellular differentiation in vitro, ex vivo, or in vivo in a subject in need thereof.

Provided herein is a composition that comprises a) about 0.1% or more w/w of a secretome and b) a pharmaceutically acceptable excipient, wherein the secretome comprises one or more of SIGLEC9, POSTN, TGFβI, or a combination thereof. In one instance, the secretome comprises one or two of SIGLEC9, POSTN, and TGFβI. In another instance, the secretome comprises SIGLEC9, POSTN, and TGFβI. Provided herein is a method of inducing or promoting cell adhesion in a subject in need thereof, comprising administering to a subject in need thereof any of such compositions. Provided herein is a use of any of such compositions, for inducing or promoting cell adhesion in a subject in need thereof, or for the manufacture of a medicament for inducing or promoting cell adhesion in a subject in need thereof.

Provided herein is a composition that comprises a) about 0.1% or more w/w of a secretome and b) a pharmaceutically acceptable excipient, wherein the secretome comprises one or more of OSCAR, B2M, or a combination thereof. In one instance, the secretome comprises OSCAR or B2M. In another instance, the secretome comprises OSCAR and B2M. Provided herein is a method of improving or enhancing immunity in a subject in need thereof, comprising administering to a subject in need thereof any of such compositions. Provided herein is a use of any of such compositions, for improving or enhancing in a subject in need thereof, or for the manufacture of a medicament for improving or enhancing in a subject in need thereof.

Provided herein is a composition that comprises a) about 0.1% or more w/w of a secretome and b) a pharmaceutically acceptable excipient, wherein the secretome comprises one or more of LGALS3, LGALS1, or a combination thereof. In one instance, the secretome comprises LGALS3 or LGALS1. In another instance, the secretome comprises LGALS3 and LGALS1. Provided herein is a method of inducing or enhancing extracellular matrix in a subject in need thereof, comprising administering to a subject in need thereof any of such compositions. Provided herein is a use of any of such compositions, for inducing or enhancing extracellular matrix in a subject in need thereof, or for the manufacture of a medicament for inducing or enhancing extracellular matrix in a subject in need thereof.

Provided herein is a composition that comprises a) about 0.1% or more w/w of a secretome and b) a pharmaceutically acceptable excipient, wherein the secretome comprises one or more of XCL1, TGFβ1, CCL5, CCL20, CCL28, CCL4, CXCL5, ANGPTL4, PDGFB, CCL13, CCL8, ANGPTI, CCL25, CXCL11, CCL7, PF4, GDF15, CCL26, SEMA7A, SPP1, CXCL1, or a combination thereof. In one instance, the secretome comprises 1, 2, 3, 4, 5, 6, 7, 8, 9, 10, 11, 12, 13, 14, 15, 16, 17, 18, 19, or 20 of XCL1, TGFβ1, CCL5, CCL20, CCL28, CCL4, CXCL5, ANGPTL4, PDGFB, CCL13, CCL8, ANGPTI, CCL25, CXCL11, CCL7, PF4, GDF15, CCL26, SEMA7A, SPP1, and CXCL1. In another instance, the secretome comprises XCL1, TGFβ1, CCL5, CCL20, CCL28, CCL4, CXCL5, ANGPTL4, PDGFB, CCL13, CCL8, ANGPTI, CCL25, CXCL11, CCL7, PF4, GDF15, CCL26, SEMA7A, SPP1, and CXCL1. In another instance, the secretome comprises cytokine activity, and the secretome comprises XCL1, CCL5, CCL20, CCL28, CCL4, CXCL5, CCL13, CCL8, CCL25, CXCL11, CCL7, PF4, CCL26, SPP1, CXCL1, or a combination thereof. In another instance, the secretome comprises cytokine activity, and the secretome comprises XCL1, CCL5, CCL20, CCL28, CCL4, CXCL5, CCL13, CCL8, CCL25, CXCL11, CCL7, PF4, CCL26, SPP1, and CXCL1. In another instance, the secretome comprises growth factor activity, and the secretome comprises TGFβI, PDGFB, GDF15, or a combination thereof. In another instance, the secretome comprises growth factor activity, and the secretome comprises TGFβI, PDGFB, and GDF15. In another instance, the secretome comprises signaling molecules, and the secretome comprises SEMA7A. Provided herein is a method of inducing or enhancing intracellular signaling in a subject in need thereof, comprising administering to a subject in need thereof any of such compositions. Provided herein is a use of any of such compositions, for inducing or enhancing intracellular signaling in a subject in need thereof, or for the manufacture of a medicament for inducing or enhancing intracellular signaling in a subject in need thereof.

Provided herein is a composition that comprises a) about 0.1% or more w/w of a secretome and b) a pharmaceutically acceptable excipient, wherein the secretome comprises one or more of CES1, FAS, MIF, NAMPT, SPP1, or a combination thereof. In one instance, the secretome comprises one, two, three, or four of CES1, FAS, MIF, NAMPT, and SPP1. In another instance, the secretome comprises CES1, FAS, MIF, NAMPT, and SPP1. Provided herein is a method of inducing or enhancing the activity of metabolite interconversion enzymes in a subject in need thereof, comprising administering to a subject in need thereof any of such compositions. Provided herein is a use of any of such compositions, for inducing or enhancing the activity of metabolite interconversion enzymes in a subject in need thereof, or for the manufacture of a medicament for inducing or enhancing the activity of metabolite interconversion enzymes in a subject in need thereof.

Provided herein is a composition that comprises a) about 0.1% or more w/w of a secretome and b) a pharmaceutically acceptable excipient, wherein the secretome comprises one or more of FAP, LGMN, HGF, CTSB, TPP1, KLK3, FURIN, MMP1, or a combination thereof. In one instance, the secretome comprises 1, 2, 3, 4, 5, 6, or 7 of FAP, LGMN, HGF, CTSB, TPP1, KLK3, FURIN, and MMP1. In another instance, the secretome comprises FAP, LGMN, HGF, CTSB, TPP1, KLK3, FURIN, MMP1. Provided herein is a method of inducing or enhancing the activity of protein-modifying enzymes/proteases in a subject in need thereof, comprising administering to a subject in need thereof any of such compositions. Provided herein is a use of any of such compositions, for inducing or enhancing the activity of protein-modifying enzymes/proteases in a subject in need thereof, or for the manufacture of a medicament for inducing or enhancing the activity of protein-modifying enzymes/proteases in a subject in need thereof.

Provided herein is a composition that comprises a) about 0.1% or more w/w of a secretome and b) a pharmaceutically acceptable excipient, wherein the secretome comprises one or more of IGFBP3, TIMP4, FSTL1, BIRC2, FST, SERPINE1, TIMP1, IGFBP2, FSTL3, IGFBP4, TIMP2, or a combination thereof. In one instance, the secretome comprises 1, 2, 3, 4, 5, 6, 7, 8, 9, or 10 of IGFBP3, TIMP4, FSTL1, BIRC2, FST, SERPINE1, TIMP1, IGFBP2, FSTL3, IGFBP4, and TIMP2. In another instance, the secretome comprises IGFBP3, TIMP4, FSTL1, BIRC2, FST, SERPINE1, TIMP1, IGFBP2, FSTL3, IGFBP4, and TIMP2. Provided herein is a method of inducing or enhancing the activity of protein-binding modulators/protease inhibitors in a subject in need thereof, comprising administering to a subject in need thereof any of such compositions. Provided herein is a use of any of such compositions, for inducing or enhancing the activity of protein-binding modulators/protease inhibitors in a subject in need thereof, or for the manufacture of a medicament for inducing or enhancing the activity of protein-binding modulators/protease inhibitors in a subject in need thereof.

Provided herein is a composition that comprises a) about 0.1% or more w/w of a secretome and b) a pharmaceutically acceptable excipient, wherein the secretome comprises BSG. Provided herein is a method of inducing or enhancing the activity of scaffold/adaptor proteins in a subject in need thereof, comprising administering to a subject in need thereof any of such compositions. Provided herein is a use of any of such compositions, for inducing or enhancing the activity of scaffold/adaptor proteins in a subject in need thereof, or for the manufacture of a medicament for inducing or enhancing the activity of scaffold/adaptor proteins in a subject in need thereof.

Provided herein is a composition that comprises a) about 0.1% or more w/w of a secretome and b) a pharmaceutically acceptable excipient, wherein the secretome comprises NUP85. Provided herein is a method of inducing or enhancing the activity of structural proteins in a subject in need thereof, comprising administering to a subject in need thereof any of such compositions. Provided herein is a use of any of such compositions, for inducing or enhancing the activity of structural proteins in a subject in need thereof, or for the manufacture of a medicament for inducing or enhancing the activity of structural proteins in a subject in need thereof.

Provided herein is a composition that comprises a) about 0.1% or more w/w of a secretome and b) a pharmaceutically acceptable excipient, wherein the secretome comprises one or more of ALB, TPP1, LDLR, RBP4, TF, or a combination thereof. In one instance, the secretome comprises one, two, three, or four of ALB, TPP1, LDLR, RBP4, and TF. In another instance, the secretome comprises LDLR. In another instance, the secretome comprises ALB, TPP1, LDLR, RBP4, and TF. Provided herein is a method of inducing or enhancing the activity of transfer/carrier proteins in a subject in need thereof, comprising administering to a subject in need thereof any of such compositions. Provided herein is a use of any of such compositions for inducing or enhancing the activity of transfer/carrier proteins in a subject in need thereof, or for the manufacture of a medicament for inducing or enhancing the activity of transfer/carrier proteins in a subject in need thereof.

Provided herein is a composition that comprises a) about 0.1% or more w/w of a secretome and b) a pharmaceutically acceptable excipient, wherein the secretome comprises one or more of UNC5C, TLR3, PLAUR, GP1BA, SDC4, THBD, IL7R, TF, or a combination thereof. In one instance, the secretome comprises one, two, three, four, five, six, or seven of LTNCSC, TLR3, PLAUR, GP1BA, SDC4, THBD, IL7R, and TF. In another instance, the secretome comprises UNC5C, TLR3, PLAUR, GP1BA, SDC4, THBD, IL7R, and TF. Provided herein is a method of inducing or enhancing the activity of transmembrane signal receptors in a subject in need thereof, comprising administering to a subject in need thereof any of such compositions. Provided herein is a use of any of such compositions for inducing or enhancing the activity of transmembrane signal receptors in a subject in need thereof, or for the manufacture of a medicament for inducing or enhancing the activity of transmembrane signal receptors in a subject in need thereof.

Provided herein is a composition that comprises a) about 0.1% or more w/w of a secretome and b) a pharmaceutically acceptable excipient, wherein the secretome comprises one or more of SIGLEC9, CD99, TNFRSF21, GP1BA, BSG, POSTN, TGFβI, or a combination thereof. In one instance, the secretome comprises one, two, three, four, five, or six of SIGLEC9, CD99, TNFRSF21, GP1BA, BSG, POSTN, and TGFβI. In another instance, the secretome comprises SIGLEC9, CD99, TNFRSF21, GP1BA, BSG, POSTN, and TGFBI. Provided herein is a method of inducing cell adhesion in a subject in need thereof, comprising administering to a subject in need thereof any of such compositions. Provided herein is a use of any of such compositions for inducing cell adhesion in a subject in need thereof, or for the manufacture of a medicament for inducing cell adhesion in a subject in need thereof.

Provided herein is a composition that comprises a) about 0.1% or more w/w of a secretome and b) a pharmaceutically acceptable excipient, wherein the secretome comprises one or more of XCL1, IGFBP3, TGFβ1, CCL5, TIMP4, CCL20, FSTL1, CCL4, BIRC2, BCL10, CD99, LGALS3, CXCL5, TNFRSF21, FST, SERPINE1, GP1BA, PDGFB, F11R, CCL13, TIMP1, NID1, CCL8, NUP85, IGFBP6, THBS1, CCL25, IGFBP2, FSTL3, IGFBP4, KLK3, CXCL11, GPC1, CCL7, DKK3, PF4, GDF15, TF, CCL26, TIMP2, SEMA7A, FURIN, DKK1, INFRSF10C, CXCL 1, or a combination thereof. In one instance, the secretome comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 431, 42, 43, or 44 of XCL1, IGFBP3, TGFβ1, CCL5, TIMP4, CCL20, FSTL1, CCL4, BIRC2, BCL10, CD99, LGALS3, CXCL5, TNFRSF21, FST, SERPINE1, GP1BA, PDGFB, F11R, CCL13, TIMP1, NID1, CCL8, NUP85, IGFBP6, THBS1, CCL25, IGFBP2, FSTL3, IGFBP4, KLK3, CXCL11, GPC1, CCL7, DKK3, PF4, GDF15, TF, CCL26, TIMP2, SEMA7A, FURIN, DKK1, INFRSF10C, and CXCL1. In another instance, the secretome comprises XCL1, IGFBP3, TGFβ1, CCL5, TIMP4, CCL20, FSTL1, CCL4, BIRC2, BCL10, CD99, LGALS3, CXCL5, TNFRSF21, FST, SERPINE1, GP1BA, PDGFB, F11R, CCL13, TIMP1, NID1, CCL8, NUP85, IGFBP6, THBS1, CCL25, IGFBP2, FSTL3, IGFBP4, KLK3, CXCL11, GPC1, CCL7, DKK3, PF4, GDF15, TF, CCL26, TIMP2, SEMA7A, FURIN, DKK1, INFRSF10C, and CXCL1. Provided herein is a method of inducing biological regulation in a subject in need thereof, comprising administering to a subject in need thereof any of such compositions. “Biological regulation” as used herein refers to communication between cells via their secreted substances. e.g. secretome of hTSCs and its effects in regulating systemic inflammation or wound healing. Provided herein is a use of any of such compositions, for inducing biological regulation in a subject in need thereof, or for the manufacture of a medicament for inducing biological regulation in a subject in need thereof.

Provided herein is a composition that comprises a) about 0.1% or more w/w of a secretome and b) a pharmaceutically acceptable excipient, wherein the secretome comprises one or more of TGFβ1, TNFRSF21, PDGFB, or a combination thereof. In one instance, the secretome comprises one or two of TGFB1, TNFRSF21, and PDGFB. In another instance, the secretome comprises TGFβ1, TNFRSF21, and PDGFB. Provided herein is a method of inducing cell proliferation in a subject in need thereof, comprising administering to a subject in need thereof any of such compositions. Provided herein is a use of any of such compositions for inducing cell proliferation in a subject in need thereof, or for the manufacture of a medicament for inducing cell proliferation in a subject in need thereof.

Provided herein is a composition that comprises a) about 0.1% or more w/w of a secretome and b) a pharmaceutically acceptable excipient, wherein the secretome comprises one or more of UNC5C, BIRC2, LGALS3, BSG, POSTN, TGFβI, SEMA7A, MMP1, or a combination thereof. In one instance, the secretome comprises one, two, three, four, five, six, or seven of UNC5C, BIRC2, LGALS3, BSG, POSTN, TGFβI, SEMA7A, and MMP1. In another instance, the secretome comprises UNC5C, BIRC2, LGALS3, BSG, POSTN, TGFβI, SEMA7A, and MMP1. Provided herein is a method of inducing cellular organization or biogenesis in a subject in need thereof, comprising administering to a subject in need thereof any of such compositions. Provided herein is a use of any of such compositions for inducing cellular organization or biogenesis in a subject in need thereof, or for the manufacture of a medicament for inducing cellular organization or biogenesis in a subject in need thereof.

Provided herein is a composition that comprises a) about 0.1% or more w/w of a secretome and b) a pharmaceutically acceptable excipient, wherein the secretome comprises: a. TNFRSF21, GP1BA, or a combination thereof, wherein the secretome induces cell activation; b. XCL1, IGFBP3, TGFB1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof, wherein the secretome induces cellular communication; c. BIRC2, wherein the secretome induces a cell cycle process or a microtubule-based process; d. BIRC2, BCL10, LGALS3, TNFRSF21, INFRSF10C, or a combination thereof, wherein the secretome induces cell death; e. SEMA7A, wherein the secretome induces cell growth; f. UNC5C, BIRC2, LGALS3, BSG, POSTN, TGFβI, SEMA7A, MMP1, or a combination thereof, wherein the secretome induces Cellular component organization; g. UNC5C, FSTL1, FST, MET, F11R, BSG, FLT1, FSTL3, SEMA7A, or a combination thereof, wherein the secretome induces a cellular developmental process; h. UNC5C, FSTL1, FST, MET, F11R, BSG, FLT1, FSTL3, SEMA7A, or a combination thereof, wherein the secretome induces cell differentiation; i. UNC5C, BSG, SEMA7A, or a combination thereof, wherein the secretome induces morphogenesis; j. XCL1, TGFβ1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, CCL26, TIMP2, SEMA7A, FURIN, or a combination thereof, wherein the secretome induces a cellular metabolic process; k. XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof, wherein the secretome induces a cellular response to stimulus; 1. TNFRSF21, wherein the secretome induces export from a cell; m. TNFRSF21, GP1BA, or a combination thereof, wherein the secretome induces cellular activation; or n. XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof, wherein the secretome induces cellular communication.

Provided herein is a composition that comprises a) about 0.1% or more w/w of a secretome and b) a pharmaceutically acceptable excipient, wherein the secretome comprises: a. XCL1, CCL5, CCL20, CCL4, CD99, LGALS3, CXCL5, MET, PDGFB, CCL13, SDC4, CCL8, CCL25, CXCL11, GPC1, CCL7, PF4, CCL26, SEMA7A, CXCL1, or a combination thereof, wherein the secretome induces cell motility; b. UNC5C, BSG, SEMA7A, or a combination thereof, wherein the secretome induces neuron projection guidance; c. TNFRSF21, wherein the secretome induces myelination; or d. XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof, wherein the secretome induces signal transduction.

Provided herein is a composition that comprises a) about 0.1% or more w/w of a secretome and b) a pharmaceutically acceptable excipient, wherein the secretome comprises: a. LTNCSC, FSTL1, PTX3, TNFRSF21, FST, MET, FUR, BSG, THBS1, FLT1, FSTL3, SEMA7A, or a combination thereof, wherein the secretome induces a developmental process; b. LTNCSC, FSTL1, PTX3, TNFRSF21, FST, MET, FUR, BSG, THBS1, FLT1, FSTL3, SEMA7A, or a combination thereof, wherein the secretome induces anatomical structure development; c. THBS1, wherein the secretome induces anatomical structure formation involved in morphogenesis; d. UNC5C, PTX3, BSG, THBS1, SEMA7A, or a combination thereof, wherein the secretome induces anatomical structure morphogenesis; e. UNC5C, FSTL1, FST, MET, F11R, BSG, FLT1, FSTL3, SEMA7A, or a combination thereof, wherein the secretome induces a cellular developmental process; or f. SEMA7A, or a combination thereof, wherein the secretome induces growth.

Provided herein is a composition that comprises a) about 0.1% or more w/w of a secretome and b) a pharmaceutically acceptable excipient, wherein the secretome comprises: a. IFNL1, SEMA7A, or a combination thereof, wherein the secretome induces an immune effector process; b. XCL1, CCL5, CCL20, CCL4, BCL10, CXCL5, TNFRSF21, PTX3, CCL13, IFNL1, CCL8, FLT1, CCL25, CXCL11, CCL7, PF4, CCL26, SEMA7A, CXCL1, or a combination thereof, wherein the secretome induces an immune response; c. FLT1, wherein the secretome induces immune system development; d. TNFRSF21, wherein the secretome induces leukocyte activation; or e. XCL1, CCL5, CCL20, CCL4, BCL10, CD99, LGALS3, CXCL5, CCL13, CCL8, CXCL11, CCL7, PF4, CCL26, CXCL1, or a combination thereof, wherein the secretome induces leukocyte migration.

Provided herein is a composition that comprises a) about 0.1% or more w/w of a secretome and b) a pharmaceutically acceptable excipient, wherein the secretome comprises: a. NUP85, GPC1, or a combination thereof, wherein the secretome induces cellular localization; b. ALB, LGALS3, TNFRSF21, MET, F11R, NUP85, or a combination thereof, wherein the secretome induces establishment of localization; c. XCL1, CCL5, CCL20, CCL4, CD99, LGALS3, CXCL5, MET, PDGFB, CCL13, SDC4, CCL8, CCL25, CXCL11, GPC1, CCL7, PF4, CCL26, SEMA7A, CXCL1, or a combination thereof, wherein the secretome induces localization of cell; d. TNFRSF21, NUP85, GPC1, or a combination thereof, wherein the secretome induces macromolecule localization; e. XCL1, CCL5, CCL20, CCL4, CD99, LGALS3, CXCL5, MET, PDGFB, CCL13, SDC4, CCL8, or a combination thereof, wherein the secretome induces cell motility; f. CCL25, CXCL11, GPC1, CCL7, PF4, CCL26, SEMA7A, CXCL1, or a combination thereof; or g. XCL1, UNC5C, CCL5, CCL20, CCL4, LGALS3, CXCL5 CCL13, BSG, CCL8, CCL25, CXCL11, CCL7, PF4, CCL26, SEMA7A, CXCL1, or a combination thereof, wherein the secretome induces taxis.

Provided herein is a composition that comprises a) about 0.1% or more w/w of a secretome and b) a pharmaceutically acceptable excipient, wherein the secretome comprises: a. TGFB1, FSTL1, BCL10, FST, NUP85, FSTL3, GDF15, or a combination thereof, wherein the secretome induces a biosynthetic process; b. TIMP4, LGMN, CED1, TIMP1, CTSB, TIMP2, MMP1, or a combination thereof, wherein the secretome induces a catabolic process; c. XCL1, TGFB1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, CCL26, TIMP2, SEMA7A, FURIN, or a combination thereof, wherein the secretome induces a cellular metabolic process; d. FURIN, wherein the secretome induces a hormone metabolic process; e. XCL1, TGFB1, CCL5, TIMP4, CCL20, FSTL1, CCL4, BIRC2, BCL10, LGMN, FST, SERPINE1, PDGFB, CCL13, TIMP1, CCL8, CTSB, NUP85, CCL25, FSTL3, CCL7, GDF15, or a combination thereof, wherein the secretome induces a nitrogen compound metabolic process; or CCL26, TIMP2, SEMA7A, FURIN, or a combination thereof.

Provided herein is a composition that comprises a) about 0.1% or more w/w of a secretome and b) a pharmaceutically acceptable excipient, wherein the secretome comprises XCL1, CCL5, CCL20, CCL4, CXCL5, PTX3, CCL13, IFNL1, CCL8, CCL25, CXCL11, CCL7, PF4, CCL26, CXCL1, or a combination thereof.

Provided herein is a composition that comprises a) about 0.1% or more w/w of a secretome and b) a pharmaceutically acceptable excipient, wherein the secretome comprises: a. GP1BA, wherein the secretome induces coagulation; b. TNFRSF21, SEMA7A, or a combination thereof, wherein the secretome induces cytokine production; c. f11R, wherein the secretome induces digestion; d. UNC5C, FSTL1, TNFRSF21, FST, MET, BSG, THBS1, FLT1, FSTL3, SEMA7A, or a combination thereof, wherein the secretome induces multicellular organism development; or e. TNFRSF21, FUR, KLK3, or a combination thereof, wherein the secretome induces a system process.

Provided herein is a composition that comprises a) about 0.1% or more w/w of a secretome and b) a pharmaceutically acceptable excipient, wherein the secretome comprises: a. XCL1, IGFBP3, TGFB1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof, wherein the secretome induces a cellular response to stimulus; or b. XCL1, CCL5, CCL20, CCL4, BCL10, CXCL5, TNFRSF21, PTX3, CCL13, IFNL1, CCL8, CCL25, CXCL11, CCL7, PF4, CCL26, SEMA7A, CXCL1, or a combination thereof, wherein the secretome induces an immune response.

Provided herein is a composition that comprises a) about 0.1% or more w/w of a secretome and b) a pharmaceutically acceptable excipient, wherein the secretome comprises: a. NID1, DKK1, DKK3, or a combination thereof, wherein the secretome induces cell-cell signaling; or b. XCL1, IGFBP3, TGFβ1, CCL5, CCL20, FSTL1, CCL4, BCL10, LGALS3, CXCL5, FST, PDGFB, CCL13, NID1, CCL8, IGFBP6, CCL25, IGFBP2, FSTL3, IGFBP4, CXCL11, GPC1, CCL7, DKK3, PF4, GDF15, TF, CCL26, SEMA7A, DKK1, INFRSF10C, CXCL1, or a combination thereof, wherein the secretome induces signal transduction.

Provided herein is a composition that comprises a) about 0.1% or more w/w of a secretome and b) a pharmaceutically acceptable excipient, wherein the secretome comprises: a. FURIN, wherein the secretome is for use in treating Alzheimer disease via the amyloid secretase pathway; b. FSTL1, FURIN, MMP1, or a combination thereof, wherein the secretome is for use in treating Alzheimer disease via the presenilin pathway; c. PDGFB, ANGPT1, TF, or a combination thereof, wherein the secretome is for use in treating angiogenesis; d. BIRC2, FAS, TNFRSF1A, INFRSF10C, or a combination thereof, wherein the secretome is for use in inducing or signaling apoptosis; e. CXCL12, wherein the secretome induces axon guidance mediated by Slit/Robo; f. UNC5C, wherein the secretome induces axon guidance mediated by netrin; g. SERPINE1, PLAUR, GP1BA, THBD, KLK3, TF, or a combination thereof, wherein the secretome induces blood coagulation; h. BIRC2, SERPINE1, CLU, CXCL1, or a combination thereof, wherein the secretome is for CCKR signaling; i. FSTL1, wherein the secretome induces cadherin signaling; j. FURIN, wherein the secretome induces the endothelin signaling pathway; k. FAS, wherein the secretome induces the FAS signaling pathway; 1. TGFβ1, MIF, FST, INS, or a combination thereof, wherein the secretome induces the gonadotropin-releasing hormone receptor pathway; m. IL6, CCL5, CCL20, CCL4, CCL13, CCL8, CCL7, PF4, CCL26, or a combination thereof, wherein the secretome induces inflammation mediated by a chemokine or a cytokine signaling pathway; n. insulin (INS), wherein the secretome induces the MAPKK/MAPK cascade; o. insulin (INS), wherein the secretome induces the PKB signaling cascade; p. IL6, IL21, or a combination thereof, wherein the secretome induces an interleukin signaling pathway; q. PDGFB, wherein the secretome induces a PDGF signaling pathway; r. SEREPIN1, PLAUR, MMP1, or a combination thereof, wherein the secretome induces a plasminogen activating cascade; s. B2M, wherein the secretome induces T cell activation; t. TGFB1, GDF15, or a combination thereof, wherein the secretome induces a TGF-beta signaling pathway; u. TLR3, wherein the secretome induces a Toll receptor signaling pathway; v. FSTL1, wherein the secretome induces a Wnt signaling pathway; or w. IGFBP3, SEREPINE1, FAS, THBS1, or a combination thereof, wherein the secretome induces the p53 pathway.

Provided herein is a method, comprising administering to a subject in need thereof any of any of such compositions. Provided herein is a use of any of any of such compositions for treating a subject in need thereof, or for the manufacture of a medicament for treating a subject in need thereof. Provided herein is a use of any of any of such compositions, for use in an in vitro culture or assay.

In any of such compositions, the composition can be substantially free from a cell. Alternatively, the composition can be free of cells.

The can be present in the composition in an amount of from about 0.1% to about 75% by weight, from about 0.1% to about 65% by weight, from about 0.1% to about 50% by weight, from about 0.1% to about 40% by weight, from about 0.1% to about 30% by weight, from about 0.1% to about 20% by weight, from about 0.1% to about 15% by weight, from about 0.1% to about 10% by weight, or from about 0.1% to about 5% by weight.

In any of such embodiments, the secretome can comprise at least: 0.6%, 1%, 1.25%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% of the composition. In some instances, the secretome comprises about 0.6%, about 1%, about 1.25%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of the composition. In some instances, the secretome comprises from about 0.6% to about 25% of the composition, or from about 2.5% to about 10% of the composition.

When a secretome contains more than one protein, each protein can be present in a ratio of from about 1:1 to about 20:1. For example, each protein can be present in a ratio of about 1:1, about 2:1, about 3:1, about 4:1, about 5: 1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1.

In some cases, a composition disclosed herein can be aseptic. In some cases, the composition can comprise one or more resident microbes or cells. The one or more microbes or cells can be viruses, bacteria, eukaryotic cells, or any combination thereof. In some instances, the one or more microbes or cells may not be pathogenic. In some instances, the composition can comprise a bacterium or bacteria at a concentration of less than 10 colony forming units (CFU)/gram (g), 50 CFU/g, 100 CFU/g, 150 CFU/g, 200 CFU/g, 300 CFU/g, 400 CFU/g, 500 CFU/g, 600 CFU/g, 700 CFU/g, 800 CFU/g, 900 CFU/g, or 1000 CFU/g. In some cases, the composition can comprise bacteria at a concentration of from about 10 CFU/g to about 1000 CFU/g, from about 10 CFU/g to about 50 CFU/g, from about 20 CFU/g to about 100 CFU/g, from about 50 CFU/g to about 200 CFU/g, from about 100 CFU/g to about 250 CFU/g, from about 200 CFU/g to about 500 CFU/g, from about 500 CFU/g to about 700CFU/g, or from about 600 CFU/g to about 1000 CFU/g. In some instances, the composition may be substantially free of, or free of, Staphylococcus aureus, Streptococcus pyogenes, Pseudomonas aeruginosa, Pseudomonas species, Klebsiella pneumoniae, or any combination thereof.

In some cases, a composition disclosed herein may not contain a heavy metal such as, for example, lead, bithionol, chlorofluorocarbon propellants, nitrosamines, chloroform, halogenated salicylanilides, hexachlorophene, mercury compounds, 1,4-dioxane, methylene chloride, prohibited cattle materials, sunscreen compounds, vinyl chloride, zirconium-containing complexes, or any combination thereof. In some instances, the prohibited cattle materials can comprise the brain, skull, eyes, trigeminal ganglia, spinal cord, vertebral column, dorsal root ganglia, tonsils, distal ileum of the small intestine or any combination thereof. In some instances, the composition may comprise lead at levels of 10 (parts per million) ppm or less.

In some cases, a composition herein does not comprise a color additive. In some cases, the composition can comprise a color additive. In some cases, the composition may contain an incidental ingredient such as a color additive in an insignificant level in the composition, for example less than: 5%, 4%, 3%, 2%, 1%, 0.5%, or 0.1%. In some cases, the incidental ingredient may have no technical/structural, functional or any combination thereof effect in the composition, e.g., an incidental ingredient is not an active ingredient.

In some instances, the composition can comprise a protein at a concentration of less than 1 nanogram/milliliter (ng/ml), 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml, 20 ng/ml, 21 ng/ml, 22 ng/ml, 23 ng/ml, 24 ng/ml, 25 ng/ml, 30 ng/ml, 35 ng/ml, 40 ng/ml, 45 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, or 10000 ng/ml. In some instances, the composition can comprise a protein at a concentration of: from about 1 ng/ml to about 100 ng/ml, from about 10 ng/ml to about 200 ng/ml, from about 10 ng/ml to about 400 ng/ml, from about 50 ng/ml to 300 ng/ml, from about 100 ng/ml to about 200 ng/ml, from about 150 ng/ml to about 400 ng/ml, from about 200 ng/ml to about 600 ng/ml, from about 400 ng/ml to about 700 ng/ml, from about 500 ng/ml to about 900 ng/ml, from about 600 ng/ml to about 1000 ng/ml, from about 900 ng/ml to about 1500 ng/ml, or from about 1000 ng/ml to about 10000 ng/ml.

In some instances, a secretome can comprise at least 0.01%, 0.1%. 1%, 1.25%, 2%, 3%, 4%, 5%, 6%, 7%, 8%, 9%, 10%, 20%, 30%, 40%, 50%, 60%, 70%, 80%, 90%, 95%, or 99% of the composition. In some instances, the secretome can comprise from about: 0.01% to about 0.1% of the composition, from about 0.01% to about 1% of the composition, from about 1% to about 2% of the composition, from about 1% to about 5% of the composition, from about 3% to about 8% of the composition, from about 5% to about 10% of the composition, from about 10% to about 20% of the composition, from about 20% to about 40% of the composition, from about 30% to about 50% of the composition, from about 50% to about 75% of the composition, from about 60% to about 90% of the composition, from about 75% to about 95% of the composition, or from about 80% to about 99% of the composition.

In some cases, a composition described herein can comprise an exosome, a liposome, a nanoparticle, or any combination thereof. In some instances, a liposome can be in a form of a nanoparticle. In some instances, a nanoparticle can comprise a liposome. In some cases, the exosome, the liposome, the nanoparticle, or any combination thereof, can comprise the secretome, a phospholipid, a protein, a hydrophilic active agent, a hydrophilic active agent, a vitamin, an inactive ingredient, or any combination thereof. The liposome can include, but may not be limited to, a unilamellar liposome, a multilamellar liposome, an archaeosome, a noisome, a novasome, a cryptosome, an emulsome, a vesosome, a nanoliposome, a nanoemulsion, or a derivative of any of these, or any combination thereof. The nanoparticle can include, but may not be limited to, a biopolymeric nanoparticle, an alginate nanoparticle, a xanthan gum nanoparticle, a cellulose nanoparticle, a lipid nanoparticle, a dendrimer, a polymeric micelle, a polyplexed, an inorganic nanoparticle, a nanocrystal, a metallic nanoparticle, a quantum dot, a protein nanoparticle, a polysaccharide nanoparticle, a derivative of any of these, or any combination thereof.

In some instances, a nanoparticle can be less than 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm. In some instances, the nanoparticle can be more than 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 35 nm, 40 nm, 45 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm. In some instances, the nanoparticles can have an average particle size of from about 1 nm to about 100 nm, from about 10 nm to about 200 nm, from about 10 nm to about 400 nm, from about 50 nm to 300 nm, from about 100 nm to about 200 nm, from about 150 nm to about 400 nm, from about 200 nm to about 600 nm, from about 400 nm to about 700 nm, from about 500 nm to about 900 nm, from about 600 nm to about 1000 nm, or from about 700 nm to about 1500 nm.

### EXAMPLES

The application may be better understood by reference to the following nonlimiting examples, which are provided as exemplary embodiments of the application. The following examples are presented in order to more fully illustrate embodiments and should in no way be construed, however, as limiting the broad scope of the application.

### Example 1. Secretome Composition Profiles

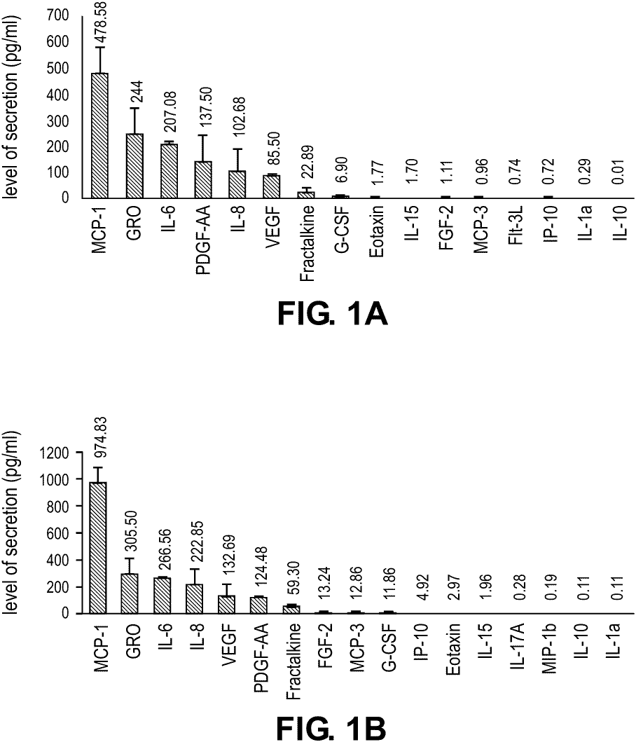

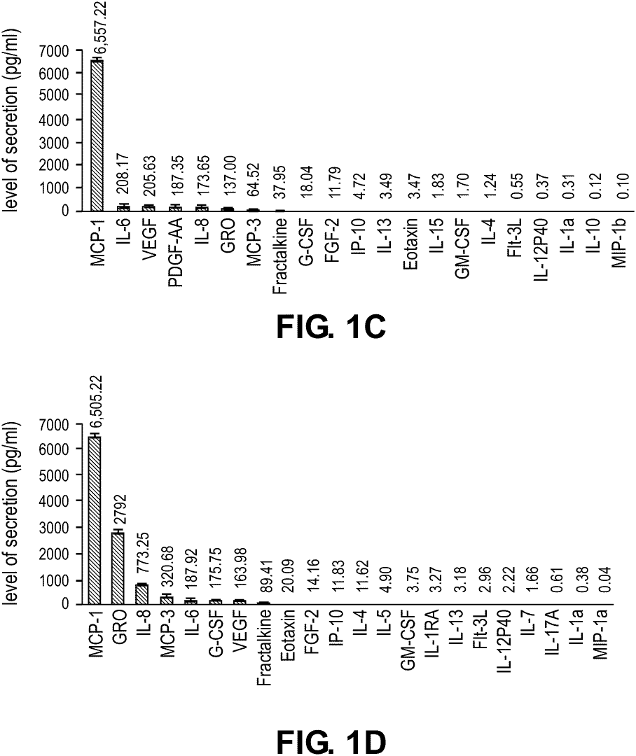

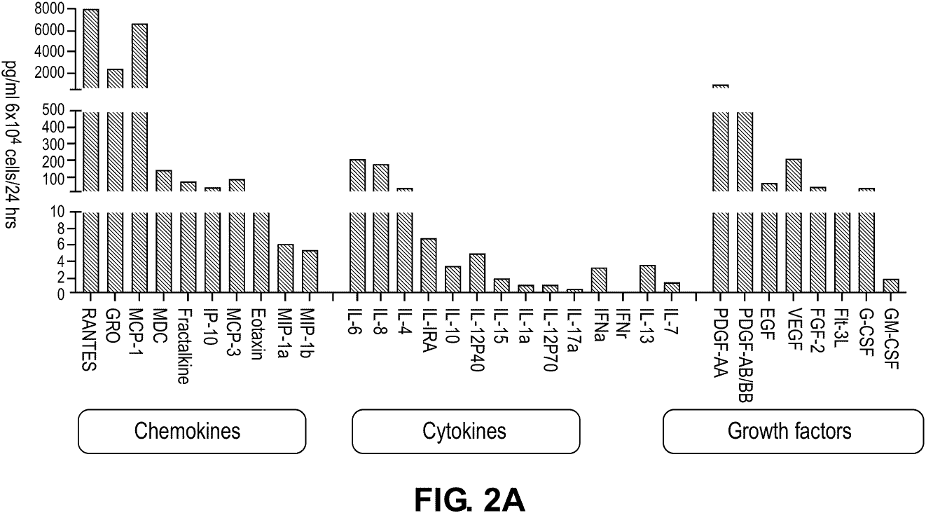

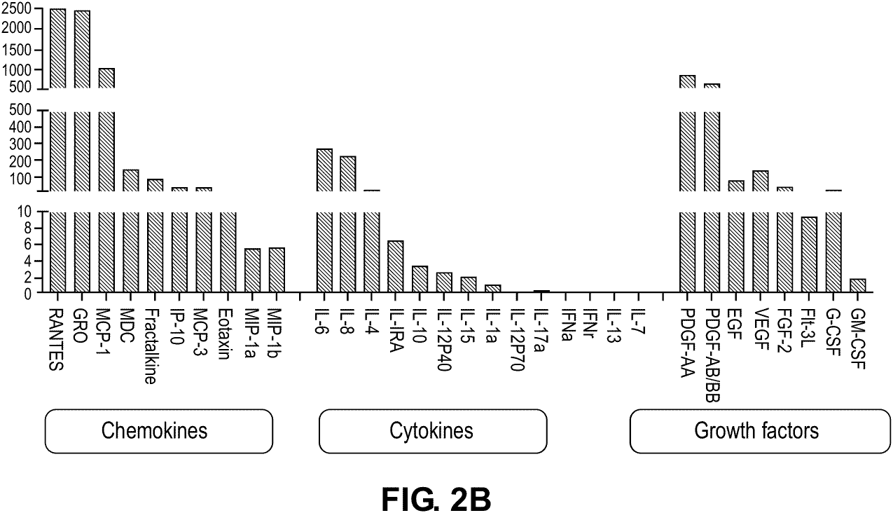

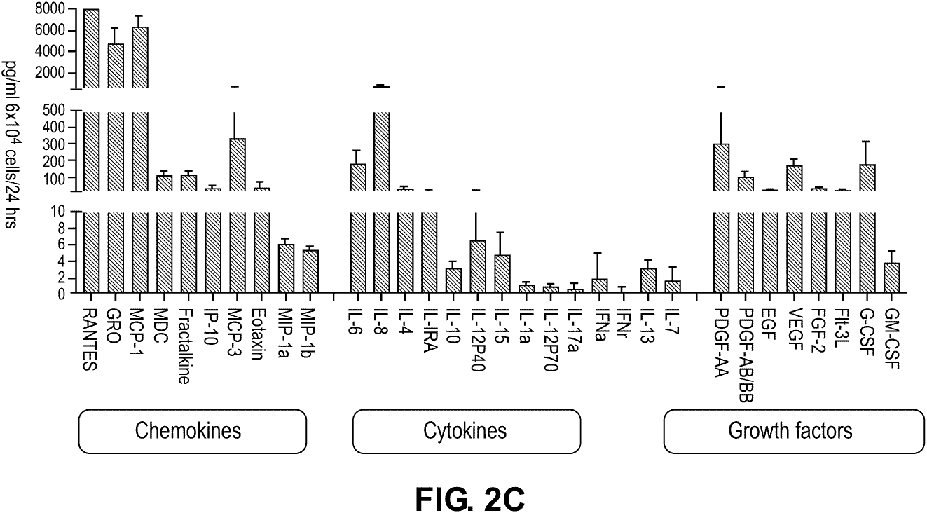

A MILLIPLEX MAP Human Cytokine/Chemokine Magnetic Bead Panel (Premixed 41 Plex-Immunology Multiplex Assay) was used to determine the composition of the secretomes from various stem cell culture extracts. The concentrations were measured with a Luminex LX200, and the results are shown in FIGS. 1A-1D and FIGS. 2A-2C. The tested secretome analytes include sCD40L, EGF, Eotaxin/CCL11, FGF-2, Flt-3 ligand, Fractalkine, G-CSF, GM-CSF, GRO, IFN-α2, IFN-γ, IL-1α, IL-1β, IL-1Rα, IL-2, IL-3, IL-4, IL-5, IL-6, IL-7, IL-8, IL-9, IL-10, IL-12 (p40), IL-12 (p70), IL-13, IL-15, IL-17A, IP-10, MCP-1, MCP-3, MDC (CCL22), MIP-1α, MIP-1β, PDGF-AA, PDGF-AB/BB, RANTES, TGF-α, TNF-α, TNF-β, and VEGF. MCP-1 was found at high levels in the secretome. MCP-1 was present in a significant portion of the secretome to use in a formulation as described herein. Many other secretome proteins were also identified in high concentrations, such as GRO, IL-6, PDFG-AA, IL-8, MCP-3, and VEGF. These secretome proteins may be used in a formulation as described herein.

### Example 2. In Vitro Activity Assay of Secretome Formulations

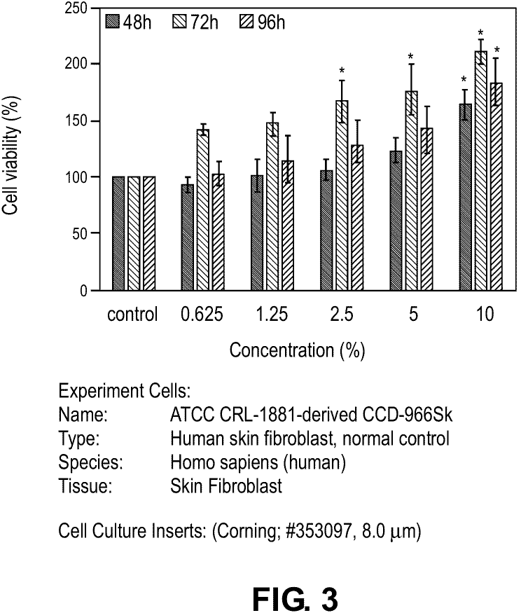

An MTT assay is a colorimetric assay used to determine cellular viability by assessing cellular metabolic activity. ATCC CRL-1881-derived CCD-966SK human skin fibroblast cells were grown with control, 0.625%, 1.25%, 2.5%, 5%, or 10% of exemplary secretome formulations disclosed herein at 37° C. for 48, 72, or 96 hours. 150 µl MTT (0.5 mg/ml) were used for reading with ELISA reader at 595 nm, and the results are shown in FIG. 3. At secretome concentrations of more than 2.5% and treatment of 72 hours or longer, there were significantly increased growth of the skin cells. The increased growth suggests that the secretome can increase the growth of facial skin cells, which may decrease aging symptoms, such as, wrinkles and age spots. Additionally, the enhanced growth rate suggests the secretome can be used to increase the speed of wound healing.

### Example 3. MCP-1 Induced Increased Migration of Skin Cells in Vitro

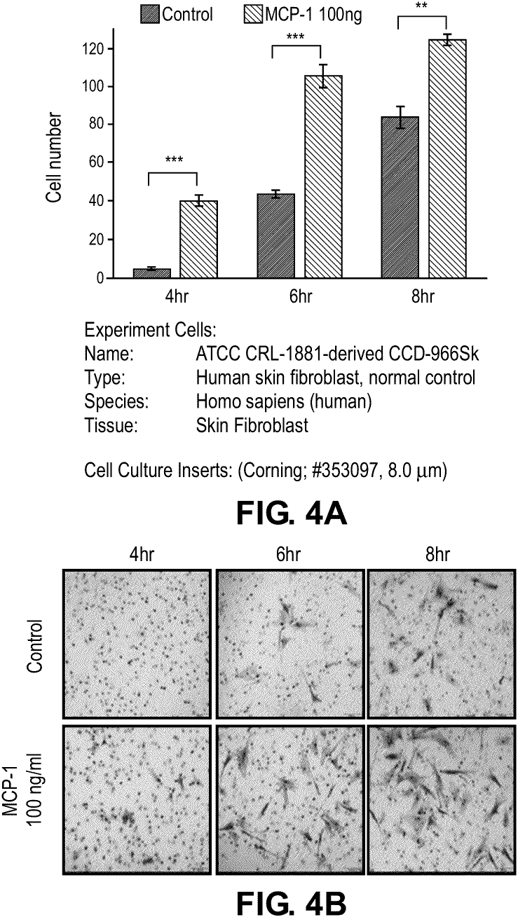

A transwell migration assay was used to study the migratory response of cells to different inducers and inhibitors. ATCC CRL-1881-derived CCD-966SK human skin fibroblast cells were exposed to the presence of MCP-1 (100 ng/ml), and transwell migration was determined after 4, 6, and 8 hours and compared to control (no MCP-1). When MCP-1 was present in the transwell assay, there was an increase in the migration of the tested skin cells, as shown in FIG. 4A. Results were statistically significant (** p < 0.01, *** p < 0.001). The images of the cells in FIG. 4B for the cultures of FIG. 4A show MCP-1 induced increased migration of skin cells. The increased migration is useful for wound healing and fullness of skin. MCP-1 may be added to a composition described herein to, at least in part, reduce aging symptoms, such as, wrinkles and age spots.

### Example 4. Cosmetic Composition With Nanoparticle Delivery

A cosmetic composition comprises nanoparticles carrying a lipid and secretome and other cosmetically acceptable ingredients. Secretome proteins such as MCP-1, CXCL2, IL6, IL-8, and VEGF may be incorporated into the nanoparticle as passenger molecules, for example using the methods described herein. The nanoparticles may comprise amphipathic, lipophilic, and hydrophilic passenger molecules. The nanoparticle may be an assembly of phospholipids that can encapsulate an active agent or multiple active agents. The nanoparticle carrying the secretome may be combined with a carrier solution, for example glycerol and/or water. The cosmetic formulation also may contain vitamin B3 and vitamin A to potentially increase its effectiveness. Additional non-active ingredients can be added to form a cream. The cream can be packaged in a plastic vial. A subject can apply the cream to their face to prevent and reduce the presence of wrinkles. Alternatively, non-active ingredients can be added to the formulation to form a solution. The solution can be applied to a patch. The patch can be packaged in a sealed container to prevent the cosmetic formulated patch from drying out. A subject can apply the patch to their face to prevent age related wrinkles.

### Example 5. Cosmetic Compositions With Liposome Carrier

A cosmetic composition is formulated with a nanoliposome carrier that encapsulate secretome disclosed herein, for example proteins MCP-1, CXCL2, IL-6, IL-8, and VEGF. The nanoliposome can be formed using the methods described herein. The nanoliposome can be combined with a filler solution of saline and hyaluronic acid. Additional non-active ingredients can be added to form the injectable solution. The injection can be packaged in a disposable dispenser or delivery tool, such as a syringe. A medical professional can treat the subject with an intradermal injection comprising the nanoliposome composition to reduce the presence of wrinkles.

Another cosmetic composition is formed with a unilamellar liposome carrier that encapsulate secretome disclosed herein. The unilamellar liposome can comprise secretome proteins, such as MCP-1, CXCL2, IL-6, IL-8, and VEGF. The unilamellar liposome can be formed using the methods described herein. The unilamellar liposome comprising the secretome proteins can be combined with linoleic acid, and a carrier solution (e.g. polyethylene glycol). Additional non-active ingredients can be added to form a butter. The butter can be packaged in a sealed container to prevent the cosmetic formulation from drying out. A cosmetic professional can apply the butter to the face of the subject to reduce the effects of skin aging.

### Example 6. Regenerative Medicine

A composition herein is used for regenerative medicine. For instance, a pharmaceutical formulation is formulated with the secretome disclosed herein to treat liver failure. The pharmaceutical composition may be formulated with secretome proteins, for example HEGF (heparin binding epidermal growth factor), EGF (epidermal growth factor), HGF (hepatocyte growth factor), MCP-1, CXCL2 (GRO), VEGF, PDGF(e.g., PDGF-AA), IL-6, IL-8, or any combination thereof. The composition comprises a pharmaceutically acceptable excipient, for example saline or a phosphate buffer. The pharmaceutical composition can be injected intravenously (IV) to treat liver failure. The IV administration may occur daily. The secretome proteins may act as regenerative signals and promote liver healing in a subject. The treatment can reverse liver failure, for example, reducing liver scaring, fat deposits in the liver, liver cirrhosis, or any combination thereof.

Another pharmaceutical formulation is formulated with the secretome disclosed herein and administered to subject suffering a stroke. The pharmaceutical composition may be formulated with secretome proteins, for example VEGF, EGF, NGF (neural growth factor), MCP-1, CXCL2 (GRO), PDGF (e.g., PDGF-AA), IL-6, IL-8, or any combination thereof. The composition comprises a pharmaceutically acceptable excipient, for example saline or a phosphate buffer. The pharmaceutical composition can be injected intravenously following a stroke in a subject. The pharmaceutical composition may be administered multiple times. The secretome proteins may act as regenerative signals and promote cerebral healing in a subject. The treatment can reverse stoke effects for example, improving speech, coordination, cognition, or any combination thereof.

Another pharmaceutical formulation is formulated with the secretome disclosed herein and administered to subject during surgery. The pharmaceutical composition may be formulated with secretome proteins, for example VEGF, EGF, PDGF, MCP-1, CXCL2 (GRO), PDGF (e.g., PDGF-AA), IL-6, IL-8, or any combination thereof. The composition comprises a pharmaceutically acceptable excipient, for example saline or a phosphate buffer. The pharmaceutical composition can be applied by a spray to surgically wound. The secretome proteins may act as regenerative signals and promote wound healing. The treatment may enhance wound healing, prevent surgical site infections, or a combination thereof.

### Example 7. hTSC Cell Culture and Secretome Analysis

Human trophoblastic stem cells (hTSC) cell lines (i.e., hTSC cell line 1, hTSC cell line 2, and hTSC cell line 3) were grown in a nutritional media (e.g., MESENCULT® with a cell attachment substrate) until confluency was reached (e.g., from about 3000 cells/cm² to about 9000 cells/cm², or about 6000 cells/cm²). Cells were washed and the media was replaced without Supplement. Hypoxia was induced in a chamber (e.g., culture for about 24 hours in a 2% 0₂ gas mixture). Medium was collected and frozen until use. Media from all three cell lines was tested using a QUANTIBODY® Human Kiloplex Array (RAYBIOTECH® Life, Inc.) to quantitatively analyze 1000 proteins. Experiments for hTSC cell line 1 and hTSC cell line 2 were repeated. Briefly, samples were processed and analyte concentration (pg/mL) were determined and compared to standard curves. Data was determined as % samples below the Limit of Detection (LOD), % samples above LOD, but <3xLOD, % samples in Best Confidence Interval, and % samples above maximum.

Selected analytes with concentration values ≥ 80% in Best Confidence Interval. 104 proteins were identified, grouped, and analyzed with respect to protein class, biologic processes, and/or pathways using Gene Ontology Analysis. Results from the hypoxia studies are provided below in Table 1. Abbreviations: hTSC C1-exp. 1 = hTSC cell line 1, experiment 1; hTSC C1-exp. 2 = hTSC cell line 1, experiment 2; hTSC C2-exp. 1 = hTSC cell line 2, experiment 1; hTSC C2-exp. 2 = hTSC cell line 2, experiment 2; hTSC C3 = hTSC cell line 3.

TABLE 1

| (pg/ml) | hTSC C1-exp. 1 | hTSC C1-exp. 2 | hTSC C2-exp. 1 | hTSC C2-exp. 2 | hTSC C3 | LOD | MAX |
|---|---|---|---|---|---|---|---|
| 6Ckine | 0.0 | 0.0 | 0.0 | 0.0 | 0.0 | 387.4 | 40,000.0.00 |
| Axl | 67.7 | 47.6 | 76.2 | 51.2 | 74.9 | 25.4 | 1,333.3.00 |
| BTC | 18.9 | 16.6 | 23.4 | 8.8 | 49.4 | 14.7 | 2,222.2.00 |
| CCL28 | 860.0 | ,653.0 | 981.3 | 543.3 | 1,210.1.00 | 19.7 | 40,000.0.00 |
| CTACK | 1,382.9.00 | 1,455.2.00 | 1,955.2.00 | 165.0 | 1,338.6.00 | 517.1 | 16,666.7.00 |
| CXCL16 | 0.0 | 20.8 | 52.4 | 10.8 | 11.8 | 6.9 | 2,222.2.00 |
| ENA-78 | 144.1 | 194.6 | 37.4 | 73.7 | 76.5 | 1.1 | 1,111.1.00 |
| Eotaxin-3 | 1,131.3.00 | 2,292.0.00 | 3,416.2.00 | 719.5 | 800.4 | 24.2 | 20,000.0.00 |
| GCP-2 | 0.4 | 1.5 | 0.0 | 1.7 | 0.3 | 0.1 | 370.4 |
| GRO | 0.9 | 0.7 | 0.0 | 0.0 | 0.0 | 6.1 | 1,000.0.00 |
| HCC-1 | 0.0 | 16.3 | 3.4 | 5.5 | 8.4 | 6.6 | 4,000.0.00 |
| HCC-4 | 0.7 | 1.7 | 1.4 | 0.7 | 1.9 | 0.6 | 370.4 |
| IL-9 | 1,728.4.00 | 1,363.4.00 | 2,729.3.00 | 533.7 | 1,645.3.00 | 903.5 | 66,666.7.00 |
| IL-17F | 0.0 | 7.3 | 4.4 | 0.0 | 2.6 | 18.4 | 11,111.1.00 |
| IL-18 BPα | 154.5 | 389.4 | 0.0 | 0.0 | 0.0 | 415.9 | 20,000.0.00 |
| IL-28A | 22.6 | 48.0 | 53.2 | 27.5 | 80.0 | 8.0 | 3.3 |
| IL-29 | 2,055.7.00 | 4,220.1.00 | 2,507.2.00 | 2,372.0.00 | 1,819.9.00 | 355.0 | 100,000.0.00 |
| IL-31 | 0.0 | 40.4 | 5.6 | 0.0 | 0.7 | 15.0 | 13,333.3.00 |
| IP-10 | 0.4 | 0.0 | 20.6 | 0.0 | 6.3 | 2.9 | 3,333.3.00 |
| I-TAC | 949.6 | 1,433.0.00 | 1,056.8.00 | 1,292.1.00 | 1,815.1.00 | 31.9 | 10,000.0.00 |
| LIF | 64.9 | 65.5 | 41.8 | 98.1 | 117.7 | 57.0 | 13,000.0.00 |
| LIGHT | 10.1 | 0.0 | 0.0 | 0.0 | 0.0 | 61.4 | 10,000.0.00 |
| Lymphotactin | 1,937.2.00 | 2,114.0.00 | 2,516.8.00 | 2,000.5.00 | 2,366.1.00 | 11.9 | 33,333.3.00 |
| MCP-2 | 37.3 | 67.7 | 94.2 | 25.6 | 67.5 | 0.9 | 2,000.0.00 |
| MCP-3 | 202.6 | 361.5 | 296.2 | 99.4 | 206.9 | 1.5 | 1,333.3.00 |
| MCP-4 | 4.4 | 5.7 | 19.2 | 4.3 | 16.6 | 1.3 | 1,111.1.00 |
| MDC | 0.0 | 2.0 | 0.0 | 0.1 | 0.0 | 4.3 | 3,333.3.00 |
| MIF | 339.7 | 455.7 | 250.0 | 1,074.5.00 | 35.9 | 23.5 | 4,000.0.00 |
| MIP-3a | 27.2 | 47.1 | 71.1 | 24.2 | 32.3 | 0.2 | 444.4 |
| MIP-3b | 19.8 | 12.2 | 6.7 | 11.3 | 23.6 | 14.2 | ,666.7 |
| MPIF-1 | 52.5 | 25.2 | 35.8 | 30.3 | 42.4 | 16.1 | 3,333.3.00 |
| MSP | 131.2 | 68.1 | 34.3 | 120.7 | 78.8 | 26.4 | 11,111.1.00 |
| NAP-2 | 3.4 | 1.7 | 2.2 | 1.8 | 2.5 | 0.4 | 148.1 |
| OPN | 589.1 | 369.0 | 1,652.3.00 | 562.0 | 523.5 | 22.9 | 33,333.3.00 |
| PARC | 1.4 | 2.0 | 1.6 | 1.1 | 2.6 | 1.4 | 148.1 |
| PF4 | 1,955.9.00 | 2,080.9.00 | 1,698.9.00 | 1,986.5.00 | 1,807.1.00 | 455.7 | 33,333.3.00 |
| SDF-1a | 131.5 | 88.4 | 131.6 | 105.2 | 139.2 | 1.4 | 370.4 |
| TARC | 0.0 | 0.0 | 2.8 | 0.0 | 0.0 | 4.7 | 3,333.3.00 |
| TECK | 89.6 | 56.2 | 77.0 | 84.9 | 106.6 | 11.7 | 33,333.3.00 |
| TSLP | 1.8 | 15.7 | 6.6 | 9.0 | 6.0 | 2.7 | 1,111.1.00 |
| Activin A | 66.9 | 13.6 | 25.4 | 13.0 | 65.3 | 13.0 | 100,000. 0.00 |
| AgRP | 0.0 | 0.0 | 0.0 | 0.0 | 0.0 | 6.6 | 3,333.3.00 |
| Angiogenin | 187.2 | 216.0 | 161.1 | 283.1 | 135.6 | 2.4 | 2,000.0.00 |
| ANG-1 | 671.2 | 1,015.3.00 | 573.9 | 757.6 | 419.4 | 16.3 | 40,000.0.00 |
| Angiostatin | 0.0 | 0.0 | 0.0 | 0.0 | 0.0 | 1,374.7.00 | 333,333.3.00 |
| Cathepsin S | 0.0 | 1.7 | 22.7 | 8.9 | 18.5 | 5.5 | 3,333.3.00 |
| CD40 | 0.0 | 0.0 | 0.0 | 0.0 | 0.0 | 8.5 | 10,000.0.00 |
| Cripto-1 | 0.0 | 0.0 | 0.0 | 0.0 | 0.0 | 1.9 | 10,000.0.00 |
| DAN | 0.0 | 0.0 | 0.0 | 15.0 | 0.0 | 52.9 | 40,000.0.00 |
| DKK-1 | 1,204.8.00 | 946.7 | 1,093.0.00 | 1,867.4.00 | 389.2 | 31.2 | 26,666.7.00 |
| E-Cadherin | 0.0 | 0.0 | 0.0 | 0.0 | 0.0 | 50.7 | 2,963.0.00 |
| EpCAM | 0.0 | 0.0 | 0.0 | 0.0 | 0.0 | 4.3 | 6,666.7.00 |
| FAS L | 0.0 | 0.0 | 0.0 | 0.0 | 0.0 | 4.7 | 2,000.0.00 |
| Fcg RIIBC | 0.0 | 0.0 | 0.0 | 0.0 | 0.0 | 3.0 | 10,000.0.00 |
| Follistatin | 959.3 | 419.9 | 906.0 | 2,214.7.00 | 128.0 | 66.5 | 40,000.0.00 |
| Galectin-7 | 0.0 | 0.0 | 0.0 | 10.7 | 0.0 | 50.4 | 33,333.3.00 |
| ICAM-2 | 0.0 | 277.8 | 0.0 | 8.1 | 4.6 | 60.5 | 33,333.3.00 |
| IL-13 R1 | 45.8 | 0.0 | 37.5 | 28.9 | 81.4 | 134.8 | 10,000.0.00 |
| IL-13 R2 | 0.0 | 20.5 | 0.0 | 0.0 | 0.0 | 110.0 | 20,000.0.00 |
| IL-17B | 224.8 | 0.0 | 0.0 | 13.7 | 0.0 | 259.1 | 40,000.0.00 |
| IL-2 Ra | 0.0 | 0.0 | 0.0 | 0.0 | 0.0 | 6.4 | 3,333.3.00 |
| IL-2 Rb | 0.0 | 28.7 | 73.6 | 0.0 | 0.0 | 99.8 | 11,111.1.00 |
| IL-23 | 0.0 | 0.0 | 0.0 | 0.0 | 0.0 | 27.8 | 40,000.0.00 |
| LAP(TGFb1) | 336.7 | 110.3 | 32.0 | 373.4 | 62.1 | 19.8 | 4,000.0.00 |
| NrCAM | 0.0 | 0.0 | 0.0 | 0.0 | 0.0 | 57.4 | 20,000.0.00 |
| PAI-1 | ,335.7 | 2,806.9.00 | 3,275.1.00 | 4,244.2.00 | 4,202.5.00 | 17.9 | 1,481.5.00 |
| PDGF-AB | 0.0 | 0.0 | 0.0 | 0.0 | 0.0 | 8.3 | 10,000.0.00 |
| Resistin | 0.0 | 0.0 | 0.0 | 0.0 | 0.0 | 9.5 | 6,666.7.00 |
| SDF-1b | 1.8 | 3.1 | 0.0 | 0.0 | 0.0 | 7.1 | 4,444.4.00 |
| gp 130 | 392.8 | 111.1 | 589.9 | 0.0 | 0.0 | 311.1 | 6,666.7.00 |
| Shh-N | 0.0 | 3.4 | 0.0 | 2.5 | 0.0 | 2.9 | 4,444.4.00 |
| Siglec-5 | 0.0 | 0. 7 | 0.0 | 29.9 | 0.0 | 15.0 | 10,000.0.00 |
| IL-1 R4 | 0.0 | 0.0 | 0.0 | 0.0 | 0.0 | 9.2 | 1,333.3.00 |
| TGFb2 | 0.0 | 0.0 | 0.0 | 0.0 | 0.0 | 123.6 | 40,000.0.00 |
| Tie-2 | 0.0 | 9.6 | 0.0 | 0.0 | 0.0 | 21.9 | 10,000.0.00 |
| TPO | 20.0 | 35.0 | 14.9 | 0.0 | 5.7 | 28.1 | 200,000. 0.00 |
| TRAIL R4 | 0.0 | 0.0 | 0.0 | 0.0 | 0.0 | 14.0 | 8,000.0.00 |
| TREM-1 | 0.0 | 0.0 | 0.0 | 7.9 | 0.0 | 30.7 | 6,666.7.00 |
| VEGF-C | 0.0 | 0.0 | 0.0 | 0.0 | 0.0 | 7.2 | 6,666.7.00 |
| VEGF R1 | 1,872.7.00 | 568.7 | 900.9 | 1,445.8.00 | 314.3 | 158.4 | 40,000.0.00 |
| Adiponectin | 0.0 | 0.0 | 0.0 | 0.0 | 0.0 | 68.9 | 100,000. 0.00 |
| Adipsin | 0.0 | 0.0 | 0.0 | 0.0 | 0.0 | 1.5 | 740.7 |
| AFP | 0.0 | 11.9 | 0.0 | 44.9 | 0.0 | 64.2 | 3,333.3.00 |
| ANGPTL4 | 30.3 | 35.2 | 62.5 | 113.3 | 76.6 | 7.3 | 14,814.8.00 |
| B2M | 284.3 | 195.3 | 107.2 | 310.9 | 420.1 | 0.1 | 370.4 |
| BCAM | 0.0 | 0.0 | 0.0 | 0.0 | 0.0 | 72.6 | 13,333.3.00 |
| CA125 | 526.3 | 637.0 | 482.8 | 806.2 | 859.5 | 183.0 | 100,000. 0.00 |
| CA15-3 | 0.0 | 0.0 | 0.0 | 16.3 | 0.0 | 84.5 | 10,000.0.00 |
| CEA | 0.1 | 2.2 | 0.0 | 6.3 | 0.9 | 7.4 | 740.7 |
| CRP | 0.0 | 0.0 | 0.0 | 0.0 | 0.0 | 10.1 | 370.4 |
| ErbB2 | 0.0 | 0.0 | 0.0 | 0.0 | 0.0 | 14.2 | 10,000.0.00 |
| Ferritin | 0.0 | 12,816. 8.00 | 7,978.2.00 | 70,401. 7.00 | 10,340. 1.00 | 17,686. 4.00 | 800,000. 0.00 |
| FSH | 0.0 | 29.7 | 0.0 | 57.7 | 18.6 | 40.6 | 10,000.0.00 |
| GROa | 6,090.7.00 | 6,529.1.00 | 1,181.9.00 | 4,795.8.00 | 302.3 | 181.8 | 100,000. 0.00 |
| hCGb | 0.0 | 0.0 | 0.0 | 0.0 | 0.0 | 100.2 | 20,000.0.00 |
| IGF-1R | 0.0 | 0.0 | 0.0 | 0.0 | 0.0 | 259.8 | 33,333.3.00 |
| IL-1 RII | 7.9 | 0.0 | 0.0 | 21.3 | 0.0 | 17.7 | 6,666.7.00 |
| IL-3 | 7.9 | 0.0 | 0.0 | 0.0 | 0.0 | 5.1 | 10,000.0.00 |
| IL-18 Rb | 0.0 | 0.0 | 0.0 | 0.0 | 0.0 | 18.7 | 20,000.0.00 |
| IL-21 | 162.2 | 196.4 | 240.7 | 295.4 | 251.4 | 42.5 | 33,333.3.00 |
| Leptin | 9.3 | 0.0 | 0.4 | 19.4 | 11.0 | 3.1 | 40,000.0.00 |
| MMP-1 | 5,574.7.00 | 519.6 | 7,596.2.00 | 426.8 | 8,190.5.00 | 15.0 | 40,000.0.00 |
| MMP-2 | 32.0 | 0.0 | 20.9 | 15.4 | 0.0 | 10.4 | 33,333.3.00 |
| MMP-3 | 0.0 | 0.0 | 0.0 | 0.0 | 5.4 | 14.5 | 13,333.3.00 |
| MMP-8 | 13.4 | 0.0 | 0.0 | 14.4 | 26.5 | 29.7 | 10,000.0.00 |
| MMP-9 | 0.0 | 0.0 | 0.0 | 1.9 | 0.0 | 13.9 | 2,222.2.00 |
| MMP-10 | 542.5 | 0.0 | 0.0 | 98.2 | 307.7 | 511.3 | 10,000.0.00 |
| MMP-13 | 0.0 | 0.0 | 0.0 | 22.1 | 5.9 | 11.6 | 10,000.0.00 |
| NCAM-1 | 0.0 | 0.0 | 0.0 | 6.3 | 0.0 | 83.0 | 200,000. 0.00 |
| Nidogen-1 | 11,292. 8.00 | 11,411. 1.00 | 7,442.4.00 | 18,034. 4.00 | 8,369.4.00 | 12.2 | 20,000.0.00 |
| NSE | 22.8 | 22.0 | 0.0 | 3.7 | 0.0 | 5.5 | 11,111.1.00 |
| OSM | 0.0 | 0.0 | 0.0 | 0.0 | 0.0 | 32.8 | 3,333.3.00 |
| Procalcitonin | 7.6 | 9.1 | 5.1 | 16.8 | 7.0 | 3.3 | 3,703.7.00 |
| Prolactin | 0.0 | 0.0 | 0.0 | 0.0 | 0.0 | 107.9 | 44,444.4.00 |
| PSA-free | 42.3 | 38.1 | 22.5 | 52.0 | 48.6 | 5.1 | 2,222.2.00 |
| Siglec-9 | 191.8 | 297.5 | 166.3 | 271.9 | 299.0 | 28.1 | 13,333.3.00 |
| TACE | 0.0 | 0.0 | 0.0 | 4.2 | 0.0 | 49.6 | 100,000. 0.00 |
| Thyroglobulin | 193.6 | 43.2 | 0.0 | 225.0 | 0.0 | 384.1 | 100,000. 0.00 |
| TIMP-4 | 47.4 | 53.3 | 21.4 | 134.6 | 5.8 | 3.6 | 20,000.0.00 |
| TSH | 7.4 | 3.1 | 13.3 | 117.9 | 36.6 | 57.5 | 6,666.7.00 |
| 2B4 | 14.6 | 6.3 | 3.9 | 0.0 | 10.1 | 14.3 | 10,000.0.00 |
| ADAM9 | 127.2 | 73.5 | 81.5 | 98.9 | 62.7 | 37.0 | 33,333.3.00 |
| ANG-2 | 36.4 | 16.4 | 0.0 | 0.0 | 22.1 | 24.9 | 20,000.0.00 |
| APRIL | 4,200.8.00 | 0.0 | 159.9 | 311.6 | 60.5 | 3,111.3.00 | 200,000. 0.00 |
| BMP-2 | 5.6 | 25.3 | 13.9 | 0.0 | 0.0 | 55.7 | 11,111.1.00 |
| BMP-9 | 11.2 | 0.0 | 0.0 | 0.0 | 12.4 | 22.1 | 4,000.0.00 |
| C5a | 25.2 | 21.6 | 7.4 | 8.7 | 17.0 | 20.9 | 10,000.0.00 |
| Cathepsin L | 67.5 | 65.7 | 23.4 | 39.3 | 33.1 | 14.0 | 10,000.0.00 |
| CD200 | 254.7 | 0.0 | 141.1 | 36.7 | 172.6 | 193.1 | 100,000. 0.00 |
| CD97 | 979.6 | 862.3 | 287.7 | 1,090.8.00 | 763.3 | 1,103.7.00 | 100,000. 0.00 |
| Chemerin | 7,391.9.00 | 489.5 | 1,880.0.00 | 236.0 | 2,109.9.00 | 190.5 | 66,666.7.00 |
| DcR3 | 3,112.5.00 | 2,894.0.00 | 1,458.1.00 | 0.0 | 0.0 | 4,228.0.00 | 66,666.7.00 |
| FABP2 | 24.4 | 0.0 | 0.0 | 4.8 | 0.7 | 21.2 | 33,333.3.00 |
| FAP | 125.0 | 131.9 | 122.2 | 345.1 | 177.6 | 21.8 | 6,666.7.00 |
| FGF-19 | 111.4 | 0.0 | 0.0 | 0.0 | 0.0 | 64.8 | 6,666.7.00 |
| Galectin-3 | 61.2 | 151.7 | 29.8 | 86.0 | 16.8 | 3.6 | 1,333.3.00 |
| HGF R | 107.4 | 30.1 | 44.0 | 79.9 | 37.3 | 5.7 | 1,333.3.00 |
| IFNab R2 | 3,395.3.00 | 3,140.4.00 | 2,293.1.00 | 1,704.0.00 | 232.2 | 3,013.4.00 | 33,333.3.00 |
| IGF-2 | 3,119.6.00 | 354.4 | 77.1 | 429.8 | 505.9 | 1,679.9.00 | 100,000. 0.00 |
| IGF-2R | 313.3 | 0.0 | 66.6 | 313.8 | 0.0 | 295.1 | 20,000.0.00 |
| IL-1 R6 | 9,944.9.00 | 957.9 | 0.0 | 0.0 | 0.0 | 0,295. 3.00 | 100,000. 0.00 |
| IL-24 | 94.1 | 8.8 | 0.0 | 5.0 | 38.5 | 86.0 | 33,333.3.00 |
| IL-33 | 31.2 | 8.4 | 6.7 | 15.7 | 18.6 | 9.8 | 10,000.0.00 |
| Kallikrein 14 | 6.3 | 0.0 | 0.2 | 0.0 | 4.4 | 5.2 | 4,000.0.00 |
| Legumain | 373.5 | 555.3 | 303.2 | 682.9 | 947.2 | 9.9 | 10,000.0.00 |
| LOX-1 | 1.9 | 1.1 | 2.5 | 2.4 | 2.1 | 0.8 | 666.7 |
| MBL | 3.5 | 0.0 | 0.0 | 1.5 | 1.6 | 2.2 | 1,000.0.00 |
| Neprilysin | 18.2 | 0.9 | 15.1 | 24.3 | 3.8 | 39.2 | 20,000.0.00 |
| Notch-1 | 3.4 | 0.0 | 0.0 | 0.5 | 0.0 | 14.7 | 4,000.0.00 |
| NOV | 17.0 | 0.0 | 0.0 | 5.0 | 11.0 | 7.8 | 4,000.0.00 |
| Osteoactivin | 21.0 | 0.0 | 0.0 | 0.0 | 0.0 | 19.6 | 10,000.0.00 |
| PD-1 | 0.0 | 0.0 | 0.0 | 0.0 | 2.3 | 11.9 | 4,000.0.00 |
| PGRP-S | 7.8 | 4.1 | 0.2 | 5.1 | 3.3 | 2.8 | 1,000.0.00 |
| Serpin A4 | 113.4 | 0.0 | 0.0 | 0.0 | 24.7 | 11.1 | 10,000.0.00 |
| sFRP-3 | 1,651.0.00 | 0.0 | 0.0 | 535.0 | 228.3 | 617.4 | 100,000. 0.00 |
| Thrombomoduli n | 1,973.2.00 | 1,960.9.00 | 1,519.3.00 | 2,227.1.00 | 1,905.2.00 | 2.0 | 3,703.7.00 |
| TLR2 | 13.1 | 0.0 | 0.0 | 11.4 | 3.5 | 47.9 | 20,000.0.00 |
| TRAIL R1 | 346.7 | 249.8 | 386.3 | 0.0 | 163.0 | 885.4 | 10,000.0.00 |
| Transferrin | 1,920.0.00 | 1,676.1.00 | 1,545.8.00 | 1,654.8.00 | 1,728.6.00 | 2.9 | 3,703.7.00 |
| WIF-1 | 35.0 | 16.4 | 16.1 | 12.7 | 24.8 | 12.3 | 20,000.0.00 |
| ACE-2 | 0.0 | 0.0 | 0.0 | 0.0 | 0.0 | 3,862.9.00 | 400,000. 0.00 |
| Albumin | 118.0 | 73.2 | 67.7 | 125.0 | 147.4 | 0.2 | 740.7 |
| AMICA | 0.0 | 0.0 | 0.0 | 0.0 | 0.0 | 27.4 | 20,000.0.00 |
| ANG-4 | 0.0 | 0.0 | 0.0 | 0.0 | 0.0 | 23.5 | 20,000.0.00 |
| BAFF | 0.0 | 0.0 | 0.0 | 0.0 | 0.0 | 0.9 | 3,333.3.00 |
| CA19-9 | 0.0 | 0.0 | 0.0 | 0.0 | 0.0 | 418.6 | 11,111.1.00 |
| CD163 | 0.0 | 0.0 | 0.0 | 0.0 | 0.0 | 36.1 | 66,666.7.00 |
| Clusterin | 36.6 | 41.7 | 21.8 | 41.6 | 0.0 | 5.6 | 3,333.3.00 |
| CRTAM | 0.0 | 0.0 | 0.0 | 0.0 | 0.0 | 10.9 | 4,000.0.00 |
| CXCL14 | 67,160. 0.00 | 55,114. 5.00 | 63,509. 2.00 | 65,810. 4.00 | 64,127. 6.00 | 121.6 | 3,703.7.00 |
| Cystatin C | 5,291.4.00 | 5,879.8.00 | 2,206.8.00 | 3,812.5.00 | 2,081.3.00 | 4.1 | 33,333.3.00 |
| Decorin | 2,081.2.00 | 2,170.1.00 | 1,919.0.00 | 2,341.4.00 | 1,582.6.00 | 1.1 | 2,000.0.00 |
| Dkk-3 | 35,988. 8.00 | 36,791. 9.00 | 41,043. 8.00 | 48,840. 7.00 | 40,862. 2.00 | 12.9 | 100,000. 0.00 |
| DLL1 | 0.0 | 0.0 | 0.0 | 0.0 | 0.0 | 6.9 | 20,000.0.00 |
| Fetuin A | 0.0 | 0.0 | 0.0 | 0.0 | 0.0 | 1,170.3.00 | 100,000. 0.00 |
| aFGF | 1,936.8.00 | 1,743.6.00 | 2,231.8.00 | 871.8 | 3,102.7.00 | 8,866.9.00 | 200,000. 0.00 |
| FOLR1 | 0.0 | 0.0 | 0.0 | 0.0 | 0.0 | 2.3 | 3,703.7.00 |
| Furin | 1,742.9.00 | 182.6 | 491.9 | 2,447.9.00 | 2,786.5.00 | 72.1 | 66,666.7.00 |
| GASP-1 | 0.0 | 0.0 | 0.0 | 0.0 | 0.0 | 2.9 | 2,000.0.00 |
| GASP-2 | 0.0 | 0.0 | 0.0 | 0.0 | 0.0 | 9.7 | 33,333.3.00 |
| G-CSF R | 0.0 | 0.0 | 0.0 | 0.0 | 0.0 | 1.5 | 3,333.3.00 |
| HAI-2 | 0.0 | 0.0 | 0.0 | 0.0 | 0.0 | 2.0 | 40,000.0.00 |
| IL-17B R | 0.0 | 0.0 | 0.0 | 0.0 | 0.0 | 61.1 | 100,000. 0.00 |
| IL-27 | 0.0 | 0.0 | 0.0 | 0.0 | 0.0 | 9.0 | 10,000.0.00 |
| LAG-3 | 0.0 | 0.0 | 0.0 | 0.0 | 0.0 | 47.2 | 11,111.1.00 |
| LDL R | 430.3 | 420.2 | 383.9 | 397.1 | 346.7 | 2.1 | 2,000.0.00 |
| Pepsinogen I | 19.7 | 2.5 | 8.8 | 5.0 | 0.0 | 0.5 | 740.7 |
| RANK | 0.0 | 0.0 | 0.0 | 0.0 | 0.0 | 30.2 | 33,333.3.00 |
| RBP4 | 815.6 | 913.7 | 921.0 | 1,170.2.00 | 915.8 | 2.2 | 20,000.0.00 |
| SOST | 0.0 | 0.0 | 0.0 | 0.0 | 0.0 | 1.1 | 4,444.4.00 |
| Syndecan-1 | 0.0 | 0.0 | 0.0 | 4.1 | 0.0 | 10.2 | 11,111.1.00 |
| TACI | 0.0 | 0.0 | 0.0 | 0.0 | 0.0 | 47.9 | 40,000.0.00 |
| TFPI | 0.0 | 2.0 | 0.0 | 624.2 | 0.0 | 43.5 | 100.000. |
| | | | | | | | 0 |
| TSP-1 | 80,544. 6.00 | 61,163. 6.00 | 57,557. 7.00 | 57,271. 1.00 | 80,035. 6.00 | 62.7 | 33,333.3.00 |
| TRAIL R2 | 0.0 | 0.0 | 0.0 | 0.0 | 0.0 | 4.7 | 4,000.0.00 |
| TRANCE | 0.0 | 0.0 | 0.0 | 0.0 | 0.0 | 249.1 | 40,000.0.00 |
| Troponin I | 0.0 | 0.0 | 0.0 | 0.0 | 0.0 | 34.9 | 66,666.7.00 |
| uPA | 4.4 | 2.9 | 9.3 | 44.4 | 4.1 | 4.2 | 1,333.3.00 |
| VE-Cadherin | 0.0 | 0.0 | 0.0 | 0.0 | 0.0 | 69.8 | 200,000. 0.00 |
| WISP-1 | 0.0 | 0.0 | 0.0 | 0.0 | 0.0 | 499.0 | 200,000. 0.00 |
| ANGPTL3 | 0.0 | 6.1 | 0.0 | 0.0 | 6.7 | 41.4 | 10,000.0.00 |
| bIG-H3 | 7,780.5.00 | 4,918.0.00 | 5,206.2.00 | 10,112. 4.00 | 6,686.6.00 | 117.7 | 10,000.0.00 |
| CA9 | 0.0 | 0.2 | 0.0 | 0.0 | 0.0 | 30.0 | 10,000.0.00 |
| Cathepsin B | 31.7 | 28.0 | 1,303.3.00 | 73.2 | 138.1 | 8.7 | 3,333.3.00 |
| CD23 | 0.0 | 0.0 | 0.0 | 0.0 | 0.0 | 19.8 | 10,000.0.00 |
| CHI3L1 | 0.0 | 8.1 | 0.0 | 0.0 | 0.0 | 28.6 | 10,000.0.00 |
| CTLA4 | 3.0 | 61.6 | 98.8 | 58.7 | 0.0 | 66.1 | 4,000.0.00 |
| Dkk-4 | 0.0 | 0.0 | 49.1 | 0.0 | 0.0 | 273.7 | 100,000. 0.00 |
| DPPIV | 2.2 | 0.0 | 0.0 | 22.7 | 9.9 | 34.0 | 22,222.2.00 |
| EDA-A2 | 0.0 | 152.7 | 0.0 | 0.0 | 133.1 | 124.1 | 10,000.0.00 |
| Epo R | 0.0 | 0.0 | 0.0 | 0.0 | 0.0 | 10.1 | 40,000.0.00 |
| FGF-6 | 0.0 | 0.0 | 0.0 | 0.0 | 0.0 | 103.6 | 3,333.3.00 |
| FGF-9 | 0.0 | 0.0 | 0.0 | 0.0 | 0.0 | 43.4 | 4,000.0.00 |
| Gas 1 | 2.9 | 22.4 | 0.0 | 0.0 | 25.2 | 15.9 | 3,703.7.00 |
| IGFBP-5 | 12.4 | 26.1 | 0.0 | 0.0 | 36.4 | 161.9 | 66,666.7.00 |
| IL-1 F5 | 0.0 | 0.0 | 0.0 | 0.0 | 0.0 | 58.8 | 7,407.4.00 |
| IL-1 F6 | 0.0 | 475.8 | 224.1 | 66.2 | 611.4 | 176.9 | 100,000. 0.00 |
| IL-1 F7 | 0.0 | 0.0 | 0.0 | 27.3 | 0.0 | 330.8 | 100,000. 0.00 |
| IL-1 F8 | 0.0 | 3.1 | 0.4 | 1.1 | 0.0 | 17.1 | 1,333.3.00 |
| IL-1 F9 | 505.1 | 205.0 | 0.0 | 7.6 | 415.9 | 385.8 | 100,000. 0.00 |
| IL-1 F10 | 1,720.3.00 | 730.4 | 2,479.7.00 | 1,675.1.00 | 1,003.2.00 | 1,890.9.00 | 200,000. 0.00 |
| IL-1 R5 | 3.6 | 8.3 | 7.9 | 1.6 | 0.0 | 9.2 | 1,000.0.00 |
| IL-17C | 0.0 | 0.0 | 0.0 | 0.0 | 0.0 | 39.2 | 14,814.8.00 |
| IL-18 | 0.0 | 0.0 | 0.0 | 0.0 | 37.3 | 60.0 | 40,000.0.00 |
| IL-20 | 0.0 | 21.8 | 0.0 | 0.0 | 0.0 | 31.8 | 11,111.1.00 |
| IL-34 | 7.6 | 8.2 | 23.4 | 0.5 | 1.7 | 7.6 | 13,333.3.00 |
| IL-5 Ra | 0.0 | 0.0 | 0.0 | 0.0 | 0.0 | 1,424.0.00 | 400,000. 0.00 |
| IL-10 Ra | 0.0 | 0.0 | 0.0 | 0.0 | 0.0 | 746.8 | 200,000. 0.00 |
| Layilin | 0.0 | 0.0 | 0.0 | 0.0 | 0.0 | 76.4 | 10,000.0.00 |
| Leptin R | 0.0 | 0.0 | 49.1 | 0.0 | 0.0 | 691.0 | 100,000. 0.00 |
| Marapsin | 0.0 | 0.0 | 0.0 | 0.0 | 0.0 | 41.1 | 6,666.7.00 |
| Mer | 0.0 | 27.3 | 2.8 | 0.0 | 0.0 | 37.8 | 10,000.0.00 |
| MMP-7 | 5.0 | 3.9 | 0.0 | 132.0 | 23.8 | 109.1 | 11,111.1.00 |
| P-Cadherin | 0.0 | 0.0 | 0.0 | 0.0 | 2.3 | 35.5 | 33,333.3.00 |
| Prostasin | 78.8 | 22.0 | 219.2 | 87.5 | 0.0 | 114.9 | 20,000.0.00 |
| PSMA | 1,276.5.00 | 0.0 | 1,230.6.00 | 0.0 | 0.0 | 2,590.7.00 | 100,000. 0.00 |
| SIGIRR | 8.6 | 19.2 | 216.3 | 0.0 | 0.0 | 970.0 | 100,000. 0.00 |
| TGFb RIII | 0.0 | 36.9 | 74.1 | 52.0 | 0.0 | 102.9 | 20,000.0.00 |
| TF | 14.4 | 0.0 | 11.2 | 0.0 | 0.0 | 5.3 | 4,000.0.00 |
| TWEAK | 0.0 | 0.0 | 0.0 | 0.0 | 0.0 | 60.5 | 100.000. |
| | | | | | | | 0 |
| ADAMTS13 | 0.0 | 0.0 | 270.8 | 0.0 | 0.0 | 205.1 | 100,000. 0.00 |
| Aggrecan | 0.0 | 0.0 | 0.0 | 0.0 | 0.0 | 43.4 | 20,000.0.00 |
| Angiotensinogen | 0.0 | 267.6 | 344.6 | 0.0 | 0.0 | 987.7 | 100,000. 0.00 |
| B7-H1 | 7.9 | 0.0 | 5.9 | 0.0 | 0.0 | 51.7 | 3,333.3.00 |
| BMPR-IA | 19.4 | 0.0 | 26.7 | 0.0 | 0.0 | 95.7 | 11,111.1.00 |
| BMPR-II | 0.0 | 0.0 | 0.0 | 0.0 | 0.0 | 175.1 | 33,333.3.00 |
| Cadherin-11 | 0.0 | 0.0 | 449.1 | 0.0 | 0.0 | 2,545.7.00 | 400,000. 0.00 |
| CD27 | 0.0 | 0.0 | 0.0 | 0.0 | 0.0 | 43.6 | 10,000.0.00 |
| CD6 | 136.5 | 803.5 | 991.2 | 0.0 | 378.9 | 562.3 | 100,000. 0.00 |
| Ck beta 8-1 | 0.0 | 0.0 | 0.0 | 0.0 | 0.0 | 54.3 | 3,703.7.00 |
| CNTF | 1,869.7.00 | 5,392.5.00 | 2,381.5.00 | 0.0 | 1,680.4.00 | 4,238.0.00 | 100,000. 0.00 |
| DNAM-1 | 11.3 | 80.6 | 187.6 | 0.0 | 0.0 | 482.3 | 100,000. 0.00 |
| EMMPRIN | 219.6 | 225.1 | 201.7 | 265.3 | 129.6 | 21.0 | 2,000.0.00 |
| FLRG | 4,427.3.00 | 4,234.0.00 | 7,193.2.00 | 4,646.3.00 | 4,625.7.00 | 58.9 | 10,000.0.00 |
| Follistatin-like 1 | 3,945.0.00 | 3,277.1.00 | 2,529.3.00 | 2,912.7.00 | 2,374.9.00 | 442.1 | 400,000. 0.00 |
| Fractalkine | 0.0 | 57.9 | 186.8 | 0.0 | 0.0 | 82.4 | 13,333.3.00 |
| Galectin-1 | 1,131.0.00 | 2,160.9.00 | 935.6 | 1,316.6.00 | 498.4 | 123.2 | 20,000.0.00 |
| GITR L | 0.0 | 1.4 | 0.0 | 0.0 | 0.0 | 13.8 | 200,000. 0.00 |
| Granulysin | 0.0 | 20.5 | 19.6 | 18.2 | 33.6 | 30.6 | 4,000.0.00 |
| IL-1 R3 | 0.0 | 0.0 | 76.1 | 0.0 | 0.0 | 72.6 | 10,000.0.00 |
| IL-15 R | 0.0 | 0.0 | 0.0 | 0.0 | 0.0 | 16.3 | 2,000.0.00 |
| IL-17E | 0.0 | 0.0 | 74.0 | 0.0 | 0.0 | 117.1 | 40,000.0.00 |
| IL-32 alpha | 0.0 | 0.0 | 0.0 | 0.0 | 0.0 | 2.9 | 1,333.3.00 |
| L1CAM-2 | 5.1 | 126.7 | 116.4 | 0.0 | 26.4 | 117.4 | 200.000. 0 |
| LRIG3 | 0.0 | 41.3 | 0.0 | 0.0 | 0.0 | 335.1 | 200.000. 0 |
| LRP-6 | 0.0 | 0.0 | 0.0 | 0.0 | 0.0 | 798.8 | 200.000. 0 |
| MEPE | 59.0 | 808.7 | 624.8 | 0.0 | 172.4 | 477.2 | 66,666.7.00 |
| Nectin-4 | 0.0 | 0.0 | 0.0 | 0.0 | 0.0 | 76.0 | 6,666.7.00 |
| Periostin | 3,848.4.00 | 4,386.5.00 | 3,045.3.00 | 2,142.5.00 | 3,844.2.00 | 187.9 | 7,407.4.00 |
| Persephin | 3,554.9.00 | 2,129.4.00 | 1,924.3.00 | 868.4 | 1,620.6.00 | 3,863.4.00 | 100.000. 0 |
| Renin | 0.0 | 0.0 | 0.0 | 0.0 | 0.0 | 28.1 | 10,000.0.00 |
| RGM-B | 7.0 | 0.0 | 14.6 | 0.0 | 0.0 | 67.3 | 100.000. 0 |
| ROBO3 | 3.4 | 2.8 | 1.1 | 0.0 | 0.0 | 3.0 | 2,000.0.00 |
| S100A8 | 0.0 | 0.0 | 0.0 | 0.0 | 0.0 | 61.3 | 10,000.0.00 |
| Siglec-7 | 0.0 | 0.0 | 2.5 | 0.0 | 0.0 | 11.5 | 2,000.0.00 |
| Syndecan-3 | 389.7 | 310.1 | 434.5 | 133.2 | 82.2 | 378.7 | 100.000. 0 |
| Thrombospondin -2 | 84.4 | 86.8 | 181.5 | 0.0 | 107.1 | 162.3 | 10,000.0.00 |
| Thrombospondin -5 | 7.4 | 0.0 | 0.0 | 0.0 | 0.0 | 5.1 | 3,333.3.00 |
| Tie-1 | 63.8 | 4.7 | 56.3 | 9.0 | 7.4 | 77.5 | 10,000.0.00 |
| ULBP-2 | 0.0 | 0.5 | 10.4 | 0.0 | 0.0 | 19.1 | 4,000.0.00 |
| ADAMS | 0.0 | 0.0 | 0.0 | 0.0 | 0.0 | 321.6 | 100.000. 0 |
| ADAM12 | 0.0 | 0.0 | 0.0 | 0.0 | 0.0 | 62.6 | 20,000.0.00 |
| B7-H3 | 0.0 | 0.0 | 0.0 | 0.0 | 0.0 | 5.7 | 4,000.0.00 |
| BMPR-IB | 0.0 | 0.0 | 0.0 | 0.0 | 0.0 | 11.2 | 3,333.3.00 |
| Cadherin-4 | 63.6 | 0.0 | 36.4 | 21.5 | 23.7 | 26.4 | 3,333.3.00 |
| Cadherin-13 | 0.0 | 0.0 | 0.0 | 0.0 | 0.0 | 41.8 | 11,111.1.00 |
| CD48 | 0.0 | 0.0 | 0.0 | 0.0 | 0.0 | 378.0 | 200.000. 0 |
| CD58 | 0.0 | 0.0 | 0.0 | 0.0 | 0.0 | 5.6 | 3,703.7.00 |
| CD84 | 0.0 | 0.0 | 0.0 | 0.0 | 30.4 | 381.4 | 100.000. 0 |
| CD99 | 621.8 | 440.4 | 324.2 | 624.8 | 364.0 | 3.7 | 1,333.3.00 |
| CD155 | 0.0 | 0.0 | 0.0 | 0.0 | 0.0 | 14.0 | 33,333.3.00 |
| CD229 | 0.0 | 0.0 | 0.0 | 0.0 | 0.0 | 24.3 | 10,000.0.00 |
| CEACAM-5 | 0.0 | 0.0 | 0.0 | 0.0 | 0.0 | 176.9 | 100.000. 0 |
| CF XIV | 182.8 | 44.6 | 105.5 | 87.2 | 131.9 | 33.8 | 20,000.0.00 |
| Cystatin A | 0.0 | 0.0 | 7.0 | 0.0 | 0.0 | 1.4 | 148.1 |
| Cystatin B | 73.3 | 120.8 | 33.4 | 128.2 | 20.8 | 6.2 | 1,333.3.00 |
| Cystatin E M | 0.8 | 2.3 | 17.2 | 1.2 | 0.6 | 0.8 | 370.4 |
| Desmoglein 2 | 5.7 | 0.0 | 13.4 | 3.9 | 7.4 | 13.4 | 6,666.7.00 |
| DR3 | 0.0 | 0.0 | 0.0 | 0.0 | 0.0 | 741.2 | 100.000. 0 |
| ErbB4 | 0.0 | 0.0 | 0.0 | 0.0 | 0.0 | 18.7 | 10,000.0.00 |
| ESAM | 0.0 | 0.0 | 0.0 | 0.0 | 0.0 | 4.0 | 3,333.3.00 |
| FGF-21 | 0.0 | 0.0 | 0.0 | 2.8 | 0.1 | 4.4 | 4,000.0.00 |
| Galectin-2 | 0.0 | 0.0 | 0.0 | 0.0 | 0.7 | 7.8 | 6,666.7.00 |
| Galectin-9 | 5.4 | 0.0 | 0.0 | 2.1 | 0.0 | 4.3 | 10,000.0.00 |
| ICOS | 39.0 | 0.0 | 0.0 | 43.0 | 96.9 | 101.4 | 100.000. 0 |
| JAM-A | 11.8 | 4.0 | 6.0 | 9.0 | 7.5 | 1.8 | 4,000.0.00 |
| JAM-B | 0.0 | 0.0 | 0.0 | 0.0 | 0.0 | 10.8 | 3,333.3.00 |
| Kallikrein 5 | 0.0 | 0.0 | 170.9 | 0.0 | 0.0 | 33.2 | 10,000.0.00 |
| Midkine | 0.0 | 1.3 | 0.0 | 0.0 | 0.0 | 3.5 | 2,222.2.00 |
| Pentraxin 3 | 1,922.1.00 | 1,127.6.00 | 1,210.4.00 | 1,948.8.00 | 1,644.8.00 | 6.4 | 3,333.3.00 |
| Pref-1 | 0.0 | 0.0 | 0.0 | 0.0 | 220.7 | 232.6 | 100.000. 0 |
| Siglec-10 | 0.0 | 0.0 | 0.0 | 0.0 | 0.0 | 298.9 | 200.000. 0 |
| SLAM | 0.0 | 0.0 | 0.0 | 0.0 | 0.0 | 52.0 | 33,333.3.00 |
| SP-D | 33.2 | 0.0 | 10.8 | 30.0 | 48.5 | 31.6 | 20,000.0.00 |
| Syndecan-4 | 0.9 | 0.4 | 0.6 | 1.1 | 0.3 | 0.0 | 37.0 |
| Testican 2 | 0.0 | 0.0 | 0.0 | 0.0 | 0.0 | 64.0 | 40,000.0.00 |
| TIM-3 | 0.0 | 0.0 | 0.0 | 4.1 | 10.4 | 7.1 | 10,000.0.00 |
| TLR4 | 0.0 | 0.0 | 0.0 | 0.0 | 0.0 | 10.6 | 7,407.4.00 |
| TRAIL | 2.0 | 3.0 | 2.9 | 1.7 | 4.6 | 2.1 | 666.7 |
| ULBP-1 | 0.0 | 0.0 | 0.0 | 0.0 | 18.3 | 49.0 | 6,666.7.00 |
| ALK-1 | 0.0 | 0.0 | 0.0 | 0.0 | 0.0 | 13.8 | 3,333.3.00 |
| B7-H2 | 4.1 | 5.1 | 13.6 | 0.0 | 0.0 | 9.3 | 2,000.0.00 |
| BLAME | 10.5 | 15.4 | 30.2 | 16.7 | 18.9 | 24.9 | 133,333.3.00 |
| BMP-8 | 0.0 | 76.3 | 148.4 | 81.3 | 123.9 | 102.5 | 40,000.0.00 |
| CD28 | 0.0 | 56.7 | 1.4 | 19.5 | 0.0 | 93.3 | 20,000.0.00 |
| Common beta Chain | 352.9 | 98.1 | 65.4 | 327.3 | 498.2 | 174.6 | 10,000.0.00 |
| Contactin-1 | 0.0 | 13.8 | 14.1 | 2.9 | 0.0 | 13.3 | 4,000.0.00 |
| Desmoglein-1 | 0.0 | 38.5 | 41.4 | 43.3 | 17.0 | 41.4 | 10,000.0.00 |
| Desmoglein-3 | 48.2 | 36.8 | 53.6 | 27.1 | 29.1 | 25.7 | 10,000.0.00 |
| EDAR | 13.5 | 10.6 | 24.5 | 11.7 | 0.0 | 30.3 | 4,000.0.00 |
| EphA1 | 0.0 | 73.9 | 48.1 | 0.0 | 0.0 | 29.1 | 10,000.0.00 |
| EphB6 | 11.0 | 57.5 | 57.3 | 26.6 | 3.4 | 21.4 | 4,000.0.00 |
| Ephrin-B3 | 552.4 | 665.3 | 686.0 | 0.0 | 0.0 | 401.0 | 100,000.0.00 |
| Epiregulin | 77.8 | 491.2 | 309.9 | 79.6 | 42.8 | 205.6 | 800,000.0.00 |
| FGF-12 | 115.9 | 125.6 | 190.4 | 165.8 | 99.5 | 99.7 | 2,000.0.00 |
| FGF-17 | 61.6 | 96.0 | 115.8 | 127.2 | 118.6 | 156.2 | 20,000.0.00 |
| FOLR2 | 0.0 | 4.9 | 1.2 | 3.1 | 0.0 | 5.7 | 20,000.0.00 |
| Galectin-8 | 4.5 | 3.2 | 8.2 | 3.2 | 0.0 | 11.2 | 1,000.0.00 |
| GHR | 8.0 | 1.6 | 6.5 | 13.0 | 0.0 | 14.5 | 4,000.0.00 |
| Glypican 1 | 226.8 | 315.8 | 286.9 | 437.2 | 112.6 | 41.9 | 10,000.0.00 |
| Glypican 5 | 0.0 | 110.0 | 167.4 | 130.7 | 0.0 | 164.5 | 10,000.0.00 |
| IFN-gamma R1 | 0.4 | 0.0 | 1.9 | 0.0 | 0.0 | 2.1 | 1,000.0.00 |
| IL-22 R alpha 1 | 8.8 | 12.4 | 10.8 | 11.6 | 0.0 | 9.2 | 4,000.0.00 |
| IL-22BP | 26.8 | 121.4 | 290.9 | 30.7 | 80.2 | 112.9 | 100,000.0.00 |
| IL-23 R | 0.0 | 1.8 | 0.8 | 0.0 | 0.0 | 12.9 | 4,000.0.00 |
| IL-31 RA | 0.0 | 68.5 | 8.2 | 2.4 | 0.0 | 26.8 | 10,000.0.00 |
| IL-7 R alpha | 74.1 | 101.6 | 199.7 | 102.9 | 52.7 | 14.4 | 2,000.0.00 |
| Integrin alpha 5 | 941.0 | 755.5 | 1,087.4.00 | 0.0 | 40.4 | 564.5 | 200,000.0.00 |
| MDM2 | 1.1 | 22.8 | 0.0 | 0.0 | 0.0 | 21.3 | 20,000.0.00 |
| Nectin-1 | 21.8 | 120.7 | 304.0 | 0.0 | 26.7 | 243.5 | 100,000.0.00 |
| NKp30 | 0.0 | 0.0 | 8.6 | 0.0 | 0.0 | 14.4 | 10,000.0.00 |
| Nogo Receptor | 74.2 | 80.4 | 168.3 | 0.0 | 0.9 | 111.4 | 40,000.0.00 |
| Notch-3 | 36.6 | 55.9 | 50.9 | 13.0 | 16.8 | 14.3 | 10,000.0.00 |
| OSM R beta | 514.8 | 762.4 | 974.3 | 484.3 | 299.9 | 286.4 | 100,000.0.00 |
| Prolactin R | 0.0 | 42.0 | 8.6 | 0.0 | 0.0 | 72.9 | 10,000.0.00 |
| RELT | 64.2 | 43.3 | 70.6 | 57.2 | 33.8 | 20.3 | 10,000.0.00 |
| Ryk | 0.0 | 4.1 | 27.4 | 0.0 | 0.0 | 62.2 | 10,000.0.00 |
| Semaphorin 6D | 0.0 | 105.4 | 55.9 | 51.8 | 0.0 | 124.0 | 100,000.0.00 |
| Semaphorin 7A | 4,160.3.00 | 189.1 | 500.7 | 150.7 | 1,801.9.00 | 41.0 | 20,000.0.00 |
| Siglec-11 | 0.0 | 918.3 | 852.0 | 382.1 | 0.0 | 563.9 | 200,000.0.00 |
| B7-2 | 8.2 | 1.1 | 0.0 | 0.0 | 0.0 | 24.0 | 2,000.0.00 |
| BAFF R | 0.0 | 6.0 | 0.0 | 12.7 | 0.0 | 29.1 | 1,111.1.00 |
| Calcitonin | 2,123.9.00 | 0.0 | 0.0 | 2,211.5.00 | 0.0 | 7,831.8.00 | 100,000.0.00 |
| Calsyntenin-1 | 0.0 | 51.8 | 40.7 | 25.9 | 0.0 | 46.3 | 13,333.3.00 |
| Cathepsin E | 0.7 | 0.0 | 0.0 | 0.0 | 0.0 | 181.6 | 100,000.0.00 |
| cIAP-2 | 5.6 | 10.3 | 14.8 | 7.1 | 4.9 | 8.4 | 66,666.7.00 |
| CF VII | 0.0 | 374.5 | 0.0 | 328.2 | 0.0 | 566.7 | 20,000.0.00 |
| cMASP3 | 494.2 | 213.7 | 546.0 | 642.7 | 390.1 | 403.2 | 100,000.0.00 |
| Endocan | 11.0 | 30.0 | 0.0 | 14.2 | 8.8 | 18.7 | 1,000.0.00 |
| EphA2 | 0.0 | 21.5 | 0.0 | 18.2 | 16.5 | 30.4 | 20,000.0.00 |
| EphB4 | 26.8 | 12.8 | 6.2 | 34.6 | 19.0 | 14.9 | 20,000.0.00 |
| Ephrin-A4 | 0.0 | 774.5 | 0.0 | 0.0 | 0.0 | 5,050.5.00 | 20,000.0.00 |
| FGF-23 | 180.4 | 272.4 | 153.6 | 104.1 | 221.5 | 128.0 | 10,000.0.00 |
| FGF-5 | 106.6 | 99.5 | 59.7 | 34.0 | 0.0 | 84.3 | 100,000.0.00 |
| Flt-3 | 11.2 | 6.5 | 1.6 | 13.5 | 2.2 | 6.5 | 3,333.3.00 |
| GLP-1 | 34.6 | 0.0 | 0.1 | 32.9 | 0.0 | 33.5 | 6,666.7.00 |
| Glypican 2 | 0.0 | 0.0 | 0.0 | 60.6 | 0.0 | 156.1 | 33,333.3.00 |
| GM-CSF Ra | 0.0 | 0.0 | 0.0 | 1,608.5.00 | 0.0 | 2,494.4.00 | 100,000.0.00 |
| GP73 | 1,297.9.00 | 596.4 | 572.5 | 859.6 | 480.5 | 134.5 | 10,000.0.00 |
| HTRA2 | 62.4 | 32.7 | 37.2 | 60.3 | 0.0 | 104.3 | 100,000.0.00 |
| IL-20 Ra | 3,524.7.00 | 634.4 | 243.5 | 2,977.1.00 | 1,208.9.00 | 2,046.2.00 | 100,000.0.00 |
| IL-4 Ra | 10.2 | 3.1 | 0.0 | 11.5 | 0.0 | 20.7 | 3,333.3.00 |
| JAM-C | 0.0 | 0.0 | 0.0 | 0.0 | 0.0 | 34.6 | 10,000.0.00 |
| LH | 0.0 | 0.0 | 0.0 | 0.0 | 0.0 | 310.3 | 10,000.0.00 |
| Matrilin-3 | 236.6 | 167.1 | 125.0 | 175.1 | 154.9 | 12.2 | 2,000.0.00 |
| MeprinA | 2,185.6.00 | 1,998.7.00 | 2,243.6.00 | 2,939.7.00 | 1,307.5.00 | 2,860.7.00 | 100,000.0.00 |
| MSP R | 0.0 | 23.3 | 0.0 | 0.0 | 0.0 | 57.7 | 10,000.0.00 |
| N-Cadherin | 0.0 | 0.0 | 0.0 | 122.5 | 0.0 | 127.9 | 100,000.0.00 |
| Neprilysin-2 | 319.7 | 0.0 | 0.0 | 0.0 | 0.0 | 503.8 | 100,000.0.00 |
| NKp44 | 0.0 | 34.2 | 16.7 | 0.0 | 0.0 | 66.2 | 2,000.0.00 |
| PAPP-A | 3,421.1.00 | 1,631.1.00 | 0.0 | 6,990.3.00 | 876.8 | 3,641.9.00 | 100,000.0.00 |
| Pepsinogen II | 57.2 | 0.0 | 0.0 | 25.9 | 15.5 | 29.6 | 3,333.3.00 |
| Presenilin 1 | 2.3 | 0.4 | 2.1 | 0.0 | 0.0 | 15.7 | 10,000.0.00 |
| PTH | 0.0 | 0.0 | 0.0 | 10.8 | 0.0 | 9.2 | 6,666.7.00 |
| PYY | 546.8 | 0.0 | 0.0 | 660.7 | 203.8 | 992.0 | 40,000.0.00 |
| SOX2 | 0.0 | 23.3 | 669.9 | 0.0 | 0.0 | 476.4 | 100,000.0.00 |
| TFF3 | 206.3 | 246.6 | 284.9 | 173.0 | 264.3 | 317.0 | 100,000.0.00 |
| TFPI-2 | 58.5 | 32.4 | 42.7 | 0.0 | 0.0 | 24.5 | 33,333.3.00 |
| TRACP | 106.1 | 0.0 | 0.0 | 0.0 | 0.0 | 140.6 | 100,000.0.00 |
| Ubiquitin+1 | 0.0 | 0.0 | 4.9 | 198.9 | 0.0 | 93.5 | 100,000.0.00 |
| ACE | 45.5 | 0.0 | 0.0 | 31.3 | 0.0 | 62.0 | 33,333.3.00 |
| Activin RIB | 0.0 | 0.0 | 0.0 | 530.1 | 0.0 | 109.9 | 20,000.0.00 |
| ADAM23 | 0.0 | 0.0 | 0.0 | 515.2 | 0.0 | 51.3 | 10,000.0.00 |
| Artemin | 17.7 | 0.0 | 0.0 | 0.0 | 0.0 | 1,008.7.00 | 10,000.0.00 |
| Cardiotrophin-1 | 0.0 | 0.0 | 0.0 | 2,967.8.00 | 0.0 | 187.3 | 40,000.0.00 |
| Cathepsin V | 26.8 | 0.0 | 0.0 | 1,890.4.00 | 7.6 | 80.9 | 10,000.0.00 |
| FABP1 | 118.3 | 274.2 | 0.0 | 0.0 | 0.0 | 1,169.1.00 | 400,000.0.00 |
| FGF-20 | 62.6 | 44.1 | 24.2 | 493.2 | 0.0 | 113.1 | 10,000.0.00 |
| GDF-8 | 0.0 | 0.0 | 116.2 | 932.6 | 23.7 | 526.7 | 100,000.0.00 |
| HAI-1 | 0.0 | 0.0 | 18.0 | 0.0 | 0.0 | 30.4 | 11,111.1.00 |
| IL-27 Ra | 68.7 | 63.6 | 66.7 | 95.7 | 0.0 | 99.6 | 10,000.0.00 |
| Insulin R | 541.8 | 0.0 | 579.6 | 639.4 | 0.0 | 230.5 | 40,000.0.00 |
| Kallikrein 7 | 0.0 | 0.0 | 2.9 | 0.0 | 0.0 | 6.8 | 1,333.3.00 |
| LIF R alpha | 0.0 | 0.0 | 0.0 | 0.0 | 0.0 | 8.6 | 10,000.0.00 |
| Lipocalin-1 | 0.0 | 0.0 | 37.3 | 4.3 | 0.0 | 3.8 | 1,000.0.00 |
| LTbR | 0.0 | 0.0 | 0.0 | 0.0 | 0.0 | 7.3 | 400.0 |
| Mesothelin | 2.4 | 0.0 | 14.0 | 0.0 | 0.0 | 5.1 | 4,000.0.00 |
| MFRP | 108.8 | 0.0 | 131.1 | 0.0 | 0.0 | 447.3 | 40,000.0.00 |
| Neuropilin-2 | 0.0 | 17.4 | 20.4 | 0.0 | 0.0 | 17.7 | 4,000.0.00 |
| Neurturin | 208.2 | 32.0 | 0.0 | 0.0 | 0.0 | 27.9 | 10,000.0.00 |
| Nidogen-2 | 4,364.4.00 | 5,604.0.00 | 3,905.9.00 | 8,465.5.00 | 1,689.6.00 | 32.2 | 6,666.7.00 |
| Olfactomedin-2 | 0.0 | 115.7 | 33.5 | 0.0 | 0.0 | 67.5 | 40,000.0.00 |
| p53 | 64.0 | 45.2 | 35.3 | 11.6 | 0.0 | 37.1 | 10,000.0.00 |
| PD-ECGF | 43.0 | 69.3 | 81.3 | 0.0 | 0.0 | 158.0 | 10,000.0.00 |
| PDGF-CC | 223.2 | 541.8 | 861.7 | 166.4 | 62.1 | 170.9 | 13,333.3.00 |
| Progranulin | 461.3 | 639.7 | 729.4 | 611.8 | 493.9 | 24.7 | 10,000.0.00 |
| Ret | 140.0 | 189.6 | 2,577.7.00 | 0.0 | 249.6 | 803.2 | 40,000.0.00 |
| ROBO4 | 25.6 | 60.7 | 27.4 | 0.0 | 0.0 | 41.4 | 4,000.0.00 |
| Semaphorin 6B | 275.3 | 383.4 | 158.5 | 0.0 | 0.0 | 433.4 | 10,000.0.00 |
| Serpin F1 | 45.6 | 219.7 | 0.0 | 0.0 | 0.0 | 169.4 | 10,000.0.00 |
| SREC-I | 0.0 | 0.0 | 38.7 | 0.0 | 0.0 | 16.5 | 4,000.0.00 |
| SREC-II | 93.3 | 293.5 | 262.4 | 0.0 | 0.0 | 278.9 | 20,000.0.00 |
| TLR1 | 1.4 | 0.0 | 1.5 | 0.0 | 0.0 | 7.1 | 4,000.0.00 |
| TLR3 | 12.7 | 13.3 | 21.7 | 19.2 | 20.4 | 3.0 | 111.1 |
| TPP1 | 261.2 | 281.9 | 284.1 | 181.8 | 110.6 | 56.5 | 10,000.0.00 |
| TREM-2 | 11.7 | 0.0 | 6.8 | 0.0 | 0.9 | 4.5 | 444.4 |
| TrkC | 24.1 | 70.4 | 45.2 | 76.9 | 5.6 | 15.8 | 4,000.0.00 |
| TROY | 0.0 | 104.3 | 300.8 | 0.0 | 0.0 | 351.5 | 13,333.3.00 |
| Uromodulin | 6.8 | 0.0 | 0.0 | 0.0 | 0.0 | 16.6 | 4,000.0.00 |
| XIAP | 0.0 | 30.9 | 19.4 | 0.0 | 0.0 | 39.5 | 10,000.0.00 |
| 4-1BB Ligand | 0.0 | 0.0 | 0.0 | 0.0 | 0.0 | 80.1 | 40,000.0.00 |
| Activin RIIB | 0.0 | 0.0 | 0.0 | 435.1 | 0.0 | 356.1 | 20,000.0.00 |
| Aminopeptidase P2 | 0.0 | 0.0 | 0.0 | 0.0 | 0.0 | 279.2 | 100,000.0.00 |
| BAMBI | 0.0 | 0.0 | 0.0 | 0.0 | 0.0 | 62.8 | 4,000.0.00 |
| BOC | 0.0 | 0.0 | 0.0 | 3.5 | 0.0 | 4.0 | 740.7 |
| Brevican | 6.4 | 0.0 | 0.0 | 10.6 | 9.1 | 1.1 | 37.0 |
| Carbonic Anhydrase XII | 0.0 | 0.0 | 0.0 | 0.0 | 0.0 | 596.1 | 100,000.0.00 |
| Carboxypeptidas e A2 | 0.0 | 0.0 | 0.0 | 118.0 | 0.0 | 17.9 | 4,000.0.00 |
| CD300c | 0.0 | 0.0 | 0.0 | 1.0 | 0.0 | 82.5 | 4,000.0.00 |
| CD320 | 0.0 | 0.0 | 0.0 | 126.2 | 0.0 | 270.5 | 10,000.0.00 |
| CDNF | 0.0 | 0.0 | 0.0 | 12.0 | 0.0 | 4.6 | 1,000.0.00 |
| CDO | 0.0 | 0.0 | 0.0 | 0.0 | 0.0 | 70.2 | 4,000.0.00 |
| CHST1 | 0.0 | 3.7 | 3.3 | 43.5 | 0.0 | 40.6 | 20,000.0.00 |
| CHST4 | 0.0 | 0.0 | 0.0 | 599.6 | 0.0 | 216.4 | 40,000.0.00 |
| CILP-1 | 0.0 | 0.0 | 0.0 | 18.7 | 0.0 | 64.6 | 4,000.0.00 |
| CNTF R alpha | 3.6 | 0.0 | 0.0 | 0.0 | 0.0 | 7.4 | 1,000.0.00 |
| CRIM1 | 540.2 | 99.4 | 362.5 | 649.6 | 220.2 | 58.0 | 1,000.0.00 |
| CRTAC1 | 0.0 | 0.0 | 0.0 | 0.0 | 0.0 | 495.8 | 20,000.0.00 |
| CXADR | 0.0 | 4.6 | 6.1 | 22.0 | 7.1 | 20.3 | 2,000.0.00 |
| Dopa Decarboxylase | 0.0 | 0.0 | 0.0 | 2.9 | 0.0 | 4.1 | 1,000.0.00 |
| DPPII | 40.6 | 29.8 | 0.0 | 470.8 | 0.6 | 40.8 | 10,000.0.00 |
| DSPG3 | 0.0 | 0.0 | 0.0 | 182.4 | 288.7 | 247.4 | 20,000.0.00 |
| EMR2 | 0.0 | 0.0 | 0.0 | 7.6 | 0.0 | 4.7 | 1,333.3.00 |
| FCAR | 313.5 | 299.2 | 100.0 | 85.5 | 0.0 | 634.9 | 40,000.0.00 |
| FCRL1 | 0.0 | 0.0 | 0.0 | 45.7 | 0.0 | 164.6 | 40,000.0.00 |
| FCRL2 | 0.0 | 0.0 | 0.0 | 21.9 | 0.0 | 14.5 | 13,333.3.00 |
| Gas6 | 0.0 | 4.8 | 0.0 | 17.2 | 0.0 | 11.4 | 4,000.0.00 |
| GPR56 | 0.0 | 0.0 | 0.0 | 0.7 | 3.6 | 10.0 | 10,000.0.00 |
| GPVI | 0.0 | 0.0 | 0.0 | 0.0 | 0.0 | 4.7 | 1,000.0.00 |
| Hepsin | 1.2 | 52.3 | 76.1 | 127.3 | 0.0 | 15.7 | 10,000.0.00 |
| ILT2 | 0.0 | 0.0 | 0.0 | 0.1 | 0.0 | 1.9 | 1,000.0.00 |
| Jagged 2 | 0.0 | 0.0 | 0.0 | 370.7 | 0.0 | 733.0 | 40,000.0.00 |
| Kirrel3 | 4.5 | 7.0 | 17.8 | 0.0 | 0.0 | 33.0 | 20,000.0.00 |
| KLF4 | 0.0 | 0.0 | 0.0 | 274.2 | 0.0 | 230.2 | 20,000.0.00 |
| LAIR1 | 0.0 | 0.0 | 5.6 | 6.3 | 0.0 | 90.4 | 100,000.0.00 |
| LAMP | 14.1 | 61.8 | 0.0 | 150.1 | 0.0 | 161.0 | 40,000.0.00 |
| LAMP1 | 0.0 | 0.0 | 0.0 | 178.5 | 0.0 | 77.2 | 40,000.0.00 |
| MDGA1 | 0.0 | 13.2 | 0.0 | 56.5 | 0.0 | 76.1 | 10,000.0.00 |
| MIS RII | 37.2 | 46.0 | 1.3 | 88.0 | 5.8 | 36.1 | 10,000.0.00 |
| Neurexin 3 beta | 0.0 | 0.0 | 0.0 | 5.4 | 3.6 | 40.0 | 4,000.0.00 |
| AMIGO | 91.1 | 223.4 | 236.0 | 1,257.4.00 | 473.2 | 1,062.5.00 | 100,000.0.00 |
| Aminopeptidase LRAP | 0.0 | 33,450.3.00 | 3,413.1.00 | 43,387.0.00 | 5,168.7.00 | 14,835.8.00 | 200,000.0.00 |
| Amnionless | 0.0 | 194.4 | 0.0 | 0.0 | 0.0 | 1,079.3.00 | 40,000.0.00 |
| Arylsulfatase A | 0.0 | 1,125.9.00 | 2.4 | 2,500.9.00 | 374.2 | 1,773.0.00 | 100,000.0.00 |
| Bcl-w | 0.0 | 0.0 | 0.0 | 2.7 | 0.0 | 0.8 | 1,000.0.00 |
| CD109 | 0.0 | 104.1 | 3,281.0.00 | 0.0 | 0.0 | 6,112.7.00 | 100,000.0.00 |
| CD157 | 0.5 | 0.7 | 0.2 | 2.0 | 0.0 | 0.5 | 1,000.0.00 |
| CD34 | 0.0 | 0.0 | 10.5 | 29.7 | 38.9 | 35.6 | 40,000.0.00 |
| CD83 | 0.0 | 0.0 | 0.0 | 0.0 | 0.0 | 104.4 | 10,000.0.00 |
| CLEC-1 | 0.0 | 5,629.2.00 | 0.0 | 10,448.0.00 | 0.0 | 6,931.7.00 | 200,000.0.00 |
| CLEC10A | 0.0 | 0.0 | 0.0 | 0.0 | 0.0 | 59.7 | 10,000.0.00 |
| CMG-2 | 21.6 | 49.8 | 3.0 | 137.2 | 52.5 | 103.5 | 40,000.0.00 |
| CREG | 0.0 | 0.0 | 0.0 | 7.8 | 0.0 | 118.6 | 100,000.0.00 |
| Cystatin SN | 0.0 | 0.0 | 0.0 | 0.0 | 3.8 | 47.2 | 11,111.1.00 |
| Cytokeratin-8 | 0.0 | 0.0 | 0.0 | 0.0 | 0.0 | 236.3 | 100,000.0.00 |
| Dectin-1 | 0.0 | 3.6 | 0.0 | 0.0 | 0.0 | 33.9 | 3,333.3.00 |
| Desmocollin-3 | 0.0 | 0.0 | 0.0 | 0.0 | 0.0 | 30.7 | 20,000.0.00 |
| Endoglycan | 21.9 | 232.0 | 365.6 | 0.0 | 258.6 | 196.0 | 200,000.0.00 |
| Galectin-4 | 0.0 | 0.0 | 0.0 | 0.0 | 0.0 | 7.1 | 1,000.0.00 |
| HAPLN1 | 61.8 | 0.0 | 3.1 | 5.8 | 0.0 | 194.1 | 200,000.0.00 |
| Jagged 1 | 0.0 | 34.4 | 0.0 | 0.0 | 0.0 | 540.2 | 100,000.0.00 |
| Langerin | 0.0 | 0.0 | 0.0 | 0.0 | 0.0 | 353.7 | 40,000.0.00 |
| Lumican | 1,491.5.00 | 1,139.3.00 | 1,367.0.00 | 1,337.3.00 | 955.4 | 20.2 | 3,333.3.00 |
| Matriptase | 451.2 | 0.0 | 0.0 | 338.6 | 182.1 | 131.8 | 100,000.0.00 |
| MEP1B | 0.0 | 0.0 | 0.0 | 279.7 | 71.0 | 295.2 | 13,333.3.00 |
| Nectin-3 | 0.0 | 0.0 | 0.0 | 0.0 | 0.0 | 17.3 | 13,333.3.00 |
| OX40 | 0.0 | 0.0 | 0.0 | 0.0 | 0.0 | 37.8 | 6,666.7.00 |
| OX40 Ligand | 67.7 | 0.0 | 0.0 | 0.0 | 0.0 | 222.3 | 40,000.0.00 |
| p27 | 0.0 | 0.0 | 0.0 | 0.0 | 0.0 | 87.6 | 20,000.0.00 |
| Pappalysin-2 | 0.0 | 0.0 | 0.0 | 0.0 | 0.0 | 78.3 | 20,000.0.00 |
| Plexin B3 | 0.0 | 0.0 | 0.0 | 0.0 | 1.9 | 16.1 | 2,222.2.00 |
| Plexin D1 | 0.0 | 0.0 | 0.0 | 0.0 | 0.0 | 4,597.2.00 | 200,000.0.00 |
| proGRP | 0.0 | 0.0 | 0.0 | 185.2 | 0.0 | 291.3 | 100,000.0.00 |
| PSA-total | 0.0 | 0.0 | 0.0 | 0.0 | 0.1 | 0.5 | 2,222.2.00 |
| Reg1B | 0.0 | 0.0 | 0.0 | 179.4 | 0.0 | 249.1 | 10,000.0.00 |
| RGM-A | 0.0 | 0.5 | 0.4 | 0.0 | 2.7 | 3.1 | 2,222.2.00 |
| ROBO2 | 0.0 | 0.0 | 0.0 | 60.3 | 0.0 | 552.3 | 10,000.0.00 |
| Spinesin | 147.4 | 311.6 | 456.6 | 79.5 | 569.8 | 194.7 | 33,333.3.00 |
| TWEAK R | 0.0 | 88.6 | 56.2 | 77.3 | 0.0 | 82.5 | 20,000.0.00 |
| ULBP-3 | 8.9 | 93.7 | 35.0 | 233.2 | 25.3 | 29.8 | 10,000.0.00 |
| Activin RIIA | 0.0 | 0.0 | 0.0 | 40.0 | 0.0 | 264.5 | 40,000.0.00 |
| Biglycan | 0.0 | 0.0 | 0.0 | 16.6 | 0.0 | 75.0 | 10,000.0.00 |
| CA13 | 0.0 | 0.0 | 8.9 | 50.9 | 15.0 | 28.3 | 10,000.0.00 |
| CA2 | 0.0 | 640.4 | 0.0 | 3,747.8.00 | 0.0 | 2,933.6.00 | 100,000.0.00 |
| CA72-4 | 0.0 | 0.0 | 0.0 | 10.2 | 0.0 | 73.3 | 10,000.0.00 |
| CLEC-2 | 0.0 | 0.0 | 0.0 | 49.0 | 0.0 | 723.1 | 100,000.0.00 |
| C-myc | 0.0 | 0.0 | 0.0 | 0.0 | 0.0 | 294.1 | 20,000.0.00 |
| Cystatin D | 0.0 | 0.0 | 4.5 | 16.6 | 0.0 | 58.5 | 4,000.0.00 |
| Erythropoietin | 0.0 | 0.0 | 0.0 | 1,397.6.00 | 0.0 | 2,494.8.00 | 40,000.0.00 |
| FCRL5 | 0.0 | 0.0 | 0.0 | 1,637.5.00 | 344.4 | 1,295.1.00 | 100,000.0.00 |
| FGF-16 | 0.0 | 0.0 | 0.7 | 12.5 | 0.0 | 214.4 | 10,000.0.00 |
| GATA-4 | 0.0 | 0.0 | 0.0 | 236.3 | 0.0 | 179.5 | 20,000.0.00 |
| GFR alpha-1 | 12.9 | 24.5 | 0.0 | 62.7 | 0.0 | 13.4 | 10,000.0.00 |
| GFR alpha-2 | 0.0 | 0.0 | 0.0 | 6.3 | 0.0 | 23.1 | 4,000.0.00 |
| Granzyme B | 0.0 | 0.0 | 32.9 | 273.2 | 62.4 | 195.4 | 10,000.0.00 |
| Granzyme H | 0.0 | 0.0 | 0.0 | 147.2 | 0.4 | 95.1 | 10,000.0.00 |
| HIF-1 alpha | 0.0 | 0.0 | 0.0 | 12.1 | 0.0 | 29.7 | 40,000.0.00 |
| htPAPP-A | 1,043.7.00 | 2,058.4.00 | 1,129.7.00 | 3,424.3.00 | 359.7 | 178.5 | 100,000.0.00 |
| IFNb | 0.0 | 0.0 | 0.0 | 831.8 | 0.0 | 308.9 | 100,000.0.00 |
| IL-17 RC | 0.0 | 92.7 | 0.0 | 163.9 | 56.6 | 285.9 | 10,000.0.00 |
| IL-19 | 0.0 | 0.0 | 0.0 | 35.5 | 0.0 | 39.1 | 3,703.7.00 |
| IL-20 R beta | 0.0 | 0.0 | 0.0 | 35.5 | 0.0 | 147.5 | 20,000.0.00 |
| IL-22 | 0.0 | 2.5 | 4.3 | 3.9 | 0.0 | 6.3 | 3,703.7.00 |
| ILT4 | 0.0 | 0.0 | 0.0 | 7.5 | 0.0 | 30.9 | 20,000.0.00 |
| LAIR2 | 0.0 | 0.0 | 0.0 | 1,563.4.00 | 0.0 | 1,047.4.00 | 100,000.0.00 |
| LSECtin | 37.3 | 253.7 | 0.0 | 1,027.1.00 | 296.4 | 264.2 | 20,000.0.00 |
| Netrin-4 | 0.0 | 0.0 | 0.0 | 638.8 | 0.0 | 565.2 | 200,000.0.00 |
| Norrin | 0.0 | 0.0 | 0.0 | 48.8 | 0.0 | 188.5 | 33,333.3.00 |
| NRG1-alpha | 0.0 | 0.0 | 0.0 | 1.2 | 0.0 | 0.6 | 1,333.3.00 |
| PD-L2 | 619.7 | 510.1 | 0.0 | 4,028.6.00 | 898.0 | 518.0 | 100,000.0.00 |
| PDX-1 | 0.0 | 0.0 | 0.0 | 318.6 | 0.0 | 195.1 | 10,000.0.00 |
| Podocalyxin | 1.4 | 0.0 | 0.0 | 13.0 | 1.7 | 22.1 | 10,000.0.00 |
| RGM-C | 0.0 | 0.0 | 0.0 | 285.5 | 0.0 | 238.1 | 3,703.7.00 |
| S100A1 | 0.0 | 0.0 | 0.0 | 1,882.9.00 | 0.0 | 1,519.3.00 | 33,333.3.00 |
| Semaphorin 6A | 39.0 | 0.0 | 0.0 | 1,247.2.00 | 108.0 | 239.8 | 20,000.0.00 |
| SLITRKS | 714.5 | 0.0 | 0.0 | 3,297.3.00 | 0.0 | 1,338.3.00 | 100,000.0.00 |
| SR-AI | 0.0 | 0.0 | 0.0 | 397.9 | 0.0 | 161.0 | 20,000.0.00 |
| ST6GAL1 | 0.0 | 0.0 | 0.0 | 440.9 | 12.1 | 131.6 | 11,111.1.00 |
| Thyroid Peroxidase | 438.3 | 0.0 | 42.9 | 985.8 | 0.0 | 894.8 | 100,000.0.00 |
| Troponin C | 309.5 | 0.0 | 0.0 | 898.6 | 159.8 | 398.1 | 33,333.3.00 |
| Activin R1A | 0.0 | 0.0 | 0.0 | 0.0 | 0.0 | 281.6 | 20,000.0.00 |
| ASAHL | 0.0 | 0.0 | 0.0 | 9.2 | 0.0 | 119.8 | 40,000.0.00 |
| B4GalT1 | 0.0 | 0.0 | 0.0 | 0.0 | 0.0 | 104.2 | 40,000.0.00 |
| BAI1 | 0.0 | 0.0 | 0.0 | 0.7 | 0.0 | 120.2 | 20,000.0.00 |
| Brorin | 0.0 | 0.0 | 0.0 | 0.0 | 0.0 | 29.6 | 6,666.7.00 |
| C1qTNF4 | 6.0 | 0.0 | 0.0 | 0.0 | 0.0 | 23.2 | 1,481.5.00 |
| CA14 | 0.0 | 0.0 | 0.0 | 0.3 | 0.0 | 3.4 | 2,000.0.00 |
| CA4 | 0.0 | 0.0 | 0.0 | 0.0 | 0.0 | 3.0 | 6,666.7.00 |
| CA6 | 0.0 | 0.0 | 0.0 | 0.0 | 0.0 | 35.2 | 20,000.0.00 |
| CA8 | 0.0 | 0.0 | 0.0 | 0.0 | 0.0 | 297.0 | 40,000.0.00 |
| Cadherin-6 | 0.0 | 0.0 | 4.0 | 0.0 | 0.0 | 5.1 | 4,000.0.00 |
| Caspr2 | 0.0 | 0.0 | 0.0 | 0.0 | 0.0 | 22.9 | 10,000.0.00 |
| CD27 Ligand | 0.0 | 33.8 | 60.8 | 269.0 | 0.0 | 71.7 | 10,000.0.00 |
| CD300a | 0.3 | 20.9 | 0.0 | 0.0 | 0.0 | 11.0 | 10,000.0.00 |
| CD300e | 163.1 | 0.0 | 0.0 | 0.0 | 0.0 | 1,636.5.00 | 200,000.0.00 |
| CD300f | 0.0 | 0.0 | 0.0 | 59.5 | 0.0 | 77.8 | 2,000.0.00 |
| CD4 | 0.0 | 0.0 | 0.0 | 0.0 | 0.0 | 26.9 | 7,407.4.00 |
| CD5 | 0.0 | 0.0 | 0.0 | 0.0 | 0.0 | 7.6 | 6,666.7.00 |
| CD69 | 0.0 | 0.0 | 0.0 | 10.5 | 6.8 | 8.2 | 400.0 |
| CK18 | 0.0 | 0.0 | 31.5 | 95.4 | 0.0 | 28.0 | 40,000.0.00 |
| CK19 | 0.0 | 0.0 | 0.0 | 0.0 | 0.0 | 35.7 | 40,000.0.00 |
| CPB1 | 0.0 | 0.0 | 0.0 | 0.5 | 0.0 | 1.5 | 4,444.4.00 |
| CRISP-2 | 0.0 | 8.3 | 13.2 | 18.1 | 0.0 | 18.2 | 10,000.0.00 |
| DDR1 | 0.0 | 0.0 | 0.0 | 0.0 | 0.0 | 111.0 | 40,000.0.00 |
| FUT8 | 0.0 | 0.0 | 0.0 | 62.0 | 0.0 | 72.4 | 10,000.0.00 |
| MIA | 1,953.3.00 | 46.0 | 4,889.1.00 | 135.4 | 0.0 | 3,502.7.00 | 100,000.0.00 |
| NTAL | 0.0 | 0.0 | 0.0 | 0.0 | 0.0 | 41.7 | 10,000.0.00 |
| NTB-A | 0.0 | 0.0 | 0.0 | 0.0 | 0.0 | 544.4 | 100,000.0.00 |
| OMgp | 0.0 | 0.0 | 0.0 | 2.2 | 15.8 | 259.3 | 10,000.0.00 |
| PEAR1 | 0.0 | 0.0 | 1.8 | 0.0 | 0.0 | 23.5 | 4,000.0.00 |
| Podoplanin | 0.0 | 0.0 | 0.0 | 14.3 | 4.6 | 15.3 | 10,000.0.00 |
| PTH1R | 0.0 | 0.0 | 0.0 | 0.0 | 0.0 | 4.0 | 10,000.0.00 |
| Reg4 | 0.0 | 0.0 | 0.0 | 8.1 | 0.0 | 21.4 | 13,333.3.00 |
| ROR1 | 0.0 | 0.0 | 0.0 | 294.5 | 0.0 | 123.7 | 33,333.3.00 |
| Semaphorin 4G | 0.0 | 0.0 | 0.0 | 0.0 | 0.0 | 13.7 | 2,000.0.00 |
| Serpin A5 | 0.0 | 0.0 | 0.0 | 22.6 | 0.0 | 30.8 | 13,333.3.00 |
| Serpin B6 | 90.2 | 316.5 | 0.0 | 466.2 | 0.0 | 85.4 | 10,000.0.00 |
| Siglec-1 | 11.9 | 0.0 | 0.0 | 8.2 | 0.0 | 23.5 | 4,000.0.00 |
| Sirtuin 2 | 3.6 | 32.3 | 0.0 | 0.0 | 0.0 | 8.9 | 111.1 |
| Sirtuin 5 | 0.0 | 0.0 | 53.6 | 279.7 | 95.9 | 228.8 | 40,000.0.00 |
| ANGPTL7 | 0.0 | 0.0 | 0.0 | 110.9 | 1.8 | 41.4 | 4,000.0.00 |
| CD36 | 0.0 | 102.5 | 0.0 | 0.0 | 0.0 | 265.9 | 20,000.0.00 |
| CLEC9a | 118.1 | 95.1 | 85.6 | 308.8 | 155.7 | 80.0 | 4,000.0.00 |
| CL-P1 | 111.2 | 0.0 | 0.0 | 523.0 | 426.0 | 150.4 | 40,000.0.00 |
| Dectin-2 | 14.2 | 14.7 | 13.0 | 5.9 | 0.4 | 5.5 | 4,000.0.00 |
| DLL4 | 120.4 | 73.0 | 0.0 | 171.3 | 0.0 | 167.6 | 100,000.0.00 |
| DSCAM | 459.3 | 77.3 | 0.0 | 667.2 | 859.5 | 265.5 | 10,000.0.00 |
| EDIL3 | 17.8 | 138.0 | 149.3 | 216.6 | 67.8 | 49.7 | 40,000.0.00 |
| ENPP-7 | 3.7 | 0.0 | 0.9 | 3.9 | 1.3 | 3.3 | 2,000.0.00 |
| Enteropeptidase | 0.0 | 0.0 | 0.0 | 2.8 | 0.0 | 13.7 | 2,000.0.00 |
| FCRL3 | 1.3 | 0.0 | 0.0 | 0.6 | 0.4 | 3.4 | 1,333.3.00 |
| FCRLB | 37.9 | 0.0 | 10.5 | 68.6 | 71.5 | 32.0 | 10,000.0.00 |
| FGF-3 | 64.2 | 0.0 | 0.0 | 387.1 | 0.0 | 995.7 | 100,000.0.00 |
| FLRT1 | 89.2 | 35.0 | 0.0 | 8.3 | 54.8 | 22.4 | 10,000.0.00 |
| FLRT2 | 28.7 | 0.0 | 0.0 | 111.4 | 41.4 | 59.9 | 3,333.3.00 |
| GBA3 | 652.6 | 0.0 | 0.0 | 865.5 | 254.5 | 488.9 | 20,000.0.00 |
| GDF-11 | 67.6 | 50.2 | 32.1 | 23.6 | 43.4 | 24.3 | 10,000.0.00 |
| Glycoprotein V | 14.3 | 62.7 | 25.1 | 74.7 | 65.7 | 28.8 | 10,000.0.00 |
| Granzyme A | 0.0 | 0.0 | 0.0 | 7.9 | 1.1 | 3.9 | 1,000.0.00 |
| IGSF4B | 34.9 | 46.5 | 3.9 | 29.4 | 29.0 | 19.9 | 3,333.3.00 |
| IL-28 R alpha | 11.9 | 11.4 | 0.0 | 22.1 | 18.4 | 8.5 | 1,000.0.00 |
| Kynureninase | 41.8 | 0.0 | 0.0 | 52.1 | 67.4 | 13.7 | 4,000.0.00 |
| LAMA4 | 26.3 | 145.6 | 113.4 | 137.9 | 22.0 | 26.1 | 10,000.0.00 |
| LRRC4 | 0.0 | 0.0 | 0.0 | 95.2 | 80.2 | 49.7 | 10,000.0.00 |
| LRRTM3 | 0.0 | 0.0 | 0.0 | 125.4 | 0.0 | 270.2 | 100,000.0.00 |
| NG2 | 0.0 | 18.5 | 54.9 | 6.3 | 30.3 | 23.0 | 20,000.0.00 |
| NQO-1 | 0.0 | 0.0 | 0.0 | 0.0 | 0.0 | 151.4 | 20,000.0.00 |
| PCSK2 | 0.0 | 0.0 | 0.0 | 0.0 | 0.0 | 516.7 | 40,000.0.00 |
| PILR-alpha | 0.5 | 3.7 | 1.1 | 0.0 | 0.0 | 1.1 | 400.0 |
| Plexin A4 | 0.0 | 0.0 | 0.0 | 37.0 | 26.5 | 26.3 | 10,000.0.00 |
| POGLUT1 | 200.2 | 438.5 | 281.3 | 758.2 | 198.7 | 346.4 | 100,000.0.00 |
| PRELP | 0.0 | 36.0 | 0.0 | 0.0 | 0.0 | 36.6 | 4,000.0.00 |
| Smad4 | 3.5 | 21.2 | 1.0 | 19.2 | 32.6 | 43.5 | 4,000.0.00 |
| SOX15 | 0.0 | 0.3 | 0.0 | 5.2 | 0.0 | 2.0 | 1,000.0.00 |
| SOX7 | 1,351.0.00 | 627.4 | 1,645.8.00 | 987.4 | 2,643.9.00 | 526.4 | 100,000.0.00 |
| SOX9 | 65.8 | 0.0 | 0.0 | 231.8 | 312.9 | 180.9 | 40,000.0.00 |
| Syntaxin 6 | 0.0 | 0.0 | 0.0 | 0.0 | 8.4 | 11.4 | 1,000.0.00 |
| TROP-2 | 0.0 | 3.0 | 0.0 | 0.0 | 0.8 | 2.9 | 400.0 |
| TSLP R | 182.7 | 0.0 | 0.0 | 198.7 | 163.7 | 407.8 | 20,000.0.00 |
| UNC5H4 | 14.4 | 0.0 | 0.0 | 58.3 | 5.0 | 12.0 | 1,000.0.00 |
| ADAM22 | 0.0 | 0.0 | 111.2 | 0.0 | 0.0 | 173.3 | 20,000.0.00 |
| ARSB | 42.0 | 96.5 | 188.1 | 189.1 | 0.0 | 150.5 | 10,000.0.00 |
| B3GNT2 | 0.0 | 0.0 | 142.3 | 92.2 | 0.0 | 170.7 | 20,000.0.00 |
| CA5B | 241.2 | 0.0 | 303.0 | 459.7 | 0.0 | 340.9 | 100,000.0.00 |
| Caspase 7 | 0.0 | 0.0 | 0.0 | 0.0 | 0.0 | 5,385.8.00 | 200,000.0.00 |
| Caspase 8 | 0.0 | 0.0 | 0.0 | 77.8 | 0.0 | 35.3 | 13,333.3.00 |
| CD11b | 427.0 | 83.4 | 425.5 | 709.6 | 0.0 | 315.0 | 40,000.0.00 |
| CD172g | 0.0 | 199.2 | 0.0 | 306.6 | 319.3 | 466.9 | 20,000.0.00 |
| CD39L2 | 0.0 | 0.0 | 0.0 | 0.0 | 0.0 | 626.9 | 20,000.0.00 |
| CD39L4 | 44.6 | 51.0 | 296.6 | 543.0 | 0.0 | 340.8 | 40,000.0.00 |
| CD49b | 0.0 | 0.0 | 0.0 | 37.4 | 393.6 | 45.2 | 11,111.1.00 |
| CD7 | 0.0 | 0.0 | 10.1 | 71.4 | 0.0 | 45.7 | 20,000.0.00 |
| CEACAM-3 | 438.3 | 521.5 | 371.0 | 1,158.7.00 | 6.6 | 221.9 | 20,000.0.00 |
| CPE | 20.5 | 0.0 | 64.6 | 62.2 | 272.4 | 17.6 | 10,000.0.00 |
| FABP6 | 6,272.7.00 | 4,254.3.00 | 4,027.0.00 | 6,021.1.00 | 5,659.1.00 | 2,169.2.00 | 40,000.0.00 |
| FAM3C | 250.1 | 154.0 | 1,001.7.00 | 1,681.9.00 | 1,474.9.00 | 1,341.0.00 | 3,703.7.00 |
| GDF-3 | 0.0 | 0.0 | 0.0 | 156.4 | 0.0 | 86.8 | 11,111.1.00 |
| GSTM1 | 0.0 | 0.0 | 773.0 | 44.2 | 0.0 | 1,276.8.00 | 200,000.0.00 |
| Kallikrein 11 | 0.0 | 0.0 | 0.0 | 0.1 | 0.1 | 0.2 | 133.3 |
| Kallikrein 12 | 0.3 | 0.5 | 9.3 | 4.7 | 0.0 | 8.5 | 148.1 |
| Kremen-2 | 57.4 | 0.0 | 332.9 | 527.8 | 162.2 | 224.6 | 4,000.0.00 |
| OSCAR | 743.0 | 1,685.2.00 | 412.9 | 3,873.9.00 | 59.6 | 21.7 | 66,666.7.00 |
| PTP1B | 0.0 | 0.0 | 215.6 | 371.9 | 0.0 | 204.8 | 20,000.0.00 |
| Reg3A | 0.0 | 0.0 | 0.0 | 14.2 | 0.0 | 2.7 | 148.1 |
| R-Spondin 2 | 0.0 | 0.0 | 0.0 | 0.1 | 0.0 | 4.1 | 4,000.0.00 |
| S100A13 | 0.0 | 0.0 | 0.0 | 0.0 | 0.0 | 0.0 | 3.7 |
| Semaphorin 4C | 502.2 | 112.6 | 322.9 | 856.0 | 0.0 | 676.9 | 20,000.0.00 |
| Sirtuin 1 | 0.0 | 16.0 | 77.9 | 151.3 | 0.0 | 76.9 | 100,000.0.00 |
| SMPD1 | 0.0 | 0.0 | 0.0 | 1,176.3.00 | 0.0 | 361.7 | 100,000.0.00 |
| Sortilin | 0.0 | 0.0 | 0.0 | 2,814.4.00 | 0.0 | 974.0 | 100,000.0.00 |
| SPINK1 | 0.0 | 0.0 | 0.0 | 0.9 | 0.0 | 65.7 | 11,111.1.00 |
| Stabilin-2 | 0.0 | 0.0 | 0.0 | 590.6 | 0.0 | 147.6 | 20,000.0.00 |
| SULT2A1 | 167.9 | 559.0 | 142.8 | 1,041.2.00 | 3.5 | 304.2 | 100,000.0.00 |
| TCN2 | 0.0 | 45.1 | 98.1 | 88.8 | 0.0 | 20.9 | 4,000.0.00 |
| THSD1 | 178.9 | 208.1 | 145.1 | 254.0 | 13.5 | 380.8 | 66,666.7.00 |
| TrkA | 0.0 | 0.0 | 105.1 | 348.6 | 0.0 | 172.7 | 200,000.0.00 |
| UCH-L3 | 0.0 | 106.6 | 276.5 | 335.1 | 0.0 | 168.9 | 200,000.0.00 |
| VAP-A | 47.6 | 0.0 | 0.0 | 375.2 | 0.0 | 213.2 | 20,000.0.00 |
| vWF-A2 | 3.9 | 3.0 | 10.9 | 18.4 | 0.0 | 11.4 | 1,333.3.00 |
| Wnt-4 | 204.8 | 73.8 | 251.1 | 309.5 | 0.0 | 316.6 | 40,000.0.00 |
| ADAMTSL-1 | 22,529.2.00 | 38,032.5.00 | 16,580.1.00 | 11,289.0.00 | 18,453.7.00 | 983.7 | 100,000.0.00 |
| AMSH | 193.4 | 152.1 | 2.2 | 242.2 | 213.0 | 102.0 | 10,000.0.00 |
| Annexin V | 1,463.9.00 | 1,555.1.00 | 788.0 | 956.1 | 1,134.4.00 | 623.0 | 100,000.0.00 |
| BATF3 | 0.0 | 0.0 | 0.0 | 0.0 | 0.0 | 13.4 | 33,333.3.00 |
| Bora | 642.3 | 48.1 | 376.3 | 165.5 | 631.0 | 266.9 | 100,000.0.00 |
| Cadherin-17 | 22.0 | 72.7 | 31.6 | 17.4 | 65.4 | 97.2 | 40,000.0.00 |
| Caveolin-2 | 29.1 | 24.4 | 14.2 | 1.9 | 26.2 | 21.1 | 10,000.0.00 |
| CD2 | 1,493.8.00 | 715.7 | 701.1 | 618.7 | 1,044.1.00 | 525.5 | 40,000.0.00 |
| CD200 R1 | 0.0 | 0.0 | 0.0 | 5.7 | 0.0 | 78.3 | 20,000.0.00 |
| CHST3 | 0.0 | 0.0 | 0.0 | 0.0 | 0.0 | 2,090.2.00 | 100,000.0.00 |
| COMT | 0.0 | 0.0 | 0.0 | 0.0 | 0.0 | 613.2 | 100,000.0.00 |
| Cystatin SA | 22.2 | 66.6 | 20.4 | 102.0 | 7.3 | 27.9 | 20,000.0.00 |
| DBH | 44.7 | 96.4 | 42.5 | 118.0 | 81.7 | 98.8 | 100,000.0.00 |
| Desmin | 73.7 | 0.0 | 180.8 | 28.7 | 67.0 | 66.6 | 20,000.0.00 |
| EXTL3 | 270.3 | 1,843.8.00 | 1,066.1.00 | 733.2 | 842.8 | 1,791.2.00 | 10,000.0.00 |
| Ficolin-1 | 0.0 | 30.7 | 0.0 | 27.9 | 0.0 | 40.0 | 10,000.0.00 |
| FosB | 39.9 | 32.1 | 56.5 | 4.2 | 0.0 | 67.1 | 10,000.0.00 |
| FRS2 | 71.8 | 0.0 | 0.0 | 83.2 | 0.0 | 106.0 | 20,000.0.00 |
| GATA-5 | 0.0 | 0.0 | 15.3 | 0.3 | 0.0 | 19.9 | 20,000.0.00 |
| GFAP | 93.8 | 0.0 | 0.0 | 0.0 | 109.8 | 126.1 | 200,000.0.00 |
| GLI-3 | 11.0 | 0.0 | 0.0 | 0.0 | 0.0 | 12.3 | 4,000.0.00 |
| HepaCAM | 0.4 | 0.0 | 0.1 | 0.0 | 0.0 | 1.8 | 2,000.0.00 |
| HIF-1 beta | 166.9 | 544.3 | 0.0 | 0.0 | 231.7 | 481.0 | 100,000.0.00 |
| HSD17B1 | 7.0 | 0.0 | 0.0 | 0.0 | 0.0 | 50.0 | 3,333.3.00 |
| IDO | 0.0 | 0.0 | 0.0 | 210.2 | 0.0 | 303.2 | 33,333.3.00 |
| Kallikrein 1 | 289.6 | 0.0 | 0.0 | 136.3 | 300.6 | 398.8 | 40,000.0.00 |
| Kell | 927.4 | 306.6 | 0.0 | 0.0 | 0.0 | 792.0 | 100,000.0.00 |
| MDL-1 | 0.0 | 0.1 | 5.6 | 4.7 | 3.3 | 3.2 | 740.7 |
| NPDC-1 | 0.0 | 0.0 | 0.0 | 0.0 | 0.3 | 0.3 | 200.0 |
| Numb | 15.0 | 39.0 | 3.5 | 29.7 | 2.9 | 14.5 | 4,000.0.00 |
| Olig2 | 2,104.8.00 | 2,542.3.00 | 377.1 | 2,786.9.00 | 1,576.4.00 | 943.6 | 100,000.0.00 |
| p63 | 34.7 | 19.4 | 0.0 | 165.8 | 133.6 | 131.4 | 40,000.0.00 |
| Pax3 | 0.0 | 0.0 | 0.0 | 0.0 | 0.0 | 39.2 | 100,000.0.00 |
| Semaphorin 4D | 765.1 | 250.5 | 0.0 | 613.8 | 191.0 | 350.7 | 11,111.1.00 |
| SPHK1 | 0.0 | 0.0 | 783.2 | 2,140.1.00 | 0.0 | 2,093.5.00 | 100,000.0.00 |
| TAZ | 0.0 | 0.0 | 15.9 | 62.2 | 0.0 | 45.7 | 20,000.0.00 |
| TC-PTP | 733.5 | 42.3 | 186.1 | 195.6 | 0.0 | 408.3 | 100,000.0.00 |
| TGM3 | 101.1 | 0.0 | 0.0 | 136.1 | 0.0 | 236.5 | 4,000.0.00 |
| TPST2 | 139.4 | 0.0 | 0.0 | 216.4 | 159.4 | 39.2 | 40,000.0.00 |
| TREML1 | 8.9 | 0.0 | 0.0 | 0.0 | 0.0 | 5.6 | 4,000.0.00 |
| Cf10 | 0.0 | 9.6 | 49.7 | 0.0 | 0.0 | 110.3 | 100,000.0.00 |
| CHMP2B | 0.0 | 0.0 | 0.0 | 0.0 | 0.0 | 1,466.1.00 | 100,000.0.00 |
| Contactin-3 | 0.0 | 0.0 | 0.0 | 0.0 | 0.0 | 52.8 | 100,000.0.00 |
| Cortactin | 0.0 | 76.8 | 309.9 | 0.0 | 0.0 | 117.8 | 40,000.0.00 |
| CrkL | 0.0 | 0.0 | 0.0 | 0.0 | 0.0 | 19.4 | 6,666.7.00 |
| Cyr61 | 185.1 | 316.3 | 213.9 | 333.3 | 176.4 | 21.7 | 4,000.0.00 |
| DAPP1 | 2,293.7.00 | 4,709.0.00 | 3,333.3.00 | 3,700.5.00 | 923.5 | 305.7 | 40,000.0.00 |
| DCTN1 | 38.5 | 375.3 | 72.0 | 557.0 | 461.1 | 201.8 | 100,000.0.00 |
| DFF45 | 0.0 | 36.8 | 2.7 | 2.8 | 0.0 | 20.9 | 10,000.0.00 |
| DRAK1 | 0.0 | 330.8 | 0.0 | 0.0 | 229.2 | 279.3 | 100,000.0.00 |
| GRAP2 | 76.1 | 305.6 | 598.1 | 289.9 | 298.0 | 138.1 | 40,000.0.00 |
| GRK5 | 0.0 | 0.0 | 0.0 | 0.0 | 717.9 | 200.6 | 100,000.0.00 |
| HAO-1 | 95.5 | 207.1 | 82.7 | 145.0 | 105.6 | 45.9 | 100,000.0.00 |
| LRIG1 | 16.0 | 30.5 | 10.4 | 35.2 | 25.2 | 13.2 | 40,000.0.00 |
| MMP-12 | 153.3 | 464.3 | 438.5 | 498.8 | 466.7 | 217.4 | 20,000.0.00 |
| NCK1 | 31.4 | 689.1 | 395.3 | 402.9 | 0.0 | 529.3 | 100,000.0.00 |
| Nectin-2 | 18.8 | 11.1 | 48.7 | 13.6 | 7.2 | 12.4 | 4,000.0.00 |
| Nesfatin-1 | 0.0 | 0.0 | 0.0 | 0.0 | 0.0 | 3.8 | 3,703.7.00 |
| Neurogranin | 2.6 | 10.7 | 0.0 | 8.1 | 6.5 | 4.1 | 6,666.7.00 |
| Nrf2 | 0.0 | 23.3 | 0.0 | 9.1 | 2.5 | 19.2 | 10,000.0.00 |
| NUDT5 | 0.0 | 58.9 | 106.6 | 78.5 | 33.2 | 56.1 | 11,111.1.00 |
| NUP85 | 6,996.0.00 | 10,981.1.00 | 5,527.3.00 | 9,827.4.00 | 6,479.7.00 | 366.9 | 100,000.0.00 |
| PAR1 | 76.7 | 150.4 | 348.5 | 0.0 | 95.4 | 261.4 | 100,000.0.00 |
| PP | 0.0 | 185.1 | 0.0 | 535.0 | 654.6 | 544.3 | 100,000.0.00 |
| PRX2 | 0.0 | 192.4 | 0.0 | 94.6 | 43.5 | 55.4 | 20,000.0.00 |
| PSMA1 | 102.4 | 202.2 | 224.0 | 178.9 | 134.7 | 109.0 | 20,000.0.00 |
| PU.1 | 0.0 | 4.3 | 0.0 | 17.9 | 3.6 | 7.7 | 10,000.0.00 |
| RalA | 0.0 | 181.0 | 0.0 | 66.4 | 0.0 | 51.1 | 40,000.0.00 |
| RCOR1 | 3.2 | 2.2 | 8.7 | 1.0 | 0.0 | 3.6 | 10,000.0.00 |
| Serpin B8 | 27.3 | 19.2 | 6.1 | 56.2 | 22.6 | 12.6 | 40,000.0.00 |
| SH2DIA | 0.0 | 12.6 | 0.0 | 9.8 | 0.0 | 9.2 | 4,000.0.00 |
| SHP-1 | 76.7 | 239.2 | 241.2 | 39.6 | 353.2 | 72.3 | 11,111.1.00 |
| Siglec-6 | 46.3 | 97.3 | 58.2 | 92.4 | 107.5 | 27.2 | 10,000.0.00 |
| SorCS3 | 0.0 | 0.0 | 30.4 | 0.0 | 0.0 | 102.3 | 100,000.0.00 |
| THAP11 | 0.0 | 246.1 | 113.2 | 201.8 | 487.7 | 127.6 | 100,000.0.00 |
| ULBP-4 | 892.0 | 698.1 | 1,012.5.00 | 1,116.2.00 | 0.0 | 307.6 | 100,000.0.00 |
| UNC5H3 | 59.6 | 237.5 | 226.4 | 279.3 | 238.2 | 46.8 | 10,000.0.00 |
| VAMP-1 | 0.0 | 0.0 | 0.0 | 0.0 | 0.0 | 1.5 | 1,481.5.00 |
| VAMP-2 | 0.0 | 6.7 | 152.5 | 67.5 | 0.0 | 106.6 | 100,000.0.00 |
| Visfatin | 306.7 | 611.9 | 57.6 | 486.6 | 297.0 | 86.5 | 40,000.0.00 |
| C1qTNF9 | 0.0 | 0.0 | 0.0 | 0.0 | 0.0 | 56.6 | 10,000.0.00 |
| CA5A | 0.0 | 0.0 | 0.0 | 0.0 | 0.0 | 242.8 | 3,333.3.00 |
| CANT1 | 21.5 | 87.7 | 48.4 | 154.7 | 162.4 | 37.4 | 11,111.1.00 |
| Cathepsin H | 0.0 | 0.0 | 0.0 | 0.0 | 0.0 | 73.9 | 20,000.0.00 |
| Contactin-5 | 0.0 | 0.0 | 0.0 | 0.0 | 0.0 | 47.6 | 10,000.0.00 |
| CTRC | 0.0 | 0.0 | 0.0 | 0.0 | 0.0 | 9.7 | 4,000.0.00 |
| Draxin | 0.0 | 0.0 | 0.0 | 0.0 | 0.0 | 14.4 | 1,000.0.00 |
| EphB2 | 0.0 | 0.0 | 0.0 | 0.0 | 0.0 | 1,467.2.00 | 100,000.0.00 |
| EphB3 | 0.0 | 0.0 | 0.0 | 0.0 | 0.0 | 87.5 | 10,000.0.00 |
| FABPS | 0.0 | 0.0 | 0.0 | 0.0 | 0.0 | 7.0 | 3,703.7.00 |
| Fgr | 0.0 | 0.0 | 0.0 | 0.0 | 0.0 | 80.1 | 100,000.0.00 |
| FKBP51 | 4.4 | 0.0 | 0.0 | 32.8 | 0.0 | 100.1 | 100,000.0.00 |
| FUCA1 | 0.0 | 0.0 | 0.0 | 0.0 | 0.0 | 71.5 | 40,000.0.00 |
| Galanin | 0.0 | 0.0 | 0.0 | 0.0 | 0.0 | 297.3 | 100,000.0.00 |
| GALNT10 | 0.0 | 0.0 | 0.0 | 47.1 | 0.0 | 334.1 | 40,000.0.00 |
| GKN1 | 0.0 | 0.0 | 0.0 | 0.0 | 0.0 | 751.1 | 100,000.0.00 |
| Glyoxalase II | 111.2 | 0.0 | 0.0 | 131.8 | 0.0 | 308.9 | 100,000.0.00 |
| HS3ST1 | 16.7 | 0.0 | 0.0 | 3.3 | 0.0 | 27.7 | 10,000.0.00 |
| HS3ST3B1 | 0.0 | 0.0 | 0.0 | 53.1 | 4.3 | 39.3 | 10,000.0.00 |
| Lin28 | 0.0 | 0.0 | 0.0 | 14.2 | 3.6 | 23.1 | 4,000.0.00 |
| LOXL2 | 1,054.1.00 | 4,022.4.00 | 0.0 | 873.4 | 62.8 | 396.1 | 100,000.0.00 |
| LRRTM4 | 0.0 | 0.0 | 0.0 | 0.0 | 0.0 | 17.7 | 2,000.0.00 |
| MAP1D | 0.0 | 0.0 | 0.0 | 0.0 | 0.0 | 7.5 | 10,000.0.00 |
| Matrilin-2 | 76.6 | 217.8 | 79.2 | 137.9 | 0.0 | 74.5 | 4,000.0.00 |
| MCEMP1 | 0.0 | 0.0 | 0.0 | 0.0 | 0.0 | 37.0 | 10,000.0.00 |
| Mcl-1 | 0.0 | 56.2 | 0.0 | 42.3 | 0.0 | 39.3 | 10,000.0.00 |
| MDGA2 | 0.0 | 0.0 | 0.0 | 87.9 | 0.0 | 499.2 | 66,666.7.00 |
| MEF2C | 0.0 | 0.0 | 0.0 | 1.4 | 0.0 | 2.6 | 2,000.0.00 |
| METAP2 | 1.5 | 2.8 | 0.0 | 0.6 | 0.0 | 2.3 | 2,222.2.00 |
| Neurocan | 0.0 | 0.0 | 0.0 | 0.0 | 0.0 | 1.1 | 2,000.0.00 |
| Nogo-A | 0.0 | 0.0 | 0.0 | 1.3 | 0.0 | 4.1 | 1,000.0.00 |
| PCK1 | 0.0 | 0.0 | 195.4 | 386.8 | 0.0 | 150.8 | 20,000.0.00 |
| PIGF-2 | 0.0 | 0.0 | 0.0 | 1.9 | 0.5 | 3.0 | 400.0 |
| PON1 | 193.5 | 0.0 | 0.0 | 152.7 | 196.0 | 266.0 | 100,000.0.00 |
| SALM4 | 0.0 | 0.0 | 0.0 | 52.3 | 0.0 | 64.6 | 10,000.0.00 |
| Semaphorin 6C | 0.0 | 0.0 | 0.0 | 493.1 | 0.0 | 2,797.8.00 | 40,000.0.00 |
| SorCS2 | 0.0 | 0.0 | 0.0 | 11.5 | 0.0 | 24.5 | 4,000.0.00 |
| ST3GAL1 | 470.6 | 1,644.0.00 | 3,583.5.00 | 9,858.4.00 | 0.0 | 2,968.3.00 | 100,000.0.00 |
| ST8SIA1 | 3.1 | 0.0 | 0.0 | 14.3 | 0.0 | 62.6 | 10,000.0.00 |
| TSK | 678.1 | 1,782.3.00 | 326.2 | 609.3 | 112.8 | 184.9 | 33,333.3.00 |
| ADA | 242.5 | 264.3 | 269.4 | 268.6 | 160.4 | 89.6 | 11,111.1.00 |
| AIF | 62.9 | 67.7 | 415.3 | 109.4 | 0.0 | 547.4 | 13,333.3.00 |
| AKR1C4 | 4,920.1.00 | 5,623.3.00 | 7,001.1.00 | 5,459.7.00 | 4,220.5.00 | 2,502.5.00 | 100,000.0.00 |
| ASAH2 | 350.5 | 392.6 | 471.4 | 469.9 | 371.0 | 173.0 | 33,333.3.00 |
| BCL-2 | 1,247.5.00 | 55.7 | 68.1 | 1,034.1.00 | 926.0 | 391.5 | 33,333.3.00 |
| BID | 1,173.6.00 | 1,101.0.00 | 1,198.4.00 | 1,120.0.00 | 372.1 | 754.3 | 33,333.3.00 |
| Calreticulin | 341.5 | 108.5 | 319.4 | 235.6 | 120.8 | 143.6 | 40,000.0.00 |
| Calreticulin-2 | 16.0 | 53.1 | 66.2 | 0.0 | 0.0 | 51.1 | 33,333.3.00 |
| CD314 | 882.0 | 1,508.1.00 | 142.1 | 1,000.0.00 | 1,171.3.00 | 948.7 | 100,000.0.00 |
| CD39L3 | 27.8 | 23.7 | 14.7 | 18.4 | 0.0 | 18.5 | 13,333.3.00 |
| CD51 | 239.0 | 386.6 | 270.0 | 126.8 | 170.8 | 239.9 | 40,000.0.00 |
| CD99-L2 | 148.8 | 108.6 | 137.3 | 94.4 | 78.5 | 59.4 | 20,000.0.00 |
| CDC25B | 16.1 | 314.8 | 303.4 | 161.0 | 196.1 | 258.7 | 33,333.3.00 |
| Cerberus 1 | 34.3 | 58.6 | 53.8 | 22.0 | 30.5 | 22.3 | 10,000.0.00 |
| CHST2 | 24.9 | 142.0 | 118.0 | 54.8 | 51.3 | 152.1 | 13,333.3.00 |
| Cochlin | 25.7 | 15.5 | 64.5 | 110.8 | 23.2 | 81.8 | 11,111.1.00 |
| CRELD2 | 58.6 | 61.1 | 39.4 | 82.5 | 61.0 | 46.9 | 13,333.3.00 |
| DC-SIGNR | 39.3 | 434.1 | 123.8 | 0.0 | 0.0 | 414.8 | 100,000.0.00 |
| eNOS | 554.9 | 643.0 | 397.5 | 631.4 | 387.9 | 356.0 | 100,000.0.00 |
| ENPP-2 | 205.9 | 712.2 | 470.0 | 223.4 | 544.4 | 219.2 | 33,333.3.00 |
| FABP4 | 54.8 | 13.1 | 37.8 | 24.2 | 1.4 | 15.1 | 3,703.7.00 |
| FcERI | 172.0 | 155.3 | 193.7 | 206.4 | 187.7 | 85.1 | 2,000.0.00 |
| FGF R5 | 0.0 | 13.5 | 0.0 | 0.0 | 0.0 | 76.7 | 2,000.0.00 |
| GALNT2 | 396.0 | 788.8 | 626.3 | 944.6 | 307.1 | 150.2 | 100,000.0.00 |
| GALNT3 | 0.0 | 41.6 | 0.0 | 0.0 | 51.7 | 131.3 | 100,000.0.00 |
| GIF | 0.0 | 5.8 | 4.3 | 0.0 | 1.5 | 8.3 | 11,111.1.00 |
| GPR111 | 0.0 | 35.4 | 44.3 | 0.0 | 0.0 | 85.8 | 20,000.0.00 |
| GUSB | 0.0 | 0.0 | 0.0 | 0.0 | 0.0 | 68.7 | 20,000.0.00 |
| Inhibin A | 59.0 | 187.0 | 213.6 | 0.0 | 0.0 | 168.8 | 40,000.0.00 |
| LILRB4 | 0.0 | 0.0 | 376.7 | 26.0 | 54.6 | 735.9 | 40,000.0.00 |
| Neuroglycan C | 92.4 | 197.2 | 301.0 | 319.9 | 255.9 | 281.0 | 33,333.3.00 |
| NKp46 | 6.1 | 7.7 | 2.7 | 0.6 | 0.0 | 7.2 | 2,000.0.00 |
| NPTXR | 58.7 | 93.8 | 67.1 | 142.3 | 89.5 | 73.6 | 13,333.3.00 |
| ROR2 | 0.0 | 21.4 | 21.9 | 28.5 | 11.3 | 24.5 | 2,000.0.00 |
| SCCA2 | 0.0 | 3,300.4.00 | 0.0 | 27.0 | 0.0 | 485.5 | 40,000.0.00 |
| Siglec-2 | 162.6 | 294.1 | 307.6 | 256.3 | 144.1 | 86.4 | 10,000.0.00 |
| SIRP alpha | 0.8 | 0.6 | 1.3 | 1.0 | 0.0 | 2.2 | 333.3 |
| SorCS1 | 42.0 | 502.6 | 298.2 | 184.9 | 361.0 | 265.9 | 100,000.0.00 |
| Trypsin 1 | 6,946.9.00 | 0.0 | 0.0 | 420.2 | 6,643.4.00 | 13,052.4.00 | 100,000.0.00 |
| Trypsin 3 | 2.6 | 3.1 | 2.2 | 9.1 | 0.0 | 3.8 | 2,000.0.00 |
| AMIGO2 | 1,180.1.00 | 299.9 | 408.6 | 842.4 | 786.3 | 192.1 | 20,000.0.00 |
| Arginase 1 | 42.3 | 0.0 | 0.0 | 0.0 | 0.0 | 105.9 | 10,000.0.00 |
| B7-H4 | 51.5 | 27.3 | 4.0 | 0.0 | 20.8 | 24.4 | 33,333.3.00 |
| Bcl-10 | 2,888.8.00 | 3,002.6.00 | 2,832.1.00 | 876.1 | 3,026.3.00 | 838.6 | 100,000.0.00 |
| CD42b | 616.9 | 532.4 | 468.8 | 217.2 | 339.7 | 105.8 | 100,000.0.00 |
| CD73 | 192.3 | 309.7 | 209.0 | 344.7 | 234.9 | 86.9 | 10,000.0.00 |
| CES1 | 133.2 | 166.3 | 208.6 | 199.5 | 259.3 | 34.6 | 2,000.0.00 |
| CES2 | 0.0 | 0.0 | 0.0 | 0.0 | 0.0 | 47.3 | 16,666.7.00 |
| cIAP-1 | 737.1 | 831.2 | 953.2 | 231.2 | 1,012.3.00 | 121.8 | 100,000.0.00 |
| Cyclophilin A | 0.0 | 70.6 | 57.8 | 0.0 | 30.8 | 63.1 | 10,000.0.00 |
| Cystatin S | 29.4 | 10.2 | 23.6 | 20.0 | 35.4 | 7.8 | 400.0 |
| DNMT3A | 372.2 | 2,560.2.00 | 1,118.0.00 | 480.7 | 1,298.5.00 | 561.8 | 20,000.0.00 |
| Epimorphin | 0.0 | 0.0 | 0.0 | 0.0 | 0.0 | 14.3 | 4,000.0.00 |
| GDF-9 | 0.0 | 0.0 | 0.0 | 54.4 | 0.0 | 1,165.9.00 | 40,000.0.00 |
| Glypican 3 | 0.0 | 0.0 | 0.0 | 75.8 | 39.0 | 101.1 | 33,333.3.00 |
| GPR115 | 428.6 | 0.0 | 0.0 | 1,051.7.00 | 494.3 | 681.4 | 100,000.0.00 |
| HE4 | 17.8 | 27.7 | 34.0 | 0.0 | 0.0 | 38.2 | 1,000.0.00 |
| HO-1 | 0.2 | 0.1 | 0.2 | 0.1 | 0.2 | 0.1 | 33.3 |
| HS3ST4 | 917.5 | 938.9 | 0.0 | 3,535.3.00 | 2,318.2.00 | 4,873.6.00 | 100,000.0.00 |
| IGSF3 | 20.3 | 15.9 | 25.2 | 22.6 | 0.0 | 41.4 | 20,000.0.00 |
| IL-17 RD | 66.5 | 0.0 | 0.0 | 171.8 | 45.2 | 94.8 | 13,333.3.00 |
| Integrin alpha 1 | 274.0 | 0.0 | 811.8 | 161.8 | 923.2 | 459.7 | 30,000.0.00 |
| KIR2DL3 | 1,424.0.00 | 527.6 | 1,202.3.00 | 0.0 | 1,637.8.00 | 448.6 | 40,000.0.00 |
| LAMP2 | 281.6 | 0.0 | 0.0 | 629.7 | 135.4 | 240.1 | 33,333.3.00 |
| LEDGF | 0.0 | 132.2 | 175.4 | 0.0 | 0.0 | 206.0 | 40,000.0.00 |
| MOG | 0.0 | 0.0 | 0.0 | 0.0 | 0.0 | 4.3 | 4,000.0.00 |
| Nestin | 34.3 | 0.0 | 5.8 | 20.0 | 18.0 | 23.8 | 20,000.0.00 |
| Neudesin | 0.0 | 0.0 | 0.0 | 0.0 | 2.9 | 33.7 | 3,333.3.00 |
| Neuroligin 2 | 285.1 | 0.0 | 0.0 | 0.0 | 340.0 | 108.1 | 20,000.0.00 |
| NKp80 | 99.6 | 0.0 | 0.0 | 124.5 | 103.0 | 70.5 | 4,000.0.00 |
| Osteoadherin | 12.3 | 0.0 | 0.0 | 19.8 | 19.7 | 12.3 | 2,000.0.00 |
| PDGF R alpha | 170.7 | 255.9 | 170.5 | 1.8 | 0.0 | 233.3 | 100,000.0.00 |
| PRDX4 | 1,839.1.00 | 151.6 | 314.2 | 0.0 | 498.4 | 1,138.0.00 | 100,000.0.00 |
| Syntaxin 4 | 0.4 | 9.4 | 0.0 | 6.0 | 5.2 | 11.9 | 4,444.4.00 |
| TAFA1 | 95.2 | 62.8 | 102.9 | 56.8 | 79.2 | 66.8 | 100,000.0.00 |
| TAFA2 | 12.4 | 27.4 | 0.0 | 57.4 | 0.0 | 60.4 | 100,000.0.00 |
| TAFA5 | 0.0 | 0.0 | 6.8 | 60.4 | 46.5 | 33.3 | 20,000.0.00 |
| Tenascin R | 114.6 | 130.1 | 8.4 | 0.0 | 61.0 | 77.3 | 100,000.0.00 |
| TGM4 | 566.6 | 1,426.8.00 | 1,212.1.00 | 1,425.3.00 | 580.5 | 751.6 | 100,000.0.00 |
| TMEFF1 | 62.7 | 0.0 | 0.0 | 118.4 | 127.4 | 104.3 | 20,000.0.00 |
| BLC | 0.0 | 0.0 | 0.0 | 0.0 | 0.1 | 0.2 | 74.1 |
| Eotaxin | 0.9 | 0.5 | 0.1 | 0.9 | 0.8 | 0.3 | 148.1 |
| Eotaxin-2 | 0.7 | 0.0 | 0.6 | 2.1 | 2.0 | 0.6 | 333.3 |
| G-CSF | 0.0 | 0.9 | 0.0 | 0.7 | 1.2 | 1.5 | 740.7 |
| GM-CSF | 0.6 | 0.0 | 0.0 | 0.0 | 0.5 | 1.6 | 1,000.0.00 |
| I-309 | 2.5 | 0.0 | 0.0 | 2.5 | 1.7 | 1.3 | 1,333.3.00 |
| ICAM-1 | 5.2 | 9.4 | 10.3 | 62.1 | 0.0 | 3.8 | 3,703.7.00 |
| IFNγ | 0.9 | 0.1 | 0.0 | 0.7 | 1.0 | 1.2 | 666.7 |
| IL-1a | 0.2 | 2.7 | 0.8 | 1.9 | 2.8 | 3.1 | 666.7 |
| IL-1b | 0.0 | 0.5 | 0.0 | 0.0 | 1.3 | 0.7 | 333.3 |
| IL-1Rα | 64.6 | 31.0 | 58.6 | 55.5 | 96.6 | 29.5 | 2,000.0.00 |
| IL-2 | 7.4 | 0.0 | 5.9 | 13.9 | 15.3 | 17.5 | 2,000.0.00 |
| IL-4 | 1.4 | 1.3 | 0.0 | 0.0 | 1.2 | 1.6 | 74.1 |
| IL-5 | 32.0 | 0.0 | 0.0 | 20.3 | 13.5 | 20.7 | 4,000.0.00 |
| IL-6 | 132.7 | 88.9 | 75.8 | 552.8 | 25.0 | 8.2 | 2,000.0.00 |
| IL-6R | 14.3 | 11.4 | 12.6 | 13.7 | 10.4 | 13.8 | 3,333.3.00 |
| IL-7 | 0.6 | 0.9 | 0.0 | 2.3 | 2.2 | 1.6 | 4,000.0.00 |
| IL-8 | 4.8 | 0.5 | 0.1 | 5.3 | 1.9 | 1.4 | 500.0 |
| IL-10 | 0.0 | 0.0 | 0.0 | 0.1 | 0.0 | 0.4 | 1,333.3.00 |
| IL-11 | 9.2 | 2.9 | 1.1 | 6.1 | 7.3 | 2.5 | 740.7 |
| IL-12p40 | 0.5 | 0.0 | 0.0 | 1.1 | 0.4 | 0.3 | 3,333.3.00 |
| IL-12p70 | 0.3 | 0.2 | 0.2 | 0.1 | 0.1 | 0.1 | 500.0 |
| IL-13 | 0.9 | 0.0 | 0.0 | 0.0 | 0.7 | 0.7 | 333.3 |
| IL-15 | 0.0 | 0.0 | 0.0 | 0.0 | 0.0 | 2.5 | 4,000.0.00 |
| IL-16 | 0.6 | 0.2 | 0.4 | 0.1 | 0.3 | 0.6 | 1,666.7.00 |
| IL-17 | 2.5 | 1.1 | 1.2 | 2.0 | 2.8 | 1.1 | 1,333.3.00 |
| MCP-1 | 33.0 | 3.6 | 28.2 | 0.0 | 18.4 | 6.9 | 222.2 |
| MCSF | 10.2 | 6.6 | 2.7 | 5.6 | 3.2 | 0.6 | 444.4 |
| MIG | 7.3 | 2.1 | 0.2 | 3.4 | 9.5 | 2.7 | 5,000.0.00 |
| MIP-1α | 10.6 | 0.0 | 0.0 | 11.0 | 16.7 | 4.0 | 10,000.0.00 |
| MIP-1β | 11.6 | 18.0 | 13.5 | 61.3 | 1.9 | 0.9 | 333.3 |
| MIP-1d | 1.4 | 0.7 | 0.0 | 4.6 | 0.0 | 1.0 | 10,000.0.00 |
| PDGF-BB | 72.6 | 33.3 | 45.7 | 82.1 | 91.1 | 3.3 | 2,000.0.00 |
| RANTES | 4.0 | 2.3 | 1.9 | 5.4 | 4.9 | 0.5 | 740.7 |
| TIMP-1 | 1,699.4.00 | 1,513.2.00 | 1,656.0.00 | 1,787.9.00 | 2,111.9.00 | 1.1 | 1,481.5.00 |
| TIMP-2 | 11,523.7.00 | 8,689.8.00 | 8,023.7.00 | 9,953.9.00 | 10,266.3.00 | 3.4 | 13,333.3.00 |
| TNFα | 8.7 | 5.5 | 0.7 | 5.1 | 10.7 | 10.0 | 666.7 |
| TNFβ | 44.5 | 0.0 | 0.0 | 49.0 | 114.5 | 84.6 | 20,000.0.00 |
| TNF RI | 96.6 | 120.9 | 74.0 | 118.4 | 71.1 | 4.9 | 4,444.4.00 |
| TNF RII | 0.0 | 0.0 | 4.4 | 0.0 | 0.0 | 3.4 | 4,444.4.00 |
| 4-1BB | 0.0 | 0.0 | 0.0 | 1.3 | 1.1 | 10.3 | 3,333.3.00 |
| ALCAM | 15.1 | 5.5 | 18.4 | 25.8 | 9.5 | 16.4 | 3,333.3.00 |
| B7-1 | 0.0 | 0.0 | 0.0 | 0.0 | 0.0 | 85.2 | 10,000.0.00 |
| BCMA | 3.2 | 0.0 | 0.0 | 14.6 | 0.0 | 20.2 | 6,666.7.00 |
| CD14 | 4.8 | 9.4 | 0.0 | 19.6 | 7.4 | 10.0 | 3,333.3.00 |
| CD30 | 1,558.5.00 | 1,504.7.00 | 2,660.8.00 | 2,656.8.00 | 1,810.7.00 | 842.5 | 10,000.0.00 |
| CD40L | 0.0 | 2.5 | 0.3 | 5.1 | 0.0 | 3.4 | 3,333.3.00 |
| CEACAM-1 | 1.5 | 2.1 | 2.6 | 18.0 | 0.0 | 26.5 | 10,000.0.00 |
| DR6 | 6.3 | 4.9 | 3.7 | 4.8 | 3.2 | 0.9 | 4,000.0.00 |
| Dtk | 0.0 | 0.0 | 0.0 | 24.2 | 1.3 | 75.6 | 6,666.7.00 |
| Endoglin | 0.0 | 0.0 | 0.0 | 2.8 | 0.0 | 21.7 | 1,333.3.00 |
| ErbB3 | 0.2 | 0.0 | 0.0 | 0.2 | 1.7 | 6.1 | 20,000.0.00 |
| E-Selectin | 0.0 | 0.0 | 11.7 | 17.0 | 0.1 | 44.7 | 13,333.3.00 |
| Fas | 10.8 | 7.7 | 15.0 | 11.7 | 8.8 | 1.8 | 666.7 |
| Flt-3L | 0.3 | 0.0 | 0.7 | 0.0 | 1.9 | 2.9 | 666.7 |
| GITR | 123.8 | 0.0 | 179.4 | 195.5 | 141.9 | 331.4 | 10,000.0.00 |
| HVEM | 24.7 | 0.0 | 0.0 | 144.1 | 0.0 | 340.7 | 40,000.0.00 |
| ICAM-3 | 0.0 | 15.6 | 0.0 | 39.9 | 11.8 | 60.6 | 33,333.3.00 |
| Contactin-2 | 0.0 | 0.0 | 0.0 | 450.4 | 66.5 | 596.1 | 33,333.3.00 |
| IL-1 RI | 80.6 | 0.0 | 134.0 | 441.5 | 64.7 | 359.9 | 4,000.0.00 |
| IL-2 Rg | 2.8 | 2.3 | 1.9 | 2.7 | 2.3 | 1.0 | 370.4 |
| IL-10 Rb | 0.0 | 0.0 | 2.4 | 18.2 | 7.6 | 23.6 | 4,000.0.00 |
| IL-17R | 1.1 | 0.0 | 0.0 | 54.7 | 10.7 | 42.5 | 10,000.0.00 |
| IL-21R | 0.0 | 0.0 | 0.0 | 84.6 | 0.0 | 72.2 | 20,000.0.00 |
| LIMPII | 0.0 | 0.0 | 0.0 | 8.1 | 0.0 | 7.9 | 4,000.0.00 |
| Lipocalin-2 | 0.9 | 0.0 | 0.0 | 0.9 | 0.0 | 0.7 | 1,000.0.00 |
| L-Selectin | 0.0 | 0.0 | 0.0 | 292.0 | 555.3 | 1,126.1.00 | 33,333.3.00 |
| LYVE-1 | 0.0 | 0.5 | 0.4 | 0.7 | 0.0 | 0.4 | 666.7 |
| MICA | 51.5 | 40.6 | 23.4 | 21.6 | 5.0 | 7.9 | 10,000.0.00 |
| MICB | 21.6 | 0.0 | 0.0 | 37.1 | 96.5 | 97.3 | 15,000.0.00 |
| NRG1-b1 | 0.0 | 0.0 | 0.0 | 3.5 | 1.5 | 4.1 | 15,000.0.00 |
| PDGF Rb | 1,182.1.00 | 0.0 | 2,185.5.00 | 2,407.9.00 | 797.1 | 2,257.6.00 | 33,333.3.00 |
| PECAM-1 | 27.6 | 0.0 | 50.0 | 39.1 | 2.7 | 38.4 | 6,666.7.00 |
| RAGE | 11.1 | 1.3 | 7.2 | 14.9 | 6.8 | 8.7 | 3,333.3.00 |
| TIM-1 | 4.6 | 0.0 | 6.8 | 9.8 | 2.6 | 7.4 | 10,000.0.00 |
| TRAIL R3 | 7.1 | 10.1 | 6.8 | 10.4 | 5.4 | 1.7 | 1,666.7.00 |
| Trappin-2 | 0.0 | 0.0 | 0.0 | 60.5 | 0.0 | 6.5 | 3,333.3.00 |
| uPAR | 1,742.9.00 | 921.7 | 968.1 | 688.5 | 1,116.3.00 | 115.9 | 40,000.0.00 |
| VCAM-1 | 794.8 | 267.1 | 365.0 | 2,090.4.00 | 164.8 | 913.9 | 200,000.0.00 |
| XEDAR | 0.0 | 0.0 | 16.6 | 45.7 | 0.0 | 93.8 | 3,333.3.00 |

An exemplary determination of the gene function and processes based upon these results is provided below in Table 2:

TABLE 2

| | | | |
|---|---|---|---|
| Target | Gene ID | Gene name | Gene Info (Function, Process) |
| CCL28 | CCL28 | C-C motif chemokine ligand 28 | chemokine activity; immune response; Innate and adaptive immunity; upregulated by hypoxia |
| ENA-78 | CXCL5 | C-X-C motif chemokine ligand 5 | chemokine activity; immune response, inflammatory response, antimicrobial humoral immune response; leukocyte and neutrophil chemotaxis; |
| Eotaxin-3 | CCL26 | C-C motif chemokine ligand 26 | chemokine activity; chemotactic activity for normal peripheral blood eosinophils and basophils; antimicrobial activity |
| IL-28A | IFNL2 | interferon lambda 2 | cytokine activity; defense response to virus; innate immune response, mucosal immune response |
| IL-29 | IFNL1 | interferon lambda 1 | cytokine activity; defense response to virus; innate immune response |
| I-TAC | CXCL11 | C-X-C motif chemokine ligand 11 | chemokine activity; heparin binding; T cell chemotaxis; immune response |
| Lympho tactin | CXCL1 | X-C motif chemokine ligand 1 | chemokine activity; inflammatory response; lymphocyte, monocyte, neutrophil, NK cell chemotaxis; cell response to IL1, IL4, TNF |
| MCP-2 | CCL8 | C-C motif chemokine ligand 8 | chemokine activity; heparin binding; calcium homeostasis; inflammatory response; eosinophil, lymphocyte, monocyte, neutrophil chemotaxis; cell response to IL1, TNF, IFN-gamma |
| MCP-3 | CCL7 | C-C motif chemokine ligand 7 | chemokine activity; heparin binding; calcium homeostasis; inflammatory response; eosinophil, lymphocyte, monocyte, neutrophil chemotaxis; cell response to IL1, TNF, IFN-gamma |
| MCP-4 | CCL13 | C-C motif chemokine ligand 13 | chemokine activity; calcium homeostasis; inflammatory response; eosinophil, lymphocyte, monocyte, neutrophil chemotaxis; cell response to IL1, TNF, IFN-gamma |
| MIF | MIF | macrophage migration inhibitory factor | cytokine activity; chemoattractant activity; inflammatory response; innate immune response; |
| MIP-3a | CCL20 | C-C motif chemokine ligand 20 | chemokine activity; lymphocyte, monocyte, neutrophil chemotaxis; immune response; inflammatory response; defense response to bacterium |
| NAP-2 | NAP1L4 | nucleosome assembly protein 1 like 4 | RNA, histone, nucleosome binding; nucleosome assembly; cell differentiation; |
| OPN | SPP1 | secreted phosphoprotei n 1 | cytokine activity; ECM binding; biomineral tissue development; embryo implantation; osteoblast differentiation; inflammatory response; |
| PF4 | PF4 | platelet factor 4 | chemokine activity; heparin binding; antimicrobial humoral immune response; defense response to protozoan; immune response; inflammatory response; leukocyte, neutrophil chemotaxis; platelet activation and degranulation; |
| SDF-1a | CXCL12 | C-X-C motif chemokine ligand 12 | chemokine activity; growth factor activity; chemotaxis; blood circulation; axon guidance; defense response; |
| TECK | CCL25 | C-C motif chemokine ligand 25 | chemokine activity; hormone activity; lymphocyte, monocyte, neutrophil chemotaxis; immune response; inflammatory response; cell response to IL1, TNF, IFN-gamma |
| Angioge nin | ANG | angiogenin | DNA, rRNA binding; actin filament polymerization; adherens junctions; angiogenesis; antibacterial, antimicrobial response; innate immune response; placenta development; oocyte maturation; |
| ANG-1 | ANGPT1 | angiopoietin 1 | RTK binding; angiogenesis; cell differentiation; hemopoiesis; in utero embryonic development; |
| DKK-1 | DKK1 | dickkopf WNT signaling pathway inhibitor 1 | growth factor activity; endoderm, mesoderm formation; embryonic limb morphogenesis; forebrain development; expressed in placenta |
| Follistati n | FST | follistatin | cell differentiation; morphogenesis; BMP signaling pathway; multicellular organism development |
| LAP(TG Fb1) | TGFβ1 | transforming growth factor beta 1 | growth factor activity; cytokine activity; cell migration; epithelial to mesenchymal transition; heart, liver development; heart valve, aortic valve morphogenesis; controls cell growth, proliferation, differentiation and apoptosis autoimmunity, oncology, cardiology |
| PAI-1 | SERPINE 1 | serpin family E member 1 | angiogenesis; dentinogenesis; ECM organization; platelet degranulation; |
| VEGF R1 | FLTI | fms related receptor tyrosine kinase 1 | growth factor binding; angiogenesis; blood vessel morphogenesis; cell migration; embryonic morphogenesis; |
| ANGPT L4 | ANGPTL 4 | angiopoietin like 4 | angiogenesis; lipid metabolism; triglyceride homeostasis; response to hypoxia; |
| B2M | B2M | beta-2-microglobulin | T-cell differentiation in thymus; amyloid fibril formation; innate immune response; iron homeostasis; antigen processing and presentation; |
| GROa | CXCL1 | C-X-C motif chemokine ligand 1 | chemokine activity; growth factor activity; immune response; inflammatory response; leukocyte, neutrophil chemotaxis; neutrophil degranulation; |
| IL-21 | IL21 | interleukin 21 | cytokine activity; cell maturation; defense response to virus; immunoglobulin production; |
| MMP-1 | MMP1 | matrix metallopeptid ase 1 | metalloendopeptidase activity; ECM organization; collagen catabolism; leukocyte migration; |
| Nidogen -1 | NID1 | nidogen 1 | ECM structural constituent; basement membrane and ECM organization. |
| PSA-free | KLK3 | kallikrein related peptidase 3 | endopeptidase activity; antibacterial peptide production; |
| Siglec-9 | SIGLEC9 | sialic acid binding Ig like lectin 9 | sialic acid binding; regulation of immune response; cell adhesion; neutrophil degranulation. |
| TIMP-4 | TIMP4 | TIMP metallopeptid ase inhibitor 4 | metallopeptidase inhibitor activity; CNS development; ovulation cycle. |
| FAP | FAP | fibroblast activation protein alpha | peptidase activity; proteolysis; angiogenesis; cell adhesion; endothelial cell migration; |
| Galectin -3 | LGALS3 | galectin 3 | eosinophil, macrophage, monocyteneutrophil chemotaxis; neutrophil degranulation; |
| HGF R | MET | MET proto-oncogene, receptor | hepatocyte growth factor receptor signaling; liver, pancreas, nervous system development; neuron differentiation; establishment of skin barrier; entry of bacterium in host cells. |
| | | tyrosine kinase | |
| Legumai n | LGMN | legumain | endopeptidase activity; antigen processing and presentation; cell response to hepatocyte growth factor stimulus; |
| Thromb omoduli n | THBD | thrombomodu lin | blood coagulation; femal pregnancy; leukocyte migration. |
| Transfer rin | TF | transferrin | iron binding; iron transport and homeostasis; response to bacterium; |
| Albumin | ALB | albumin | fatty acid, copper, drug, protein, zinc, enterobactin binding. |
| Clusteri n | CLU | clusterin | protein binding; antimicrobial humoral response; cell morphogenesis; complement activation; innate immune response. |
| CXCL1 4 | CXCL14 | C-X-C motif chemokine ligand 14 | chemokine activity; antimicrobial humoral immune response; chemotaxis; inhibits angiogenesis, Oncology |
| Cystatin C | CST3 | cystatin C | protease and protein binding; defense response; embryo implantation; tissue remodeling, protease inhibitor/ treatment of infectious diseases |
| Decorin | DCN | decorin | ECM organization, kidney, placenta development; wound healing; collagen cross-linker, oncology |
| Dkk-3 | DKK3 | dickkopf WNT signaling pathway inhibitor 3 | protein binding; Wnt signaling pathway; anatomical structure morphogenesis. Wnt inhibitor, tumor suppressor; oncology |
| Furin | FURIN | paired basic amino acid cleaving enzyme | endopeptidase activity; blastocyst formation, cellular protein metabolic process, ECM organization. |
| LDL R | LDLR | low density lipoprotein receptor | LDL binding; cholesterol metabolism; artery morphogenesis. |
| Pepsino gen I | PGA3 | pepsinogen A3 | aspartic-type endopeptidase activity; digestion, proteolysis |
| RBP4 | RBP4 | retinol binding protein 4 | retinol binding; embryonic organ morphogenesis; embryonic skeletal system development; heart, lung development; glucose homeostasis. |
| TSP-1 | THBS1 | thrombospond in 1 | protein binding; cell adhesion, cell migration, ECM organization; immune response; inflammatory response; inhibits angiogenesis; oncology |
| bIG-H3 | TGFBI | transforming growth factor beta induced | collagen binding; integrin binding; cell adhesion, cell proliferation; ECM organization; chondrocyte differentiation. |
| Cathepsi n B | CTSB | cathepsin B | peptidase activity; proteolysis; collagen catabolic process; epithelial cell. differentiation. |
| EMMP RIN | BSG | basigin (Ok blood group) | protein binding; embryo implantation; |
| FLRG | FSTL3 | follistatin like 3 | protein binding; multicellular organism development (kidney, lung, adrenal gland, male gonad). |
| Follistati n-like 1 | FSTL1 | follistatin like 1 | protein binding; BMP signaling pathway; cell differentiation; multicellular organism development. |
| Galectin -1 | LGALS 1 | galectin 1 | protein binding, RNA binding; T cell costimulation; myoblast differentiation. |
| Periostin | POSTN | periostin | protein binding, ECM constituent; bone regeneration, wound healing, response to hypoxia; ECM organization; supports adhesion and migration of epithelial cells trauma, autoimmunity, cardiology |
| CD99 | CD99 | CD99 molecule (Xg blood group) | protein binding; regulation of immune response; T cell extravasation; |
| Cystatin B | CSTB | cystatin B | RNA binding; protease binding; |
| JAM-A | F11R | F11 receptor | protein binding; virus receptor activity; cell-cell adhesion; inflammatory response; leukocyte migration. |
| Pentraxi n 3 | PTX3 | pentraxin 3 | protein binding; virion binding; ECM organization; innate immune response; inflammatory response; |
| Syndeca n-4 | SDC4 | syndecan 4 | protein binding; cell migration; wound healing; retinoid metabolic process; neural tube closure; ureteric bud development. |
| Glypica n 1 | GPC1 | glypican 1 | FGF binding; laminin binding; cell migration; axon guidance; retinoid metabolic process. |
| IL-7 R alpha | IL7R | interleukin 7 receptor | protein binding; cytokine receptor activity; B cell proliferation; T cell differentiation; immune response; |
| Semaph orin 7A | SEMA7A | semaphorin 7A | protein binding; chemorepellent activity; axon extension and guidance; neural crest development; regulation of inflammatory response. T |
| GP73 | GOLM1 | golgi membrane protein 1 | protein binding; cellular protein metabolic process; |
| Matrilin -3 | MATN3 | matrilin 3 | protein binding; ECM constituent; cartilage development; ECM organization; skeletal system development; |
| Nidogen -2 | NID2 | nidogen 2 | protein binding; ECM constituent; basement membrane organization; ECM organization; cell-matrix adhesion; |
| Progran ulin | GRN | granulin precursor | |
| TLR3 | TLR3 | toll like receptor 3 | dsRNA binding; protein binding; defense response to bacterium and virus; detection of virus; innate immune response; |
| TPP1 | TPP1 | tripeptidyl peptidase 1 | endopeptidase activity; protein binding; CNS development; proteolysis; epithelial cell differentiation. |
| CRIM1 | CRIM1 | cysteine rich transmembran e BMP regulator 1 | insulin-like growth factor receptor signaling pathway; nervous system development. |
| Lumican | LUM | lumican | collagen binding; ECM structural constituent; protein binding; cartilage development; ECM organization |
| htPAPP-A | PAPPA | pappalysin 1 | metallopeptidase activity; female pregnancy; response to FSH; response to glucocorticoid; proteolysis. |
| OSCAR | OSCAR | osteoclast associated Ig-like receptor | collagen-receptor activity; collage-activated signaling pathway; neutrophil degranulation; osteoclast differentiation; regulation of immune response. |
| ADAM TSL-1 | ADAMTS L1 | ADAMTS like 1 | metallopeptidase activity; ECM organization; proteolysis; |
| Cyr61 | CCN1 | cellular communicatio n network factor 1 | protein binding; ECM structural component; chemotaxis; chondroblast, osteoblast differentiation; ECM organization; atrial septum morphogenesis. |
| DAPP1 | DAPP1 | dual adaptor of phosphotyrosi ne and 3-phosphoinosit ides 1 | phospholipid binding; protein binding; protein dephosphorylation; signal transduction |
| NUP85 | NUP85 | nucleoporin 85 | protein binding; structural component of the nuclear pore; intracellular transport of virus; macrophage chemotaxis; nephron development; viral process; viral transcription. |
| UNC5H 3 | UNC5C | unc-5 netrin receptor C | netrin receptor activity; protein binding; axon guidance; brain development; regulation of cell migration. |
| Visfatin | NAMPT | nicotinamide phosphoribos yltransferase | cytokine activity; protein binding; NAD biosynthesis; female pregnancy; signal transduction; cellular response to oxygen-glucose deprivation. |
| Bcl-10 | BCL10 | BCL10 immune signaling adaptor | NF-kappaB binding; protein binding; B cell, T cell apoptosis; adaptive immune response; innate immune response; cellular defense response; cell death; |
| CD42b | GP1BA | glycoprotein Ib platelet subunit alpha | protein binding; thrombin-activated receptor activity; blood coagulation; platelet activation; platelet aggregation |
| CES1 | CES1 | carboxylestera se 1 | carboxylic ester hydrolase activity; sterol esterase activity; triglyceride lipase activity; cholesterol metabolic process; epithelial cell differentiation; response to toxic substances; xenobiotic metabolic process |
| cIAP-1 | BIRC2 | baculoviral IAP repeat containing 2 | NIK/NF-kappaβ signaling; apoptotic process; placenta development |
| GDF-15 | GDF15 | growth differentiation factor 15 | cytokine activity; growth factor activity; BMP signaling pathway; TGFβ receptor signaling pathway |
| HGF | HGF | hepatocyte growth factor | chemoattractant activity; growth factor activity; animal organ regeneration; cell morphogenesis; cell chemotaxis; epithelial cell proliferation; epithelial to mesenchymal transition |
| IGFBP-2 | IGFBP2 | insulin like growth factor binding protein 2 | protein binding; regulation of growth; cellular response to hormone stimulus; female pregnancy; bind and sequester the insulin-like growth factors and insulin; diabetes, autoimmunity, oncology |
| IGFBP-3 | IGFBP3 | insulin like growth factor binding protein 3 | protein binding; osteoblast differentiation; regulation of cell growth; regulation of apoptotic process; bind and sequester the insulin-like growth factors and insulin; diabetes, autoimmunity, oncology |
| IGFBP-4 | IGFBP4 | insulin like growth factor binding protein 4 | protein binding; inflammatory response; regulation of cell growth; bind and sequester the insulin-like growth factors and insulin; diabetes, autoimmunity, oncology |
| IGFBP-6 | IGFBP6 | insulin like growth factor binding protein 6 | protein binding; cell migration; negative regulation of cell proliferation; regulation of insulin-like growth factor receptor signaling pathway. bind and sequester the insulin-like growth factors and insulin; diabetes, autoimmunity, oncology |
| Insulin | INS | insulin | hormone activity; protein binding; glucose and fatty acid homeostasis |
| IL-6 | IL6 | interleukin 6 | cytokine activity; growth factor activity; cellular response to virus; defense response to virus and bacterium; endocrine pancreas development; glucagon secretion; glucose homeostasis; hepatic immune response; inflammatory response |
| MCSF | CSF1 | colony stimulating factor 1 | cytokine activity; growth factor activity; inflammatory response; innate immune response; macrophage differentiation; osteoclast differentiation |
| MIP-1b | CCL4 | C-C motif chemokine ligand 4 | chemokine activity; cytokine activity; cell adhesion; cellular response to IFN-gamma, IL1, TNF; eosinophil, lymphocyte, monocyte, neutrophil chemotaxis; response to virus; response to toxic substance; immune response |
| PDGF-BB | PDGFB | platelet derived growth factor subunit B | chemoattractant activity; growth factor activity; cell chemotaxis; ECM organization; embryonic placenta development; heart development; monocyte chemotaxis |
| RANTE S | CCL5 | C-C motif chemokine ligand 5 | chemokine activity; chemoattractant activity; cellular response to IL1, IFN-gamma, TNF; inflammatory response; dendritic cell, eosinophil chemotaxis |
| TIMP-1 | TIMP1 | TIMP metallopeptid ase inhibitor 1 | cytokine activity; growth factor activity; peptidase inhibitor activity; response to cytokine, hormone; cartilage development; connective tissue replacement involved in inflammatory response wound healing; ECM disassembly; suppress proliferation of endothelial cells and inhibits protease activity; oncology, neurodegeneration, autoimmunity |
| TIMP-2 | TIMP2 | TIMP metallopeptid ase inhibitor 2 | peptidase inhibitor activity; CNS development; ECM disassembly; response to cytokine, drug, hormone; suppress proliferation of endothelial cells and inhibits protease activity; oncology, neurodegeneration, autoimmunity |
| TNF RI | TNFRSF1 A | TNF receptor superfamily member 1A | TNF binding; I-kappaβ/NF-kappaβ signaling; aortic valve development; defense response to bacterium; inflammatory response |
| DR6 | TNFRSF2 1 | TNFRSF21 T NF receptor superfamily member 21 | protein binding; adaptive immune response; apoptotic process |
| Fas | FAS | Fas cell surface death receptor | protein binding; apoptotic process; immune response |
| TRAIL R3 | TNFRSF1 OC | TNF receptor superfamily member 10c | protein binding; regulation of apoptotic process |
| uPAR | PLAUR | plasminogen activator, urokinase receptor | protein binding; blood coagulation; fibrinolysis; chemotaxis |
| Tumor necrosis factor receptor superfa mily-8 (CD30) | | | Positive regulator of apoptosis and protects against T-cell mediated autoimmunity Oncology |

Hypoxia was found to induce expression of a multitude of proteins, which proteins can be utilized in a secretome as described above.

The secretome is a promising cell-free alternative to cell-based therapies. The secretome is dynamic in its therapeutic effects and can be engineered and customized to intended applications in oncology, regenerative medicine, and cosmeceuticals.

While some embodiments have been shown and described herein, such embodiments are provided by way of example only. Numerous variations, changes, and substitutions can occur without departing from the inventions. It should be understood that various alternatives to the embodiments of the inventions described herein can be employed in practicing the inventions.

What is claimed is:

1. A composition comprising 1) about 0.1% or more w/w of secretome and 2) a pharmaceutically or cosmetically acceptable excipient; wherein the secretome comprises MCP-1 and CXCL2 (GRO), and wherein the composition is free from a cell.

2. The composition of claim 1, wherein the secretome further comprises one or more of IL-6, IL-8, and VEGF proteins.

3. The composition of claim 2, wherein the secretome comprises two of CXCL2 (GRO), IL-6, IL-8, and VEGF proteins.

4. The composition of claim 2, wherein the secretome comprises three of CXCL2 (GRO), IL-6, IL-8, and VEGF proteins.

5. The composition of claim 2, wherein the secretome comprises all of CXCL2 (GRO), IL-6, IL-8, and VEGF proteins.

6. The composition of claim 1, wherein the secretome is present in the composition in an amount of at least 0.6%, 1%, 1.25%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% by weight.

7. The composition of claim 1, wherein the secretome is present in the composition in an amount of from about 2.5% to about 10% by weight.

8-10. (canceled)

11. The composition of claim 1, wherein the composition comprises a liposome that comprises a phospholipid and secretome, and a pharmaceutically or cosmetically acceptable excipient, and wherein the composition is free from a cell.

12. The composition of claim 11, wherein the secretome is encapsulated in the liposome.

13. The composition of claim 11, wherein the liposome is in a form of nanoparticles.

14. The composition of claim 13, wherein the nanoparticles have an average particle size of from about 10 to about 400 nanometers in diameter.

15. The composition of claim 13, wherein the nanoparticles have an average particle size of from about 50 to about 300 nanometers in diameter.

16. The composition of claim 13, wherein the nanoparticles have an average particle size of from about 100 to about 200 nanometers in diameter.

17. (canceled)

18. The composition of claim 11, wherein the secretome comprises micro-vesicles, exosomes, or a combination thereof.

19-41. (canceled)

42. The composition of claim 1, wherein the composition further comprises one or more proteins of IP-10, Eotaxin, Flt-3L, GM-CSF, MIP-1α, MIP-1β, IL-1a, IL-1RA, IL-4, IL-7, IL-10, IL-12P40, IL-13, IL-15, IL-17A, CCL5 (RANTES), MDC, MCP-3, IL-12P70, IFN-alpha, IFN-receptor, PDGF-AB/BB, or EGF.

43. The composition of claim 1, wherein the composition comprises a hydrophilic active agent.

44. The composition of claim 1, wherein the composition comprises a vitamin.

45. The composition of claim 1, wherein the composition comprises a hydrophobic active agent.

46. The composition of claim 1, wherein the composition comprises a fatty acid molecule.

47. The composition of claim 1, wherein the composition comprises linoleic acid.

48. The composition of claim 1, wherein the composition comprises collagen.

49. The composition of claim 1, wherein the composition comprises hyaluronic acid.

50. The composition of claim 1, wherein the composition is free from a serum, antibiotic, or a combination thereof.

51. The composition of claim 1, wherein the composition is free from a steroid, cholesterol, choline chloride, hypoxanthine-sodium salt, thymidine, putrescine dihydrochloride, ferric nitrate, L-glutamine, or any combination thereof.

52. The composition of claim 1, wherein the composition is free from a color additive.

53. The composition of claim 1, wherein the composition is a dosage form of a lotion, cream, liquid, gel, emulsion, suspension, paste, stick, aerosol, foam, patch, powder, ointment, bead, mask, pad, sheet, wound dressing, bandage, or any combination thereof.

54. The composition of claim 1, wherein the pharmaceutically or cosmetically acceptable excipient comprises water, glycerol, a seed oil, a fruit oil, a flower extract, a mineral oil, a synthetic oil, a saccharide, a silicate, a calcium salt, a magnesium salt, sodium chloride, sodium hydroxide, potassium chloride, lactose, lactic acid, a starch, a sugar alcohol, a cellulose, an activated charcoal, an amino acid, a paraffin, honey, a wax, beeswax, an agar, calcium carbonate, a citric acid, tartaric acid, a steric acid, xanthan gum, benzoic acid or salt thereof, a polyethylene glycol, a silicon, or any combination thereof.

55. A method of treating a skin disease or condition in a subject in need thereof, comprising administering a composition of claim 1 to the skin of the subject, whereby the skin disease or condition is treated.

56-507. (canceled)
